(19) United States
(12) Patent Application Publication
(10) Pub. No.: US 2011/0177156 A1
(43) Pub. Date: Jul. 21, 2011

(54) Sterol-Modified Amphiphilic Lipids

(76) Inventors: Francis C. Szoka, JR., San Francisco, CA (US); Zhaohua Huang, San Francisco, CA (US)

(21) Appl. No.: 12/742,594

(22) PCT Filed: Nov. 10, 2008
(86) PCT No.: PCT/US08/83035

§ 371 Date: Dec. 30, 2010

Related U.S. Application Data
(60) Provisional application No. 60/988,038, filed on Nov. 14, 2007.

(51) Int. Cl. A61K 9/127; C07J 51/00; A61K 31/203; A61K 33/24; A61K 31/7115; G01N 33/92; A61P 9/10

(52) U.S. Cl. 424/450; 552/506; 514/559; 424/649; 514/44 A; 436/71

(57) ABSTRACT

Disclosed are sterol-modified amphiphilic lipid compounds having two or more hydrophobic tails of which at least one is a sterol. Also disclosed are the processes for the synthesis of these compounds, compositions comprising such compounds, and the use of such compounds in delivery of an agent of interest, e.g., therapeutics, imaging agents, contrast materials for ultrasound applications, vaccines, biosensors, nutritional supplements and skin care products.

### CROSS-REFERENCE TO RELATED APPLICATION

This application claims priority benefit of U.S. provisional application Ser. No. 60/988,038, filed Nov. 14, 2007, which application is incorporated herein by reference in its entirety.

### GOVERNMENT RIGHTS

This invention was made with government support under federal grant no. R01-GM061851 awarded by The National Institute of Health. The United States Government has certain rights in this invention.

### TECHNICAL FIELD

The present invention relates to amphiphilic lipid compounds, as well as compositions and methods of use.

### BACKGROUND

Eukaryotic membranes have a bilayer structure and are principally composed of phospholipids, sphingolipids and cholesterol. Of these components, cholesterol or a cholesterol-like sterol is the most abundant single chemical species in eukaryotic membranes. Therefore there is considerable interest in understanding the properties and function of cholesterol in biological membranes and cholesterol's role in certain diseases.

Artificial vesicles can be prepared from phospholipids, sphingolipids and other amphipathic synthetic lipids. These artificial vesicles, known as liposomes, have been used in many applications such as models of bilayer membranes and as drug delivery vehicles.

Free cholesterol has been widely used as a component in liposome compositions. When present in the lipid mixture at greater than about 30 mole percent, free cholesterol facilitates liposome bilayer stabilization. Such free cholesterol-containing liposomes are used in drug delivery applications, for imaging agents and in biophysical studies. The properties of mixtures of free cholesterol and synthetic phospholipids are well characterized. For a two component mixture containing free cholesterol and phospholipids, the mole percent of cholesterol plus mole percent of phospholipids equals 100 mole percent. The maximum mole percent of cholesterol that can be included in such lipid mixtures is found to be about 50 mole percent. The inclusion of a high mole percent of free cholesterol (greater than 30 mole %) in the composition containing a synthetic phospholipid will abolish detectable heat of transition of diacyl phospholipids and eliminate the detectable phase transition that is observed in liposomes formed from the synthetic phospholipid. Liposomes containing a high mole percentage of free cholesterol are generally more stable and less leaky than those without free cholesterol, and are widely used in the formulation of chemotherapeutic drugs.

However, when liposomes composed of free cholesterol and phospholipids are placed in biological fluids that contain other biological lipids and serum, free cholesterol rapidly transfers out of the liposome into the biological lipids. This loss of free cholesterol from the liposome usually results in the decrease of stability of lipid bilayer and the subsequent loss of encapsulated contents from the liposome. The half-life for transfer of free cholesterol out of a liposome to an excess of non-cholesterol containing lipids is about 2 hours. Additionally, the presence of serum will significantly increase the leakage of the liposome due to the transfer of the free cholesterol from the liposome to the proteins due to absorption of free cholesterol by serum lipoproteins. This leakage problem associated with the rapid transfer of free cholesterol from the liposome to biological membranes could not be satisfactorily solved by conventional liposome formulations in which free cholesterol is physically mixed with other amphipathic lipid components.

It would, therefore, be desirable to develop lipids that could be used to form stable liposomes both in vitro and in vivo. The desired molecules should not only be able to incorporate a sufficient amount of sterols in the formulation to provide a stabilizing effect, but also to keep the sterols in the formulations when exposed to biological fluids.

The use of water soluble sterol derivatives has been described. A variety of hydrophilic groups have been attached to sterols to make water soluble sterol derivatives such as sterol-hemisuccinate, sterol-phosphocholine, sterol-polyethylene glycol, and sterol-sulfate. Since these molecules have only one sterol as the hydrophobic moiety and a relatively large hydrophilic head group, they tend to form micelles by themselves and transfer rapidly from the liposome bilayer to the biological lipids. Therefore, such water soluble sterols are not suitable components for stable liposome formulations.

Hydrophobic sterols are another known category of sterol derivatives in which a fatty acid ester or alkyl ether is attached to the sterol. Although these molecules help to retain the sterol in the liposome bilayer due to the aliphatic chain, only a low mole percent (less than 15%) can be incorporated in the formulation. When the percentage of the free cholesterol ester is greater than about 15 mole percent, the cholesterol ester phase separates into a separate phase that is not incorporated into a bilayer. Thus, such hydrophobic sterols can not be introduced at a sufficient mole percent of sterol into the lipid composition to eliminate the phase transition and stabilize the liposome bilayer.

The present invention provides compounds that can solve these problems, and provides for liposomes having desired physical properties.

### LITERATURE

Sterols, lipids and delivery formulations in general are reviewed in: Fahy et al., J. Lipid Research (2005) 46:839-861; Felgner, 1990, Advanced Drug Delivery Reviews, 5:162-187; and Felgner 1993, J. Liposome Res., 3:3-16.

Lipid compounds and compositions containing cholesterol are disclosed in the following literature, for example: Brockerhoff et al., Biochim. Biophys. Acta 1982, 691:227-232; Demel et al, Biochim. Biophys. Acta 1984, 771:142-150; Patel et al, Biochim. Biophys. Acta 1984, 797:20-26; Lai et al, Biochemistry 1985, 24:1646-1653; Epand et al, Chem. Phys. Lipids 1990, 55:49-53; Gotoh et all., Chem. Biodivers. 2006, 3:198-209; Torchilin, V. P., Nat. Rev. Drug Discov. 2005, 4:145-60; Phillips et al., Biochim. Biophys. Acta 1987, 906:223-76; Hamilton, J. A., Curr. Opin. Lipidol. 2003, 14:263-271; Kan et al., Biochemistry 1992, 31:1866-74; Urata et al, Eur. J. Lipid Sci. Tech. 2001, 103:29-39; Salunke et al., Curr. Med. Chem. 2006, 13:813-47; Guo et al., Acc. Chem. Res. 2003, 36:335-341; Bhattacharya et al., Curr. Opin. Chem. Biol. 2005, 9:647-55; and Huang et al., In Liposome technology 3rd E, Gregoriadis, G., Ed., Informa Healthcare: New York, 2007, Vol. 1, p 165-196. For instance, Bhattacharya et al. (Curr. Opin. Chem. Biol. 2005, 9:647-55) review lipid design and their utilization in various biochemical, physical and chemical applications.

Lipid compounds and compositions for delivery of nucleic acids are disclosed in the following patent documents, for example: U.S. Pat. No. 4,493,832; U.S. Pat. No. 4,897,355; U.S. Pat. No. 5,651,981; U.S. Pat. No. 5,661,018; U.S. Pat. No. 5,686,620; U.S. Pat. No. 5,688,958; U.S. Pat. No. 5,780,053; U.S. Pat. No. 5,855,910; U.S. Pat. No. 5,891,714; U.S. Pat. No. 6,627,218; Lewis et al., U.S. Patent Application Publication No. 20030125281; MacLachlan PCT Publication No. WO/2003/077829; MacLachlan, PCT Publication No. WO 05/007196; Vargeese et al., PCT Publication No. WO2005007854; McSwiggen et al. PCT Publication Nos. WO 05/019453, WO 03/70918, WO 03/74654 and US patent Application Publication No. 20050020525 and 20050032733; and Chen et al., PCT Publication No. WO/2007/086883. For instance, Chen et al. (PCT Publication No. WO/2007/086883) disclose cationic lipids, microparticles, nanoparticles and transfection agents that transfect or deliver short interfering nucleic acid (siNA).

### SUMMARY

The present invention generally relates to sterol-modified amphiphilic lipid compounds. Also provided are methods for the synthesis of these compounds, compositions comprising such compounds, and the use of such compounds in delivery of an agent of interest, e.g., therapeutics, vaccines, imaging agents, contrast materials for ultrasound applications, biosensors, nutritional supplements, skin care products and cosmetics.

The sterol-modified amphiphilic lipid compounds of the invention comprise a hydrophilic head group and two or more hydrophobic tail groups, wherein at least one of the hydrophobic tail groups comprises a sterol.

The compounds and compositions of the invention can be adapted for a variety of pharmaceutical, cosmetic, and medical applications, as well as an array of industrial and commercial applications in which lipid systems find use. For example, the compounds of the present disclosure can be used to, for example, stabilize bilayers, monolayers, cubic phases, hexagonal phases, oil and water emulsions (oil-in-water or water-in-oil emulsions), gels, foams, lotions, and creams.

Accordingly, in one aspect the disclosure provides a compound comprising a sterol-modified amphiphilic lipid having a hydrophilic head group and two or more hydrophobic tail groups, wherein at least one of the hydrophobic tail groups comprises a sterol. In one embodiment, the sterol is selected from the group consisting of zoosterols and phytosterols. In related embodiments, the sterol is selected from the group consisting of cholesterol, steroid hormones, campesterol, sitosterol, ergosterol, and stigmasterol.

In still further embodiments, the hydrophilic head group is selected from the group consisting of charged, polar and a combination charged and polar head groups. In related embodiments, the hydrophilic head group is selected from the group consisting of phosphate, phosphocholine, phosphoglycerol, phosphoethanolamine, phosphoserine, phosphoinositol, ethylphosphosphorylcholine, polyethyleneglycol, polyglycerol, melamine, glucosamine, trimethylamine, polyamine, hydroxyl (OH), carboxylate (COO⁻), sulfate (SO₄⁻), sulfonate (SO₃⁻) and carbohydrate.

In further embodiments, at least one of the hydrophobic tail groups comprises a non-sterol. In related embodiments, the non-sterol is an aliphatic hydrocarbon that is saturated or unsaturated, linear or branched, substituted or unsubstituted. In further related embodiments, the non-sterol moiety is a substituted aliphatic hydrocarbon chain that is based on a saturated aliphatic hydrocarbon chain, such as a chain saturated with alkyl groups. In further related embodiments, one, two, three, four or more carbon atoms (generally no more than about 10 carbon atoms) of the alkylene groups are substituted by a heteroatom selected from oxygen, silicon, sulphur or nitrogen atoms, and/or one, two, three, four or more hydrogen atoms (generally no more than the total number of hydrogen atoms) in the alkylene groups is substituted with fluoride.

In further embodiments, the sterol-modified amphiphilic lipid is selected from the group consisting of a monosterol-modified amphiphilic lipid, and a disterol-modified amphiphilic lipid. In related embodiments, the disterol-modified amphiphilic lipid comprises sterol hydrophobic tails that are the same. In further embodiments, the sterol-modified amphiphilic lipid is selected from the group consisting of glycerophospholipids, sphingophospholipids, carnitine lipids, and amino acid lipids.

In still further embodiments, the hydrophilic head group and the hydrophobic tail groups are linked through 1,2-dihydroxy,3-amino propane.

In one embodiment, one of the hydrophobic tail groups is a prodrug, such as retinoic acid.

In further aspects the disclosure provides, a composition comprising a sterol-modified amphiphilic lipid having a hydrophilic head group and two or more hydrophobic tail groups, and wherein at least one of the hydrophobic tail groups comprises a sterol. In related embodiments, the sterol-modified amphiphilic lipid is selected from the group consisting of a monosterol-modified amphiphilic lipid, and a disterol-modified amphiphilic lipid. In further embodiments, the sterol-modified amphiphilic lipid is selected from the group consisting of cholesterol, steroid hormones, campesterol, sitosterol, ergosterol, and stigmasterol.

In related embodiments, the composition is an emulsion. In further related embodiments, the composition is a liposome, which optionally comprises a payload. In embodiments where the liposome comprises a payload, the payload comprises at least one of a therapeutic agent, a cosmetic agent, and a detectable label. In further embodiments, the liposome comprises a non-sterol amphiphilic lipid. In related embodiments, the liposome comprises one or more excipients, and may be provided as a pharmaceutical preparation or a cosmetic preparation.

In further embodiments, the sterol-modified amphiphilic lipid comprises a therapeutic agent, which sterol-modified amphiphilic lipid may be provided in a liposome.

In still further embodiments, the non-sterol amphiphilic lipid is selected from the group consisting of an aliphatic hydrocarbon that is saturated or unsaturated, linear or branched, substituted or unsubstituted. In further embodiments, the sterol-modified amphiphilic lipid and the non-sterol amphiphilic lipid comprise hydrophobic tail groups that are approximately the same lengths. In some embodiments, the sterol-modified amphiphilic lipid of the composition is a monosterol-modified amphiphilic lipid.

In other aspects the disclosure provides methods for synthesis of a sterol-modified amphiphilic lipid, the method comprising coupling at least one sterol tail group through a branching core to a hydrophilic head group so as to generate a sterol-modified amphiphilic lipid having a hydrophilic head group linked to two or more hydrophobic tail groups, wherein at least one of the hydrophobic tail groups comprises the sterol tail group.

In further aspects, the disclosure provides methods for the production of a composition comprising a sterol-modified amphiphilic lipid, the method comprising admixing a sterol-modified amphiphilic lipid with at least one of a non-sterol amphiphilic lipid, a therapeutic agent, a cosmetic agent, a detectable label, a buffer, a solvent, and an excipient. In related embodiments, the method further comprises purifying the composition.

In still other aspects the disclosure provides methods of administering a composition comprising a sterol-modified amphiphilic lipid to an animal, the method comprising contacting the animal with a sterol-modified lipid composition of the present disclosure.

In further aspects the disclosure provides methods of administering a composition comprising a sterol-modified amphiphilic lipid to a cell, the method comprising contacting the cell with a composition comprising a sterol-modified lipid composition of the present disclosure.

In yet other aspects the disclosure provides methods of detecting the presence or absence of an analyte in fluid comprising contacting the fluid with a composition comprising a sterol-modified lipid composition of the present disclosure and detecting at least one change in a detectable property of the lipid composition or the fluid. In related embodiments, the fluid is a biological fluid. In further related embodiments, detecting is by evaluation of a property of the lipid composition.

Other aspects and embodiments of the invention will be readily apparent upon reading the present disclosure.

### BRIEF DESCRIPTION OF THE DRAWINGS

The invention may be best understood from the following detailed description when read in conjunction with the accompanying drawings. Included in the drawings are the following figures:

FIG. 1 is a differential scanning calorimetery (DSC) thermogram of mixtures of SCh_{c}PC and DSPC.

FIG. 2 is a graph illustrating the transition temperature and enthalpy of sterol-modified amphiphilic lipid (“SML”)/diacyl lipid mixtures with various percentage of cholesterol. In the results illustrated in FIG. 2, the SMLs are mixed with diacyl lipids of the same chain length.

FIG. 3 is a graph illustrating the results of analysis of osmotic stress-induced leakage. The release of calcein from the liposomes was measured under different osmotic pressures. The fraction of calcein remained in the liposome was calculated and plotted versus the osmotic gradient. The error of data is within 0.5%.

FIG. 4 is a graph illustrating the results of analysis of leakage of liposome compositions in 30 volume % fetal bovine serum in phosphate buffer saline. The release of calcein from liposome in 30 volume % fetal bovine serum at 37° C. was monitored by measuring the change in the fluorescence intensity from the starting fluorescent intensity at different incubation times. The fraction of calcein remained in the liposome was calculated and plotted versus the time of incubation. There was 40% cholesterol in the control formulations (DSPC/Chol and DMPC/Chol).

FIG. 5 is a graph illustrating the results of analysis of relative rates of cholesterol exchange at 37° C.

FIG. 6 is a graph illustrating the cytotoxicity of selected sterol-modified amphiphilic lipids on C26 colon carcinoma cells

FIG. 7 is a graph showing hydrolysis of all-trans-retinoic acid from sterol-modified amphiphilic lipid by phospholipase A2.

FIG. 8 is a graph showing tumor growth curves for BALB/c mice bearing s.c. C-26 tumors treated with different formulation of doxorubicin encapsulated SPL liposomes, 15 mg/kg i.v., where F1 is SeChcPC/DSPE-PEG2000/α-Tocopherol, 94.8/5.0/0.2; F2 is SeChcPC/DSPE-PEG5000/α-Tocopherol, 94.8/5.0/0.2; F3 is DChcPC/DSPC/DSPE-PEG2000/α-Tocopherol, 10.6/84.2/5.0/0.2; F4 is PChcPC/DSPE-PEG2000/α-Tocopherol, 94.8/5.0/0.2; F5 is DCHEMSPC/DSPC/DSPE-PEG2000/α-Tocopherol, 33/61.8/5.0/0.2; and F6 is DCHEMSPC/DSPE-PEG2000/α-Tocopherol, 94.8/5.0/0.2.

FIG. 9 is a graph showing survival curves for BALB/c mice bearing s.c. C-26 tumors treated with different formulation of doxorubicin encapsulated SPL liposomes, 15 mg/kg i.v, where F1 is SeChcPC/DSPE-PEG2000/α-Tocopherol, 94.8/5.0/0.2; F2 is SeChcPC/DSPE-PEG5000/α-Tocopherol, 94.8/5.0/0.2; F3 is DChcPC/DSPC/DSPE-PEG2000/α-Tocopherol, 10.6/84.2/5.0/0.2; F4 is PChcPC/DSPE-PEG2000/α-Tocopherol, 94.8/5.0/0.2; F5 is DCHEMSPC/DSPC/DSPE-PEG2000/α-Tocopherol, 33/61.8/5.0/0.2; and F6 is DCHEMSPC/DSPE-PEG2000/α-Tocopherol, 94.8/5.0/0.2.

FIG. 10 is a differential scanning calorimetery (DSC) thermogram of DChcPC/DPPC mixtures.

FIG. 11 is a schematic illustrating sequential assembly of nanolipoparticle using reduction-sensitive SML lipid. First, DNA is encapsulated into the nano-sized cationic liposome by the dialysis method. Then the particle surface is modified by disulfide exchange with the reducing agent HSR′. Finally the targeting ligand is incorporated onto the surface of the particle either by the micelle transfer method or through disulfide exchange. SML-SSR: cationic sterol-modified lipid having disulfide bond (SS) in the head group (R); SML-SSR': head group exchanged sterol-modified amphiphilic lipid.

FIG. 12 is a graph illustrating the results of analysis of leakage of liposome compositions in 30 volume % fetal bovine serum in HEPES buffer saline. The release of 5-carboxyfluorescein (CF) from liposome in 30 volume % fetal bovine serum at 37° C. was monitored by measuring the change in the fluorescent intensity from the starting fluorescent intensity at different incubation times. The percentage of CF released from the liposome at day 28 was calculated and plotted versus the alkyl chain length in the liposome formulation.

FIG. 13 is a graph illustrating the profiles of CF release from liposomes comprising of diacyl/SML6b in 30 volume % fetal bovine serum in HEPES buffer saline at 37° C. Labels indicate the chain length of the diacyl lipids used in the liposomes.

### DETAILED DESCRIPTION

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a sterol-modified amphiphilic lipid” includes a plurality of such sterol-modified amphiphilic lipids and reference to “the liposome” includes reference to one or more liposomes, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

“Amphipathic lipids” refers to molecules that are mostly lipid-like (hydrophobic) in structure, but at one end have a region that is polar, charged, or a combination of polar and charged (hydrophilic). The hydrophilic region is referred to as the head group, and the lipid portion is known as the tail group(s). Examples of amphipathic lipids include phospholipids, glycolipids, and sphingolipids.

“Analyte” refers to a substance or chemical constituent that is determined in an analytical or qualitative detection procedure, such as a titration, immunoassay, chromatography, spectrophotometry, thermography and the like. An analyte itself typically cannot be measured, but a measurable property of the analyte can. For instance, typical properties measured or detected are concentration, optical absorbance, molecular weight, melting temperature, binding properties, biological activity, and so forth.

“Free sterol” refers to a sterol that is not covalently bound to another compound. Thus, “free cholesterol” refers to cholesterol that is not covalently bound to another compound. For example, addition of free cholesterol to lipids has been used as a conventional technique to provide for enhanced liposome stability. Thus “free cholesterol” particularly refers to cholesterol that is not covalently bound as a moiety in a sterol-modified amphiphilic lipid compound.

“Pharmaceutical agent” refers to an agent that finds use in the testing, development or application as a pharmaceutical, including nutraceuticals.

“Multivesicular liposomes” (MVL) refers to liposomes containing multiple non-concentric chambers within each liposome particle, resembling a “foam-like” matrix.

“Multilamellar liposomes” (also known as multilamellar vesicles or “MLV”) refers to liposomes that contain multiple concentric chambers composed of bilayers within each liposome particle, resembling the “layers of an onion”.

“Monosterol-modified amphiphilic lipid” or “m-SML” refers to SMLs having only one sterol in the structure

“Disterol-modified amphiphilic lipid” or “di-SML” refers to SML having two sterols sterols in the structure.

“Tristerol-modified amphiphilic lipid” or “tri-SML” refers to SML having three sterols in the structure.

“Tetrasterol-modified amphiphilic lipid” or “tetra-SML” refers to SML having four sterols in the structure.

“Sterol” or steroid alcohols refer to the subgroup of steroids having a free hydroxyl or a derivative thereof, such as exemplified by and encompassed in the class cholesterol and derivatives thereof, as well as the classes phytosterols and derivatives thereof, and fungal sterols and derivatives thereof. Sterols can be natural or synthetic.

“Sterol-modified amphiphilic lipid” as used herein refers generally to amphiphilic lipid compounds having a hydrophilic head group, and two or more hydrophobic tails of which at least one is sterol. “Sterol-modified amphiphilic phospholipids” or “SPL” refers to a sterol-modified amphiphilic lipid comprising a phosphate-containing moiety, such as phosphocholine or phosphoglycerol.

“Therapeutic agent” refers to an agent that finds use in the testing, development or application as a therapeutic, including pharmaceutical agents.

“Imaging agent” refers to an agent that finds use in locating the position of a lipid particle in an animal, including: optical agents, ultrasound contrast agents, high mass X-ray contrast agents, radioactive imaging agents or nuclear magnetic imaging agents.

“Cosmetic agent” refers to an agent that finds use in the testing, development or application as a cosmetic.

The terms “therapeutically acceptable”, “pharmaceutically acceptable” and “cosmetically acceptable” refer to a material that is not biologically or otherwise undesirable, i.e., the material is of an acceptable quality and composition that may be administered to an individual along with the selected active ingredient without causing any undesirable biological effects or interacting in a deleterious manner with any of the other components of the composition in which it is contained.

The term “emulsion” refers to a mixture of two immiscible (unblendable) substances.

The term “bilayer” refers to a “sandwich-like” structure composed of amphiphilic lipid molecules (often phospholipids) that are arranged as two molecular layers with the hydrophobic tails on the inside and the polar head groups on the outside surfaces.

The term “monolayer” refers to a structure defined by a molecular layer of amphipathic molecules with the head groups enriched and substantially aligned on one side and hydrophobic groups enriched and substantially on the opposite side.

The term “excipient” as used herein refers to any suitable substance which provides an acceptable vehicle for a given end use, such as in the application or administration of a compound(s) of interest to a subject. Examples of excipients include substances referred to as diluents, additives, adjuvants, and carriers. For example, a “pharmaceutically acceptable excipient,” “pharmaceutically acceptable diluent,” “pharmaceutically acceptable carrier,” and “pharmaceutically acceptable adjuvant” includes excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use, and may include both one and more than one such excipient, diluent, carrier, and adjuvant.

The term “physiological conditions” is meant to encompass those conditions compatible with living cells, e.g., predominantly aqueous conditions of a temperature, pH, salinity, etc. that are compatible with living cells.

The terms “therapeutic composition”, “pharmaceutical composition”, “cosmetic compositions”, “therapeutic preparation”, “pharmaceutical preparation” or “cosmetic preparation” are meant to encompass a composition suitable for application or administration to a subject, such as a mammal, especially a human. In general such composition is safe, usually sterile, and preferably free of contaminants that are capable of eliciting an undesirable response of the subject (e.g., the compound(s) in the composition is of an acceptable grade for a given end use). Compositions can be designed for application or administration to subjects or patients in need thereof via a number of different routes of administration including topical, oral, buccal, rectal, parenteral, subcutaneous, intravenous, intraperitoneal, intradermal, intratracheal, intrathecal, pulmonary, and the like. In some embodiments the composition is suitable for application or administration by a transdermal route. In other embodiments, the compositions are suitable for application or administration by a route other than transdermal administration.

The term “derivative” of a compound of the invention include salts, esters, enol ethers, enol esters, acetals, hydrazones, ketals, orthoesters, hemiacetals, hemiketals, acids, bases, solvates, hydrates or prodrugs thereof. Such derivatives may be readily prepared by those of skill in this art using known methods for such derivatization.

The term “salts thereof” of a compound means a salt that possesses the desired activity of the parent compound. Such salts include: (1) acid addition salts, formed with inorganic acids such as hydrochloric acid, hydrobromic acid, sulfuric acid, nitric acid, phosphoric acid, boric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, 1,2,3,4 butane tetracarboxylic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, gluconic acid, glutamic acid, hydroxynaphthoic acid, salicylic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, triethylamine and the like.

The term “solvate or hydrate” of a compound of the invention means a solvate or hydrate complex that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

The term “protecting group” means a chemical group introduced into a molecule by chemical modification of a functional group in order to protect or shield the functional group from its normal chemical reactivity. Protecting groups, their addition and removal are well known (T. W. Greene, P. G. M. Wuts, Protective Groups in Organic Synthesis, Wiley-Interscience, New York, 2005). Removal of the protecting group generates the original functional group, which may be referred to as an “unprotected group”.

The term “prodrug” means any compound that releases an active parent drug in vivo when such prodrug is administered to a mammalian subject. Prodrugs are prepared by modifying functional groups present in the compound in such a way that the modifications may be cleaved in vivo to release the parent compound. Prodrugs include compounds wherein a hydroxyl, amino, carboxyl, or sulfhydryl group is bonded to any group that may be cleaved in vivo to regenerate the free hydroxyl, amino, carboxyl or sulfhydryl group, respectively. Examples of prodrugs include, but are not limited to esters, carbamates, hydrazones, disulfides, and the like.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, fish, and so on. Non-human animal models, particularly mammals, e.g. primate, murine, lagomorpha, etc. may be used for experimental investigations.

As used herein, the terms “determining,” “measuring,” and “assessing,” and “assaying” are used interchangeably and include both quantitative and qualitative determinations.

Throughout the present disclosure, the nomenclature used to describe the amphiphilic lipid compounds is as follows. First, conventional names are used for sterols of the compounds, such as cholesterol, stigmasterol, and sitosterol. For the convenience of description, sterol-modified amphiphilic lipids having glycerol as backbone are abbreviated according to the similar rules for the common names of glycerophospholipids. For example, 1-cholesterylcarbonoyl-2-palmityl-glycero-3-phosphatidylcholine is abbreviated as ChcPePC, wherein: 1) groups at the sn-1/sn-2 positions are represented by capitalized abbreviations (for example, “Ch” for cholesterol, “CHEMS” for cholesteryl hemisuccinyl, “P” for palmitoyl) in the sequence of their substitution positions; 2) the subscript letter or lowercase letter is used to indicate the linkage type such as “c” for carbonate, “e” for ether, “a” for carbamate, and the blank (or no lower case or subscript letter) for ester according to the convention; 3) the head groups are named according to the convention such as “PC” for phosphocholine, and “PG” for phosphoglycerol.

### INTRODUCTION

The present disclosure provides amphiphilic lipid compounds (referred to herein as “sterol-modified amphiphilic lipids” and abbreviated as “SML” or “SMLs”) having two or more hydrophobic tails of which at least one is a sterol. The disclosure also provides methods for the production of such SML compounds and compositions that contain them. The disclosure also provides methods of using SMLs, as well as kits comprising one or more SML compounds and/or compositions.

The compounds and compositions of the invention can be adapted for a variety of applications that find, or benefit by, the use of amphiphilic lipids, for instance, as therapeutic, cosmetic, and imaging agents themselves, to facilitate delivery of therapeutics, cosmetics, detectable labels, and other agents of interest, to detect the presence or absence of an analyte, biosensors and the like. The SMLs can be designed to stabilize water-in-oil or oil-in-water emulsions for use in cosmetic or nutritional products. The SMLs can also be designed so as to act as drugs or prodrugs, as will be described in more detail below.

The compounds of the present disclosure, being amphiphilic in nature, may form a variety of structures alone or with other components in solution, in emulsion, or as a dry powder, and can thus be exploited for many uses. The SML compounds, for instance, can form aggregates, layers, particles, emulsions, structured phases and the like, and can be fine tuned for such purposes. Thus the compounds can be prepared as a composition that comprises one or more of such structures, for instance, as an emulsion or liposome composition, and in particular, as a therapeutic composition, a pharmaceutical composition, a cosmetic composition, and a detection composition.

The SML compounds and compositions have unique properties. One such property is the ability of SMLs to improve the stability of a variety of lipid systems relative to those in which no or a free sterol is used as a stabilizer. For instance, while SMLs can stabilize lipid systems as well as the corresponding free sterol, the SMLs transfer out of the lipid system much, much slower, which in turn improves the stability of the lipid system both in vitro and in vivo (e.g., enhanced resistance to disassociation, degradation or leakage under a variety of physiological and other conditions that typically limit the use of standard lipid systems). Thus lipid systems containing one or more SML compounds provide for more effective applications than the conventional lipid systems in which no stabilizer, or free sterols (e.g., free cholesterol) are used as stabilizers. The SMLs have other advantageous properties as discussed further herein.

One advantage is that the SML compounds can be used alone as an agent of interest, or in combination with other components, such as in the delivery of an agent of interest. For example, the SML itself may comprise a therapeutic, cosmetic or detectable agent. As a therapeutic or cosmetic SML, the hydrophilic head group, and/or one or more of the hydrophilic tail groups of the SML can comprise a therapeutic or cosmetic agent, such as a tail group comprising a sterol comprising a steroid hormone or derivative thereof, and/or a head group comprising a hydrophilic agent, such as carnitine. As detectable SML, the compound can be tagged with a detectable label or exploited as a contrast agent where the SML itself forms micro-bubbles suitable for contrast imaging applications.

Other advantages include the wide variety of applications that can benefit by the use of a stabilized lipid system, as compared to the standard lipid system. For instance, when employed in combination with other components for the delivery of an agent of interest, the SMLs can be provided as a stabilized lipid preparation, such as a stabilized emulsion or liposome preparation.

Examples of applications in lipid systems of particular interest include use of SMLs and compositions as a stable drug carrier for toxic drugs such as doxorubicin, epirubicin, camptothecins, paclitaxel, docetaxel, 5-fluorouracil, cytarabine, cis-platin, tamoxifen, imatinib, irinotecan etc. for parenteral drug administration. Other examples include the use of SMLs and compositions as stable drug carrier for two or more drugs in the same carrier. Other examples include use of SMLs as a stable drug carrier for antimicrobial compounds such as tobramycin for delivery into the lung by inhalation, as drug delivery system for drugs having poor water solubility such as amphoteridin B or retinoic acid. Additional examples include use of SMLs as stable vesicles as an adjuvant and/or vaccine carrier. Additional examples include the use of SMLs as stable carrier of an imaging agent such as a magnetic resonance imaging agent or a radioactive imaging agent. Further examples include use of SMLs as liposomal prodrugs for enzyme-sensitive drug delivery, lipid compositions for reduction-responsive drug delivery, SMLs having sphingosine for the modeling of lipid rafts and the delivery of membrane proteins, SML emulsions having retinoic acid for skin care products, and use of SMLs for micro-bubbles in ultrasound diagnosis. Yet other examples include use of SMLs and compositions as nanoparticles for the delivery of biological macromolecules such as protein, DNA, RNA, siRNA, oligonucleotides, modified nucleic acids or use as nanoparticles for the stabilization of proteins, use of SMLs containing sitosterol or stigmasterol in nutritional products for treatment of hypercholesterolemia, and use of SMLs and compositions as biosensors.

### Sterol-Modified Amphiphilic Lipids

The compounds of the present disclosure are amphiphilic lipid compounds having two or more hydrophobic tails of which at least one is a sterol. The hydrophilic head group is typically linked to two or three hydrophobic tail groups, but may contain more, such as sterol-modified amphiphilic lipids based on a diphosphatidyl glycerol head structure (as with cardiolipin) having four hydrophobic tail groups.

The compounds are designed on the basis of at least two general principles: 1) sterol is covalently attached to a hydrophilic head group; and 2) the compound is an amphiphilic molecule having two or more hydrophobic tails. Accordingly, the SML compounds provide significant flexibility in the design and fine tuning of properties for a given end use, such as: 1) the balance between the overall hydrophobicity of the two tails and the hydrophilicity of the head group; and 2) the desired function of the specific sterol-modified amphiphilic lipid (e.g., use in an emulsion or liposome to facilitate delivery, versus function as a prodrug or as a drug per se, etc.).

The subject compounds may not only help incorporate sufficient sterols in a lipid system to stabilize a system in which the sterol-modified amphiphilic lipid is present, but also inhibit transfer of the sterols from the structure since the sterols are covalently bonded and strongly associated with the system. For instance, sterol-modified amphiphilic lipids in liposomes can stabilize the liposomes and provide for enhanced resistance to leakage of liposome contents under physiological conditions (e.g., 37° C. in the presence of a biological fluid, e.g., in the presence of serum).

With respect to the sterol component of the SML compounds, the sterol group is in general a natural or synthetic structure based on or derived from a sterol compound bearing (or modified to bear) a functional group used for covalent attachment to the hydrophilic head group of the SML. For instance, sterols from biological sources are usually found either as free sterol alcohols, acylated (sterol esters), alkylated (steryl alkyl ethers), sulfated (cholesterol sulfate), or linked to a glycoside moiety (steryl glycosides) which can be itself acylated (acylated sterol glycosides) (See, e.g., Fahy et al., J. Lipid Research (2005) 46:839-861, which reference is incorporated in its entirety). Examples include (1) sterols obtainable from animal sources, referred to herein “zoosterols,” such as the zoosterols cholesterol and certain steroid hormones; and (2) sterols obtainable from plants, fungi and marine sources, referred to herein as “phytosterols,” such as the phytosterols campesterol, sitosterol, stigmasterol, and ergosterol. These sterols generally bear at least one free hydroxyl group, usually at the 3 position of ring A, at another position, or combinations thereof, or can be modified to incorporate a suitable hydroxyl or other functional group as needed. As such, depending of the SML construct, composition and/or given end use, the sterol component can be attached in a SML compound at various positions of the sterol structure.

Sterols of particular interest are the simple sterols, which bear a unique functional group for attachment to the head group of an SML. Of specific interest are simple sterols in which the unique functional group is a hydroxyl, and in particular, the simple sterol alcohols having a hydroxyl group located at position 3 of ring A (e.g., cholesterol, β-sitosterol, stigmasterol, campesterol, and brassicasterol, ergosterol and the like, and derivatives thereof).

In some embodiments, the SML comprises at least one simple sterol. In other embodiments, the SML comprises at least one simple sterol attached to the SML head group at the 3 position of ring A of the sterol. In a specific embodiment, the SML comprises at least one simple sterol attached to the SML head group at the 3 position of ring A of the sterol, and where the sterol such as thiocholesterol, is based on or derived from a simple sterol having a single hydroxyl group located at position 3 of ring A (e.g., cholesterol, β-sitosterol, stigmasterol, campesterol, and brassicasterol, ergosterol and the like, and derivatives thereof).

In certain embodiments, the sterol component of an SML is other than a bile acid or bile salt. In other embodiments, the sterol component of an SML is other than a steroid. In some embodiments, the sterol component of an SML is other than a steroid conjugate. In certain embodiments, the sterol component of an SML is other than a secosteroid.

In other embodiments, the SML compound may include a bile acid component. For instance, one class of SML compounds containing a bile acid component of particular interest are species where the carboxylic group of the bile acid is attached to the amine of the following molecules: D-erythro-sphingosine, sphingosine-1-phosphate, sphingosine phosphorylcholine (lysosphingomyelin), glycosylated sphingosines, sphinganine, sphinganine-1-phosphate, sphinganine phosphorylcholine and the like, where the hydrophobic chain can be altered. A second class of SML compounds containing a bile acid component of interest are those in which a bile acid is attached to one or two hydroxyl groups of glycerol phosphocholine.

In yet other embodiments, the SML compound may include a steroid component, a steroid conjugate component, and/or a secosteroid component that is attached through a suitable linking group to the following molecules: D-erythro-sphingosine, sphingosine-1-phosphate, sphingosine phosphorylcholine (lysosphingomyelin), glycosylated sphingosines, sphinganine, sphinganine-1-phosphate, sphinganine phosphorylcholine and the like, where the hydrophobic chain can be altered, or in other embodiments, to other phospholipid groups.

Exemplary SMLs of specific interest are as follows: SML comprising substituted or unsubstituted cholesterol; SML comprising substituted or unsubstituted β-sitosterol; SML comprising substituted or unsubstituted stigmasterol; SML comprising substituted or unsubstituted campesterol; SML comprising substituted or unsubstituted brassicasterol; and SML comprising substituted or unsubstituted ergosterol.

Cholesterol is of particular interest. Representative sterols of the cholesterol class (including substituted cholesterols) of interest include, for example, the following (See Table 1): (1) natural and synthetic sterols such as cholesterol (ovine wool), cholesterol (plant derived), desmosterol, stigmasterol, β-sitosterol, thiocholesterol, 3-cholesteryl acrylate; (2) A-ring substituted oxysterols such as cholestanol, and cholestenone; (3) B-ring substituted oxysterols such as 7-ketocholesterol, 5α,6α-epoxycholestanol, 5β,6β-epoxycholestanol, and 7-dehydrocholesterol; (4) D-ring substituted oxysterols such as 15-ketocholestene, and 15-ketocholestane; (5) side-chain substituted oxysterols such as 25-hydroxycholesterol, 27-hydroxycholesterol, 24(R/S)-hydroxycholesterol, 24(R/S),25-epoxycholesterol, and 24(S),25-epoxycholesterol; (6) lanosterols such as 24-dihydrolanosterol and lanosterol; (7) fluorinated sterols such as F7-cholesterol, F7-5α,6α-epoxycholestanol, F7-5β,6β-epoxycholestanol, and F7-7-ketocholesterol; (8) fluorescent cholesterol such as 25-NBD cholesterol, dehydroergosterol, and cholesterol triene. These compounds may also include deuterated and non-deuterated versions, and are available commercially, such as from Avanti Polar Lipids, Inc.

TABLE 1
Representative cholesterol and derivative compounds
Natural and synthetic cholesterols 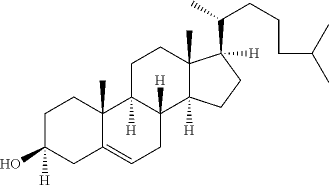
cholesterol (ovine wool or plant derived) 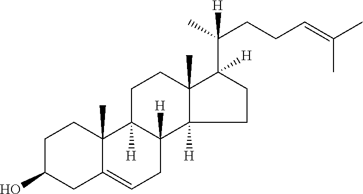
3β-hydroxy-5,24-cholestadiene 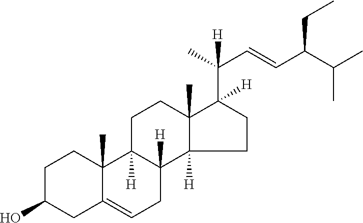
3β-hydroxy-24-ethyl-5,22-cholestadiene 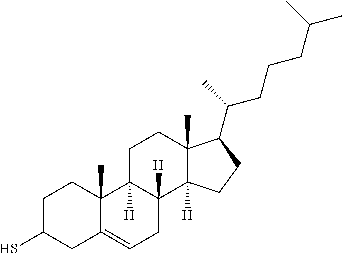
3β-thiocholesterol 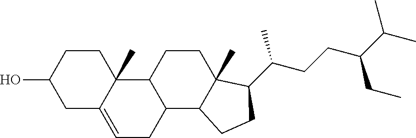
3β-sitosterol 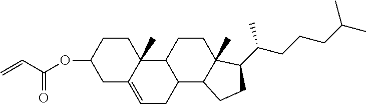
3-cholesteryl acrylate
A-ring substituted oxysterols 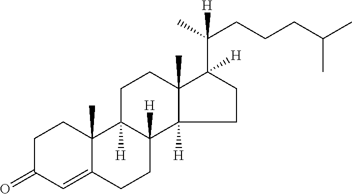
4-cholesten-3-one 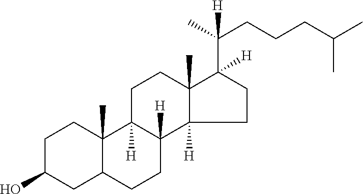
5α-cholestan-3β-ol
B-ring substituted oxysterols 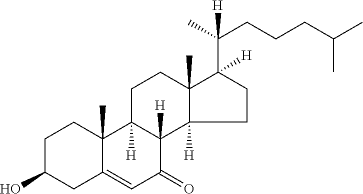
7-ketocholesterol 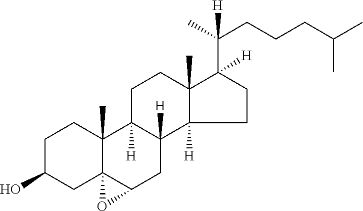
5α,6α-epoxycholestanol 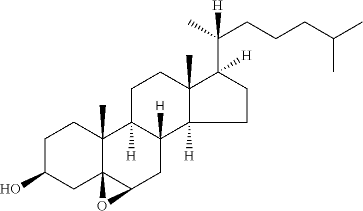
5β,6β-epoxycholestanol 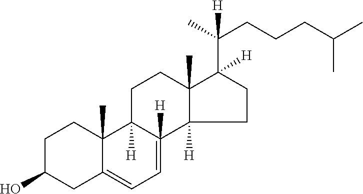
7-dehydrocholesterol
D-ring substituted oxysterols 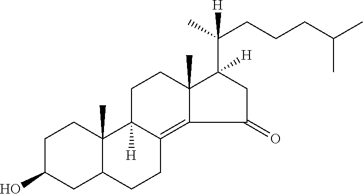
15-ketocholestene 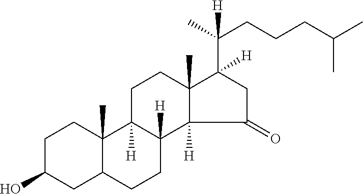
15-ketocholestane
Side-chain substituted oxysterols 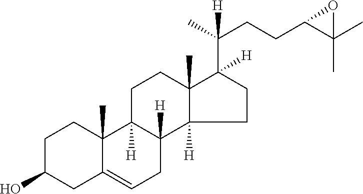
24(S),25-epoxycholesterol 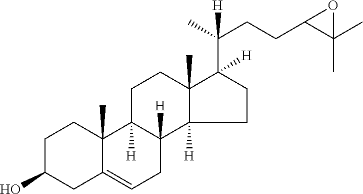
24(R/S),25-epoxycholesterol
Lanosterols 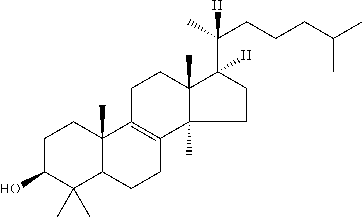
24-dihydrolanosterol (8-lanosten-3β-ol) 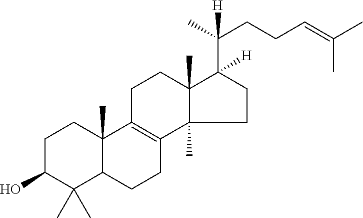
lanosterol (8,24-lanostadien-3-ol)
Fluorinated sterols 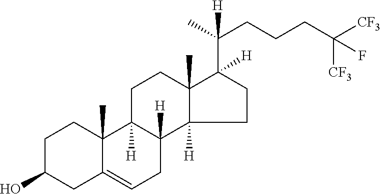
24(R/S),25-epoxycholesterol 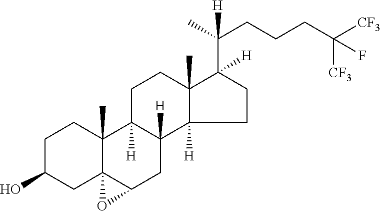
F7-5α,6α-epoxycholestanol 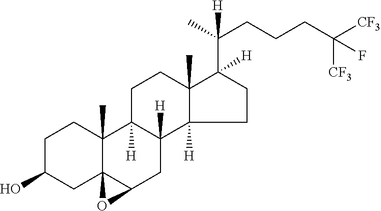
F7-5β,6β-epoxycholestanol 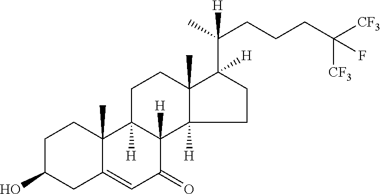
F7-7-ketocholesterol
Fluorescent cholesterol 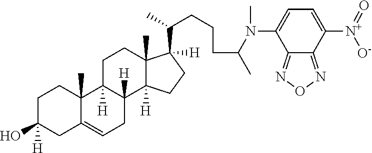
25-NBD cholesterol 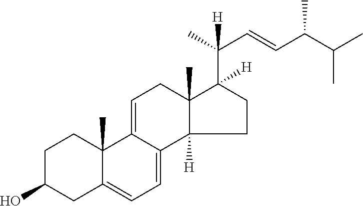
dehydroergosterol (DHE) 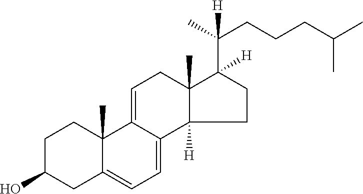
cholesterol triene

The SML compounds may be provided as either racemic or sterically pure compounds. In some embodiments, the SML compounds are stereochemical pure. In other embodiments, a racemic SML is provided. For instance, SMLs comprising lysosphingomyelin and lyso PC provide can be synthesized to provide one isomer (if what is attached is not racemic), whereas other routes provide racemic compounds. In the case of SMLs having an amino acid branching core as part of the head group, these can exist as either D or L forms, or as a racemic compound. Thus, SMLs can be provided in as either the D or L isomer, or as a racemic compound (depending on which form is used in synthesis). Amino acids that are particularly useful as the branching core include: lysine, ornithine, diaminopropionic acid, diaminobutyric acid, diamino acetic acid, aminoethyl glycine, aspartic acid, glutamic acid, cysteine, tyrosine, serine, threonine, histidine, hydroxyl proline, δ′,δ′-bisaminopropyl ornithine, propargyl glycine, and 3,5-amino-benzoic acid.

For the SML compounds containing at least one non-sterol hydrophobic tail, the non-sterol hydrophobic tail(s) comprises a saturated or unsaturated, linear or branched, substituted or unsubstituted an aliphatic chain. Of particular interest is a non-sterol hydrophobic tail comprising about 2 to about 40 carbon atoms in length, and may be saturated or unsaturated, linear or branched, substituted or unsubstituted. For instance, the non-sterol moiety of an SML in this instance is a saturated or unsaturated, linear or branched, substituted or unsubstituted hydrocarbon chain having from 2 to 40 carbon atoms, usually from 4 to 30 carbon atoms, usually from 4 to 25 carbon atoms, more usually from 6 to 24 carbon atoms, more usually from 10 to 20 carbon atoms.

When the non-sterol moiety is a substituted aliphatic hydrocarbon chain that is based on a saturated aliphatic hydrocarbon chain, such as a chain saturated with alkyl groups, then one, two, three, four or more carbon atoms (generally no more than about 10 carbon atoms) of the alkylene groups can be substituted by a heteroatom selected from oxygen, silicon, sulphur or nitrogen atoms, and wherein one, two, three, four or more hydrogen atoms (generally no more than the total number of hydrogen atoms) in the alkylene groups can be substituted with fluoride.

Examples of non-sterol chains of particular interest are those based on or derivable from various lipids, such the aliphatic acids, glycerolipids, glycerophospholipids, sphingolipids, prenol lipids, and saccharolipids, such as the from lipids described in Fahy et al., J. Lipid Research (2005) 46:839-861.

It should be noted that the number of carbons in a hydrocarbon chain of a hydrophobic tail of an SML compound can be selected according to the desired lipophilicity of the molecule. The lipophilicity of the molecule is directly correlated to the selected chain length. Where the sterol-modified amphiphilic lipid is to be used in a liposome, molecules having 10 to 24 carbons are of particular interest, as such provide the molecules suitable hydrophobicity to form stable vesicles. The substitution of carbon atom(s) for alkylene groups in a hydrocarbon chain with a heteroatom such as oxygen can provide for a desired effect on the lipophilicity of the molecule. The same principle applies to the fluorinated hydrocarbon chains. Accordingly, the desired hydrocarbon chain length for one or more of the hydrophobic tails may vary according to the extent of heteroatom substitution. In is also noted that the substituted aliphatic hydrocarbon chains may include those substituted with one or more aryl or heteroaryl groups, such as an aromatic (e.g., phenyl or substituted phenyl) or heteroaromatic (e.g., pyridine or substituted pyridine).

SML compounds also include those in which a hydrophobic tail group can be released from the parent compound through cleavage (e.g., enzymatic or chemical cleavage). For instance, one or more of the covalent bonds in an SML can be capable cleavage under pre-selected conditions, such as an ester linkage exposed to an esterase or alkaline pH conditions. In another example, a disulfide linkage is present in the SML, which can be cleaved under reducing conditions. Of particular interest are SMLs in which at least one of the hydrophobic tails comprises a hydrophobic agent or drug that is linked to the SML through a cleavable bond.

In certain embodiments, one or more of the hydrophobic tail groups of the SML is a polymerizable aliphatic acid, such as 10,12-tricosadiynoic acid. In other embodiments, the SML comprises one or more of the hydrophobic tail groups that comprise (i) a short polypropyleneglycol chain having 6 to 30 carbon atoms; (ii) a silicon-containing linear or branched chain having 3 to 30 silicon atoms; and/or (iii) a prenol lipid having 5 to 40 carbon atoms.

Regarding the hydrophilic head group, this component of the SML compounds comprises a charged group, a polar group, or a combination of charged and polar groups. The charged groups include anionic and cationic moieties. Examples of anionic groups, with the hydrophobic tail and branching core portion of the molecule represented by an R in this context, include but are not limited to, boric acid (RBO₂H₂), carboxylates (RCO₂⁻), sulfates (RSO₄⁻), sulfonates (RSO₃⁻) and phosphates (RPO₄H⁻), phosphonates (RPO₃H⁻) which represent common charged functionalities of the head groups of amphiphilic lipids. Examples of cationic groups include, but are not limited to, amines (RNH₃⁺), methylated amines, polyamines such as spermine and the like, as well as ampholytes that contain both acidic and basic groups (and are therefore amphoteric) that exist as zwitterions or cations at a certain pH (e.g., histidine), which also represent charged functionalities of the head groups of the amphiphilic lipids. The polar, uncharged groups are exemplified by alcohols (—OH), such as glycerols, sugars, polar amino acids (including zwitterionic amino acids), and oligoethyleneglycols. In certain embodiments, the hydrophilic head group comprises other additional elements of an amphiphilic lipid head group, such as choline, ethanolamine, glycerol, nucleic acid, sugar, inositol, azide, propargyl and serine.

The hydrophilic head group may be a natural or synthetic head group, such as an amino acid or derivative, a peptide, a metal chelator, an aryl or heteroaryl derivative, or any suitable structure for attaching the tail groups and bearing a charged or polar property, provided the overall head group is hydrophilic. The hydrophilic head group may also be a structure based on or derived from an amphiphilic lipid bearing (or modified to bear) one or more functional groups used for covalent attachment to the hydrophobic tails of the SML. For instance, the hydrophilic head group may be based on or derived from biological sources of amphiphilic lipids such as glycerolipids, glycerophospholipids, sphingolipids, and saccharolipids, such as the from lipids described in Fahy et al., J. Lipid Research (2005) 46:839-861.

Particular examples of hydrophilic head groups of interest include, but are not limited to a head group comprising a first molecule selected from boric acid (RBO₂H₂), carboxylates (RCO₂⁻), sulfates (RSO₄⁻), sulfonates (RSO₃⁻) and phosphates (RPO₄H⁻), phosphonates (RPO₃H⁻), amines (RNH₃⁺), glycerols, sugars such as lactose or derived from hyaluronic acid, polar amino acids, polyethylene oxides (also known as polyethylene glycol) such as monomethoxypolyethylene glycol, branched polyethylene glycols, and oligoethyleneglycols, that is optionally conjugated to a residue of a second molecule selected from choline, ethanolamine, glycerol, nucleic acid, sugar, inositol, and serine. Here again the head groups may contain various other modifications, for instance, in the case of the oligoethyleneglycols and polyethylene oxide (PEG) head groups, such PEG chain may be terminated with a methyl group or have a distal functional group for further modification.

Examples of hydrophilic head groups of particular interest include, but are not limited to, phosphate, phosphocholine, phosphoglycerol, phosphoethanolamine, phosphoserine, phosphoinositol, ethylphosphosphorylcholine, polyethyleneglycol, polyglycerol, tri-nitrilotriacetic acid, melamine, glucosamine, trimethylamine, spermine, spermidine, and conjugated carboxylates, sulfates, boric acid, sulfonates, sulfates and carbohydrates. Again, the head groups may contain various modifications, such as a polyethyleneglycol head group that is end-functionalized with an activated functional group such as azide, maleimide, bromoacetyl, 2-pyridyldithiol, alkene, or propargyl. The hydrophilic head group may also comprise and/or be conjugated to a residue of a second molecule, with phosphoethanolamine-N-[monomethoxypolyethyleneglycol] 2000 and phosphoethanolamine-N-succinyl-N-tri-nitriloacetic acid representings some examples of specific interest.

Thus in certain embodiments, the SML includes a head group and/or branching core comprising a natural amino acid. In other embodiments, the SML includes a head group and/or branching core comprising a synthetic amino acid. By “natural amino acid” is intended an amino acid obtainable from a biological source, such as the genetically encoded amino acids and other ribosomally installed amino acids occurring in nature. By “synthetic amino acid” is intended an amino acid other than one isolatable from a biological source. As discussed above, examples of such natural and synthetic amino acids include: lysine, ornithine, diaminopropionic acid, diaminobutyric acid, diamino acetic acid, aminoethyl glycine, aspartic acid, glutamic acid, cysteine, tyrosine, serine, threonine, histidine, hydroxyl proline, δ′,δ′-bisaminopropyl ornithine, propargyl glycine, and 3,5-amino-benzoic acid, and can be present in the SML as either the D or L isomer, or as a racemic compound (depending on which form is used in synthesis).

In further embodiments, the SML comprises a hydrophilic head group having a ligand binding moiety, such as a targeting ligand. SMLs adapted with a targeting ligand are capable of selectively forming a high affinity binding pair with a target molecule, such as an antibody or fragment thereof that selectively binds a specific epitope. In one embodiment, the targeting ligand is a metal chelator. Examples of metal chelators include, but are not limited to, ethylenediaminetetraacetic acid (EDTA), diethylenetriaminepentaacetic acid (DPTA), nitrilotriacetic acid (NTA), and derivatives thereof. For instance, tri-nitrilotriacetic acid (tri-NTA) and various derivatives thereof are described in Huang et al., Bioconj. Chem. 2006, 17:1592-1600. A featured SML comprises a head group comprising tri-nitrilotriacetic acid, and derivatives thereof such as phosphoethanolamine-N-succinyl-N-tri-nitriloacetic acid, which SMLs are particularly useful for binding histidine-tagged molecules with high affinity.

Of particular interest are SMLs comprising the formula:

(R¹)(R²)G-X Formula I

where R¹ and R² are hydrophobic tail groups, G is a branching core, and X is a hydrophilic head group, and where at least one of R¹ and R² is a sterol.

The X, G, R¹ and R² groups of Formula I can each individually contribute one or more other features of a compound of Formula I, provided that the compound comprises at least one head group and at least two hydrophobic tails of which at least one is a sterol (e.g., see Formula III). Compounds of Formula I may further include compounds with one or more additional branch points, one or more additional tail, and/or one or more head groups, again provided that the compound comprises at least one hydrophilic head group and at least two hydrophobic tails of which at least one is a sterol. Examples include SML compounds of the formula (R¹)(R²)G-X-G(R³)(R⁴), where R³ and R⁴ may each individually be present or absent, such as with an SML based on a cardiolipin head/branching core construct where R¹, R², R³ and R⁴ are present. Of specific interest are SMLs exemplified by monosterol and disterol amphiphilic lipids of Formula I that have two hydrophobic tails linked to a single head/branching core group.

In one embodiment, where the compound of Formula I contains only one sterol moiety (i.e., a sterol positioned at either R¹ or R²), the non-sterol moiety comprises an aliphatic chain of about 2 to about 40 carbon atoms in length, and may be saturated or unsaturated, linear or branched, substituted or unsubstituted. For instance, the non-sterol moiety (at the corresponding R¹ or R²) is a saturated or unsaturated, linear or branched, substituted or unsubstituted hydrocarbon chain having from 2 to 40 carbon atoms, usually from 4 to 30 carbon atoms, usually from 4 to 25 carbon atoms, more usually from 6 to 24 carbon atoms, more usually from 10 to 20 carbon atoms, wherein one, two, three, four or more carbon atoms (generally no more than about 10 carbon atoms) of the alkylene groups of R¹ or R² can be substituted by a heteroatom selected from oxygen, silicon, sulphur or nitrogen atoms, and wherein one, two, three, four or more hydrogen atoms (generally no more than the total number of hydrogen atoms) in the alkylene groups of R¹ or R² can be substituted with fluoride. Here again the number of carbons and the type and amount of substitution of the non-sterol hydrophobic tail of an SML compound can be selected according to the desired properties of the molecule.

In one embodiment, the monosterol amphiphilic lipids are of Formula I, where one of R¹ or R² is a sterol comprising a zoosterol or a phytosterol (e.g., such as cholesterol, steroid hormones, campesterol, sitosterol, ergosterol, and stigmasterol), and where one of R¹ and R² is a non-sterol moiety comprising a saturated or unsaturated, linear or branched, substituted or unsubstituted hydrocarbon chain having from 2 to 30 carbon atoms, and G is a branching core attaching R¹ and R² to the hydrophilic head group X.

In another embodiment, one of R¹ or R² of Formula I is a saturated or unsaturated, linear or branched, substituted or unsubstituted hydrocarbon chain having from 2 to 40 carbon atoms. In certain embodiments, one of R¹ or R² of Formula I is a saturated or unsaturated, linear or branched, substituted or unsubstituted hydrocarbon chain having from 4 to 24 carbon atoms. In other embodiments, one of R¹ or R² of Formula I is a saturated or unsaturated, linear or branched, substituted or unsubstituted hydrocarbon chain having from 6 to 24 carbon atoms. In yet additional embodiments, one of R¹ or R² of Formula I is a saturated or unsaturated, linear or branched, substituted or unsubstituted hydrocarbon chain having from 10 to 20 carbon atoms.

In general, when one of R¹ or R² of Formula I is based on an unsaturated, substituted aliphatic hydrocarbon chain with one or more alkylene groups, then additional R¹ or R² groups include those in which one, two, three, four or more carbon atoms, but generally no more than about 10 carbon atoms of the alkylene groups can be substituted by a heteroatom selected from oxygen, silicon, sulphur or nitrogen atoms. In addition, one, two, three, four or more hydrogen atoms (generally no more than the total number of hydrogen atoms) in the alkylene groups of an unsaturated R¹ or R² aliphatic hydrocarbon chain can be substituted with fluoride.

Examples of non-sterol chains of particular interest are those based on or derivable from various lipids, such the aliphatic acids, glycerolipids, glycerophospholipids, sphingolipids, prenol lipids, and saccharolipids, such as the from lipids described in Fahy et al., J. Lipid Research (2005) 46:839-861.

In another embodiment, either R¹ or R² of the above compound of Formula I is a hydrophobic drug that may be released from the parent compound through enzymatic cleavage. In another embodiment, according to the above compound of Formula I, R¹, R², and X are linked to G through covalent bonds of which at least one is capable of being cleaved under pre-selected conditions, such as a disulfide linkage exposed to reducing conditions or an ester exposed to alkaline conditions. In another embodiment, either R¹ or R² of the above compound of Formula I is a polymerizable aliphatic acid such as 10,12-tricosadiynoic acid. In another embodiment, either R¹ or R² of the above compound of Formula I is a short polypropyleneglycol chain having 6 to 30 carbon atoms. In another embodiment, either R¹ or R² of the above compound of Formula I is a silicon-containing linear or branched chain having 3 to 30 silicon atoms. In another embodiment, either R¹ or R² of the above compound of Formula I is a prenol lipid having 5 to 40 carbon atoms.

In one embodiment, G of the above compound of Formula I is a branching core having at least three attachment points, where one attachment point is linked to the hydrophilic head group X, and two of the attachment points are each separately linked to the hydrophilic tail groups R¹ and R². Accordingly, in certain embodiments, G is a branching core derived from a compound having at least three functional groups for attachment of R¹, R² and X, such as the compounds selected from the following structures:

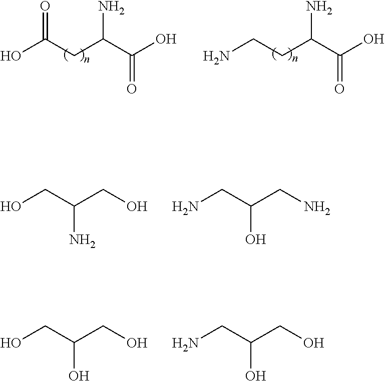

wherein n can be 0, 1, 2, or 3. In this embodiment, R¹ and R² can be at any position, the remaining position is occupied by X. Thus, the hydrophilic head group X is attached to one functional group (e.g., carboxyl (—COOH), amine (—NH₂), or alcohol (—OH)), and the hydrophilic tail groups R¹ and R² are attached to the remaining functional groups (e.g., carboxyl (—COOH), amine (—NH₂), or alcohol (—OH)).

X in Formula I is a hydrophilic group, usually a hydrophilic head group. In a specific embodiment, the hydrophilic group comprises a charged group, a polar group, or a combination of charged and polar groups. Thus the hydrophilic group X (or G-X) may be a synthetic group, such as an amino acid or derivative, an aryl or heteroaryl derivative, or any suitable structure for attaching the tail groups and bearing a charged or polar property, provided the overall head group of the SML is hydrophilic. An example of an aryl or heteroaryl derivative hydrophilic group X of particular interest is chromolyn, and chromolyn glycolate (G-X). The hydrophilic group X (or G-X) may also be a structure based on or derived from an amphiphilic lipid bearing (or modified to bear) one or more functional groups used for covalent attachment to the hydrophobic tails of the SML. For instance, the hydrophilic group X (or G-X) may be based on or derived from biological sources of amphiphilic lipids such as glycerolipids, glycerophospholipids, sphingolipids, and saccharolipids, such as the from lipids described in Fahy et al., J. Lipid Research (2005) 46:839-861. Exemplary groups for X and G-X include, but are not necessarily limited to: phosphate, phosphocholine, phosphoglycerol, phosphoethanolamine, phosphoserine, phosphoinositol, ethylphosphosphorylcholine, polyethyleneglycol, polyglycerol, melamine, glucosamine, hyaluronic acid and trimethylamine.

In some embodiments, G-X is provided by a moiety of interest. For example, as discussed in more detail below, G-X can be provided by carnitine, which is in turn linked to R¹ and R² as per Formula I.

In other embodiments, G-X is provided by diphosphatidylglycerol (e.g., as with cardiolipin), which is in turn linked to R¹ and R² groups (at least two and up to four) as per Formula I.

Exemplary compounds of particular interest are compounds of the general Formula I wherein G is glycerol and X is phosphocholine.

In another embodiment, R¹-G of the above compound of Formula I is selected from sphingosine and sphingonine. In yet another embodiment, G-X of the above compound of Formula I is carnitine.

Of particular interest are sterol glycerophospholipids, which is intended to mean a lipid having a glycerol core with at least one sterol substituted in place of a fatty acid tail group. Examples of sterol glycerophospholipids include any derivative of sn-glycero-3-phosphoric acid that contains at least one sterol residue attached to the glycerol moiety, and a polar head group that is made of, for example, a nitrogenous base, a glycerol, or an inositol unit. For monosterol glycerophospholipids, a sterol is attached to one residue of the glycerol moiety, and another residue of the glycerol moiety is, for example, an O-acyl, O-alkyl, O-alk-1′-enyl or O-carbamate. They can be the same or different subunits of sterols and fatty acids.

In specific exemplary embodiments of the sterol glycerophospholipids, the compound is of Formula I wherein G is glycerol and X is phosphocholine. Compounds of particular interest are described as having the general Formula II:

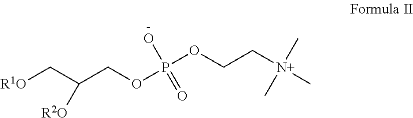

or a pharmaceutically acceptable salt thereof, wherein R¹ and R² are independently a hydrophobic moiety, wherein at least one of R¹ and R² is a sterol, and when only one of R¹ or R² is a sterol, the non-sterol moiety is a saturated or unsaturated, linear or branched, substituted or unsubstituted, hydrocarbon chain having from 2 to 40 carbon atoms, usually from 4 to 25 carbon atoms, more usually from 6 to 24 carbon atoms, more usually from 10 to 20 carbon atoms, wherein one, two, three, four or more carbon atoms of the alkylene groups of R¹ (or R²) can be substituted by a heteroatom selected from oxygen, sulphur or nitrogen atoms, and wherein one, two, three, four or more hydrogen atoms in the alkylene groups of R¹ (or R²) can be substituted with fluoride. The non-sterol moiety of R¹ (or R²) can be any non-sterol moiety as discussed above.

In specific embodiments, the compound of Formula II has a formula as set out in Table 2 below:

TABLE 2
Exemplary Sterol-Modified Phospholipids (SPLs)^{a}

| Lipids | R¹ | R² |
|---|---|---|
| SML1a-c | Chol-OCO | CₙH₂ₙ₊₁NHCO^{b} |
| SML2a-d | Chol-OCO | CₙH₂ₙ₊₁^{c} or C₁₈H₃₅ |
| SML3a-d | Chol | CₙH₂ₙ₊₁CO^{d} or C₁₇H₃₃CO |
| SML4a-d | CₙH₂ₙ₊₁^{e} or C₁₈H₃₅ | Chol-OCO |
| SML5a-d | CₙH₂ₙ₊₁CO^{f} or C₁₇H₃₃CO | Chol-OCO |
| SML6a-d | Chol-OCO, CHEMS^{g}, | Chol-OCO, CHEMS, StigHS, or |
| | StigHS^{h}, or SitoHSⁱ | SitoHS |

^{a}See general formula II for the general structure;
^{b,c,e}n = 18, 16, 14;
^{d,f}n = 17, 15, 13.
^{g}CHEMS: cholesterylhemisuccinic acid;
^{h}StigHS: stigmasterylhemisuccinic acid;
ⁱSitoHS: sitosterylhemisuccinic acid

In further specific embodiments, the compounds of the Formula III, wherein R¹, G and X are contained in sphingosine phosphorylcholine (lysosphingomyelin) and R² is a sterol,

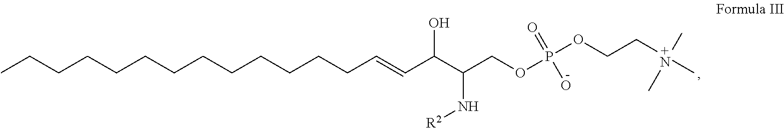

or a pharmaceutically acceptable salt thereof.

An example of a Formula III compound of specific interest is where R² is cholesterol hemisuccinate, or other sterol derivatives.

As reflected in Formula I above, the present disclosure contemplates compounds in which one of R¹ or R² is a sterol, as well as compound in which both R¹ and R² are sterols. In such embodiments, R¹ and R² are selected independently such that they can be the same or different sterols. Examples of particularly useful steroid combinations are between cholesterol and ergosterol, cholesterol and sitosterol, cholesterol and stigmasterol, and stigmasterol and sitosterol.

Exemplary SMLs of specific interested include those based on or derivable from an amphiphilic lipid selected from glycerophospholipids, sphingophospholipids, carnitine and amino acid lipids. For instance, amino acid phospholipids are lipids conjugated to phosphate-modified amino acid hydrophilic head group.

Examples include, but are not limited to:

(1) sterol-modified glycerophospholipids in accordance with Formula II, such as compounds selected from SML1a. SML1b, SML1c, SML2a, SML2b, SML2c, SML2d, SML3a, SML3b, SML3c, SML3d, SML4a, SML4b, SML4c, SML4d, SML5a, SML5b, SML5c, SML5d, SML6a, SML6b, SML6c, SML6d, SML7a, SML7b, SML9a, SML9b, SML9c, SML10a, SML10b, SML10c, SML10d, SML10e, SML10f, SML13a, SML13b, SML13c, SML13d, SML13e, SML13f, SML13g, SML13h, SML15a, SML15b, SML15c, SML15d, SML15e, SML15f, SML15g, SML15h, SML15i, SML15j, SML15k, SML16a, SML16b, SML16c, SML16d, SML16e, SML16f, SML16g, SML16h, SML16i, SML16j, SML16k, SML161, and SML16m, and derivatives thereof;

(2) sterol-modified sphigophospholipids in accordance with Formula III, such as compounds selected from: SML8a, SML8b, SML8c, SML8d, SML8e, and SML8f, and derivatives thereof;

(3) sterol-modified carnitine lipids in accordance with Formula I, such as compounds selected from: SML11a, SML11b, SML11c, SML11d, SML11e, and SML11f, and derivatives thereof;

(4) sterol-modified amino acid lipids in accordance with Formula I, particularly those where the hydrophilic head group comprises an amino acid, such as compounds selected from: SML12a, SML12b, SML12c, SML12d, SML12e, SML12f, SML13i, SML13j, and SML13k, and derivatives thereof; and

(5) sterol-modified amphiphilic lipids in accordance with Formula I having an activated hydrophilic head group that is end-functionalized with an activated moiety such as azide, maleimide, bromoacetyl, 2-pyridyldithiol, alkene, and propargyl, such as a compound selected from: SML14a, SML14b, SML14c, SML14d, SML14e, and SML14f, and derivatives thereof.

As such, featured embodiments include SML compounds selected from: SML1a. SML1b, SML1c, SML2a, SML2b, SML2c, SML2d, SML3a, SML3b, SML3c, SML3d, SML4a, SML4b, SML4c, SML4d, SML5a, SML5b, SML5c, SML5d, SML6a, SML6b, SML6c, SML6d, SML7a, SML7b, SML8a, SML8b, SML8c, SML8d, SML8e, SML8f, SML9a, SML9b, SML9c, SML10a, SML10b, SML10c, SML10d, SML10e, SML10f, SML11a, SML11b, SML11c, SML11d, SML11e, SML11f, SML12a, SML12b, SML12c, SML12d, SML12e, SML12f, SML13a, SML13b, SML13c, SML13d, SML13e, SML13f, SML13g, SML13h, SML13i, SML13j, SML13k, SML14a, SML14b, SML14c, SML14d, SML14e, SML14f, SML15a, SML15b, SML15c, SML15d, SML15e, SML15f, SML15g, SML15h, SML15i, SML15j, SML15k, SML16a, SML16b, SML16c, SML16d, SML16e, SML16f, SML16g, SML16h, SML16i, SML16j, SML16k, SML161, and SML16m, which compounds are depicted in Table 3, and derivatives thereof.

TABLE 3

| | |
|---|---|
| SML1a: R = C₁₈H₃₇ SML1b: R = C₁₆H₃₃ SML1c: R = C₁₄H₂₉ | 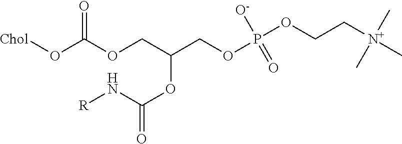 |
| SML2a: R = C₁₈H₃₇ SML2b: R = C₁₆H₃₃ SML2c: R = C₁₄H₂₉ SML2d: R = C₁₈H₃₅ | 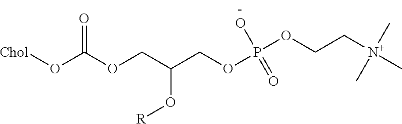 |
| SML3a: R = C₁₇H₃₅ SML3b: R = C₁₅H₃₁ SML3c: R = C₁₃H₂₇ SML3d: R = C₁₇H₃₃ | 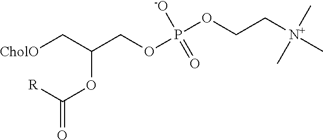 |
| SML4a: R = C₁₈H₃₇ SML4b: R = C₁₆H₃₃ SML4c: R = C₁₄H₂₉ SML4d: R = C₁₈H₃₅ | 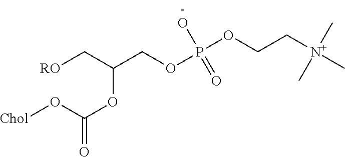 |
| SML5a: R = C₁₇H₃₅ SML5b: R = C₁₅H₃₁ SML5c: R = C₁₃H₂₇ SML5d: R = C₁₇H₃₃ | 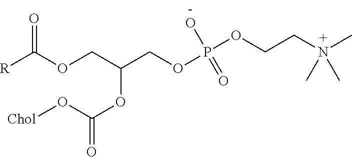 |
| SML6a: RH = cholesterol | 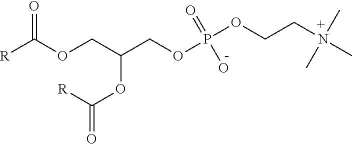 |
| SML6b: RH = cholesterol SML6c: RH = stigmasterol SML6d: RH = β-sitosterol | 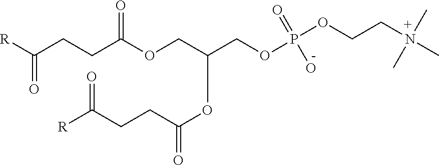 |
| SML7a: RCO₂H = all-trans retinoic acid SML7b: RCO₂H = 13-cis retinoic acid | 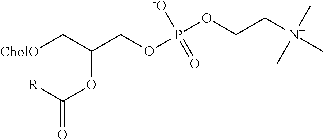 |
| SML8a: R¹H = cholesterol SML8b: R¹H = stigmasterol SML8c: R¹H = β-sitosterol | 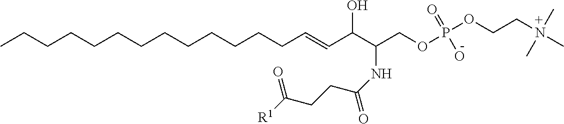 |
| SML8d: R²H = cholesteylcarbonyl SML8e: R²H = cholestryl SML8f: R²H = cholestryloxyoxopropyl | 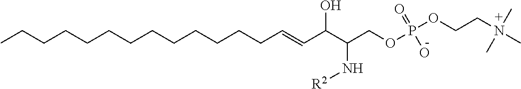 |
| SML9a: RH = cholesterol SML9b: RH = stigmasterol SML9c: RH = b-sitosterol | 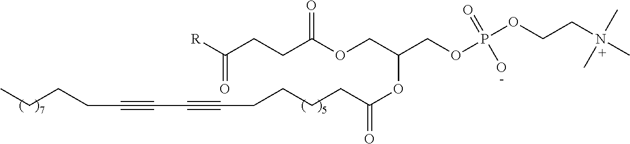 |
| SML10a: R¹OH = iso-Stearic Acid, R²OH = Cholesteryl Hemisuccinate SML10b: R¹OH = iso-Stearic Acid, R²OH = Stigmasteryl Hemisuccinate SML10c: R¹OH = iso-Stearic Acid, R²OH = Sitosteryl Hemisuccinate SML10d: R¹OH = iso-Stearic Acid N, R²OH = Cholesteryl Hemisuccinate SML10e: R¹OH = iso-Stearic Acid N, R²OH = Stigmasteryl Hemisuccinate SML10f: iso-Stearic Acid N, R²OH = Sitosteryl Hemisuccinate | 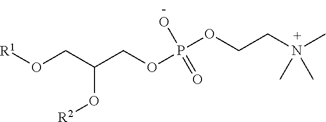 |
| SML11a: R¹ = C₁₇H₃₅, R²OH = cholesterol SML11b: R¹ = C₁₇H₃₅, R²OH = stigmasterol SML11c: R¹ = C₁₇H₃₅, R²OH = sitosterol SML11d: R¹ = C₁₅H₃₁, R²OH = cholesterol SML11e: R¹ = C₁₅H₃₁, R²OH = stigmasterol SML11f: R¹ = C₁₅H₃₁, R²OH = sitosterol | 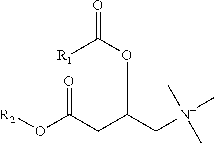 |
| SML12a: R¹ = C₁₈H₃₇, R²OH = cholesterol SML12b: R¹ = C₁₈H₃₇, R²OH = stigmasterol SML12c: R¹ = C₁₈H₃₇, R²OH = sitosterol SML12d: R¹ = C₁₆H₃₃, R²OH = cholesterol SML12e: R¹ = C₁₆H₃₃, R²OH = stigmasterol SML12f: R¹ = C₁₆H₃₃, R²OH = sitosterol | 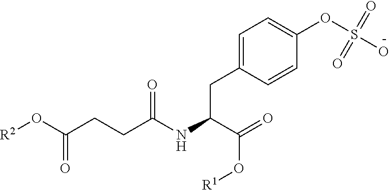 |
| SML13a: R = C₁₈H₃₇ SML13b: R = C₁₆H₃₃ SML13c: R = C₁₄H₂₉ SML13d: R = C₁₈H₃₅ | 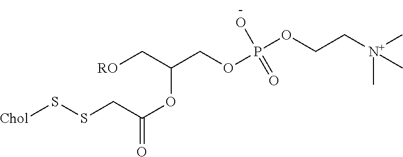 |
| SML13e: R = C₁₇H₃₅ SML13f: R = C₁₅H₃₁ SML13g: R = C₁₃H₂₇ SML13h: R = C₁₇H₃₃ | 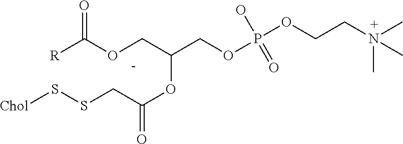 |
| SML13i: R¹H = cholesterol, R² = C₁₇H₃₅ SML13j: R¹H = cholesterol, R² = C₁₅H₃₁ SML13k: R¹H = stigmasterol, R² = C₁₇H₃₅ | 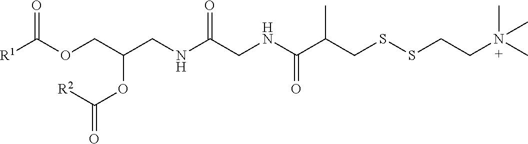 |
| SML14a: R¹H = cholesterol, R² = C₁₇H₃₅ SML14b: R¹H = stigmasterol, R² = C₁₇H₃₅ SML14c: R¹H = sitosterol, R² = C₁₇H₃₅ SML14d: R¹H = cholesterol, R² = C₁₅H₃₁ SML14e: R¹H = stigmasterol, R² = C₁₅H₃₁ SML14f: R¹H = sitosterol, R² = C₁₅H₃₁ | 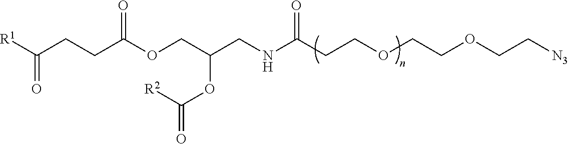 |
| SML15a: R¹ = C₅H₁₁, R²H = cholesterol SML15b: R¹ = C₇H₁₅, R²H = cholesterol SML15c: R¹ = C₉H₁₉, R²H = cholesterol SML15d: R¹ = C₁₁H₂₃, R²H = cholesterol SML15e: R¹ = C₁₃H₂₇, R²H = cholesterol SML15f: R¹ = C₁₅H₃₁, R²H = cholesterol SML15g: R¹ = C₁₇H₃₅, R²H = cholesterol SML15h: R¹ = C₁₇H₃₃, R²H = cholesterol SML15i: R¹ = C₁₉H₃₉, R²H = cholesterol SML15j: R¹ = C₂₁H₄₃, R²H = cholesterol SML15k: R¹ = C₂₃H₄₇, R²H = cholesterol | 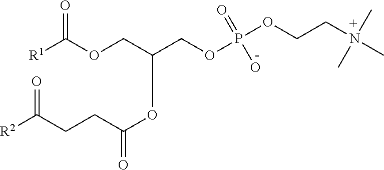 |
| SML16a: R = C₅H₁₁ SML16b: R = C₇H₁₅ SML16c: R = C₉H₁₉ SML16d: R = C₁₁H₂₃ SML16e: R = C₁₃H₂₇ SML16f: R = C₁₅H₃₁ SML16g: R = C₁₇H₃₅ SML16h: R = C₁₇H₃₃ SML16i: R = C₁₉H₃₉ SML16j: R = C₂₁H₄₃ SML16k: R = C₂₃H₄₇ SML16l: R = C₁₅H₃₁ SML16m: R = CH₃(OCH₂CH₂)₈ | 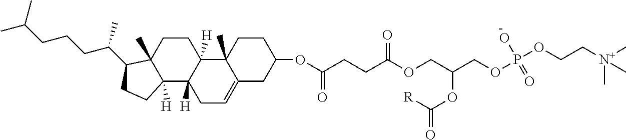 |

### Properties of Sterol-Modified Amphiphilic Lipids

Sterol-modified amphiphilic lipids of the present disclosure can be designed to exhibit a variety of physical properties when present in a bilayer, as in a liposome. Exemplary of such physical properties are phase transition behavior, enthalpy, and resistance to leakage of liposome contents. For example, the stability of liposomes containing SMLs of the present disclosure can be assessed by one or more of contents leakage assays; leakage under physiological conditions (e.g., 37° C. in the presence of serum), or when exposed to an osmotic stress. The effect of the SML on the transition temperature and enthalpy of synthetic diacylphospholipids can also be used to characterize them.

In one embodiment, the sterol-modified amphiphilic lipids provide for stabilized liposomes that are resistant to leakage under physiological, in vivo conditions (or in vitro conditions that model such in vivo physiological conditions) such that less than 30%, less than 25%, less than 20%, less than 15%, less than 10%, or less than 5% to no detectable liposomal content leakage is detected over a period of about 7 days. In further embodiments, the sterol-modified amphiphilic lipids can provide for liposomes that are resistant to leakage under physiological conditions such that less than 25%, less than 20%, less than 15%, less than 10%, or less than 5% or less than 1% to no detectable liposomal content leakage is detected over a period of about 14 days. Furthermore, the sterol-modified amphiphilic lipids can provide for liposomes that are resistant to leakage such that about 80%, 90% or more of the liposomal contents are maintained under physiological conditions for about 7 days; about 60%, 70%, 80%, 90% or more of the liposomal contents are maintained under physiological conditions for about 14 days; about 40%, 50%, 60%, 70%, 80%, 90% or more of the liposomal contents are maintained under physiological conditions for about 21 days; and/or about 20%, 30%, 40%, 50%, 60%, 70%, 80%, 90% or more of the liposomal contents are maintained under physiological conditions for about 28 days.

In a particular embodiment, the stabilized liposome compositions comprise a molar content of a sterol-modified amphiphilic lipid that is usually at least 15%, at least 20%, at least 25%, at least 30%, and can be from 15% to 90%, from 15% to 35%, from 30% to 70%, from 35% to 80%, from 35% to 65%, or from 40% to 70%, and can be present in higher amounts, e.g., from 90% to 95%, or more of the total lipid molar content of the liposome. In specific embodiments, the sterol (e.g., cholesterol) of the sterol-modified amphiphilic lipid is present in the liposome composition so as to provide a molar content of the sterol of at least 25%, at least 30%, at least at least 50%, at least 60%, at least 70%, or more. In a specific embodiment, the liposome compositions comprise a molar content of a monosterol-modified amphiphilic lipid that is at least 30%, at least 35%, at least 70%, at least 85%, or more. In another specific embodiment, the liposome compositions comprise a molar content of a disterol-modified amphiphilic lipid that is from about 15% to 35%, and can be at least 30% or more, at least 40%, at least 45%, or more.

### Sterol-Modified Amphiphilic Lipids as Prodrugs, Drugs, and Desired Properties

The sterol-modified amphiphilic lipids of the present disclosure can be designed so as to provide a desired property, such as a desired physical characteristic (e.g., lipophilicity) and/or functional activity (e.g., activity as a drug or prodrug).

For example, the sterol-modified amphiphilic lipids of the present disclosure can be designed to act as prodrugs, i.e., a compound that can be converted (usually in the body of a subject to whom the compound is administered) from a less active to a more active form. Thus, in one embodiment, either R¹ or R² of the above compound of general Formula I is a hydrophobic drug that can be released from the parent compound through enzymatic cleavage. Thus, for example, general Formula I contemplates compounds wherein R¹ is a drug of interest and R² is sterol. The incorporation of sterol and drug in a single sterol-modified amphiphilic lipid can be advantageous over conventional liposomal prodrugs because a stable liposome may be achieved by the sterol-modified amphiphilic lipid prodrug alone or with minimum complementary components, thus simplifying the prodrug formulation. Additionally, the covalently attached sterol can facilitate stabilization of the liposomal prodrug in biological fluid so that the liposome can gradually accumulate in a targeted therapeutic site, e.g., a site at which cleavage of the prodrug is enhanced relative to other sites in the body (e.g., a site of elevated enzymatic activity, or pH lower or greater than 7.4, etc.).

Where a compound according to the general Formula I is a prodrug, at least one of R1 and R2 is a sterol, and wherein at least one R1 and R2 is a hydrophobic drug. Exemplary hydrophobic drugs include, sterols (such as steroids), carotenoids, vitamins, fatty acids, small molecule hydrophobic drugs, differentiation factors, and anaesthetics. Examples of hydrophobic drugs include, but are not necessarily limited to, retinoic acid (e.g., all trans retinoic acid; 13-cis retinoic acid), steroids and derivatives (e.g., C18 steroids (estrogens) and derivatives; C19 steroids (androgens, such as testosterone and androsterone) and derivatives; C21 steroids (gluco/mineralocorticoids, progestogens as well as the glucocorticoids and mineralocorticoids, and derivatives), and the secosteroids and derivatives (e.g., vitamin D2 and derivatives; vitamin D3 and derivatives, which are characterized by the open B ring of the core structure, hence the “seco” prefix). Of particular interest are prodrug compounds of Formula I where R¹ is a simple sterol, and R² is a hydrophobic drug. In one specific embodiment, R¹ is sterol and R² is retinoic acid. When G-X is phosphocholine, the ester bond at the sn-2 position may be cleaved by phospholipase A2, resulting in the release of retinoic acid. A skilled person in the art will appreciate that many variations and modification can be made in the preparation of SML liposomal prodrugs of desired drugs.

Where desired to provide the sterol-modified amphiphilic lipids as a prodrug, R¹, R², and X are linked to G of Formula I through covalent bonds such that at least one is susceptible to cleavage under a desired condition. For example, the sterol-modified amphiphilic lipid can be enzymatically cleavable, cleavable under reducing conditions, or cleavable under low pH. For example, R¹, R², and X can be linked to G of Formula I through covalent bonds of which at least one is cleavable under reducing conditions. For example, a disulfide bond may be introduced between R² and X so that the cleavage may be triggered by a reducing environment. Compounds of the invention are useful for the triggered release of drugs at specific sites featuring a reducing environment, such as may be found in the intracellular milieu, e.g., in the cytosol, within intracellular vesicles (e.g., endosomes, phagocytic vesicles), and the like.

Sterol-modified amphiphilic lipids can also be designed to have a cationic head group so as to facilitate the delivery of a negatively charged therapeutic moiety through the noncovalent charge-charge interaction. For example, G-X of Formula I can be carnitine, which is an essential molecule in the body's metabolic processes. Compounds of present invention may provide a useful tool for delivery of nucleic acids such as RNA, DNA, oligonucleotides or siRNA into cells in culture and into cells in vivo. For instance, the negatively charged nucleic acid can be complexed by the cationic head group of an SML designed for this purpose and delivered to the target sites.

Sterol-modified amphiphilic lipids can also be designed so that the R¹ and/or R² groups can affect assembly of the compounds, such as in a liposome. For example, in one embodiment, the sterol-modified amphiphilic lipid can comprise a polymerizable chain. For example, either R¹ or R² of the above compound of Formula I contains a polymerizable aliphatic acid, such as 10,12-tricosadiynoic acid. The polymerization of at least one chain of the sterol-modified amphiphilic lipid can endow the molecules with properties such as phase behavior that may be useful for basic biomedical studies, sensor applications or pharmaceutical application.

R¹ or R² can also be selected so as to provide the sterol-modified amphiphilic lipid with a desired lipophilicity. For example, either R¹ or R² of the above compound of Formula I can be a short alkyleneglycol chain, e.g., a polypropyleneglycol chain, having 6 to 30 carbon atoms. The introduction of a short polypropyleneglycol chain in the sterol-modified amphiphilic lipid can affect the hydrophobicity of the molecule and influence assembly of the sterol-modified amphiphilic lipids. A cholesteric liquid crystal phase can be achieved if the structure of the sterol-modified amphiphilic lipids is fine tuned.

Sterol-modified amphiphilic lipids can also be designed so as to function as “lipid rafts”. For example, R² can be a sterol (e.g. cholesterol) and R¹-G of Formula I can be selected from sphingosine and sphingonine. Such compounds can form artificial lipids rafts by interaction between, for example, sphingosine and cholesterol. Artificial lipid rafts can be exploited as a tool for the study of protein-lipid raft interaction, which are thought to play an important role in membrane protein signaling and pathogen entry. These sterol-modified amphiphilic lipids can also find use in delivery of proteins or regulation of the cell membrane domain to prevent the entry of pathogens.

### Methods of Production of Sterol-Modified Amphiphilic Lipids

In another aspect, the present invention provides a variety of processes for the synthesis of the compounds in accordance with the invention. In general, the method comprises coupling at least one sterol tail group through a branching core to a hydrophilic head group so as to generate a sterol-modified amphiphilic lipid having the hydrophilic head group linked through the branching core to two or more hydrophobic tail groups, wherein at least one of the hydrophobic tail groups comprises the sterol tail group.

In a specific embodiment, the method comprises: (i) coupling a branching core to a hydrophilic head group, where the branching core comprises at least one sterol tail group; and (ii) forming a sterol-modified amphiphilic lipid having the hydrophilic head group linked through the branching core to two or more hydrophobic tail groups, wherein at least one of the hydrophobic tail groups comprises the sterol tail group.

In another specific embodiment, the method comprises: (i) coupling at least one sterol tail group to a branching core, where the branching core is linked to a hydrophilic head group; and (ii) forming a sterol-modified amphiphilic lipid having the hydrophilic head group linked through the branching core to two or more hydrophobic tail groups, wherein at least one of the hydrophobic tail groups comprises the sterol tail group.

In the above methods, when the sterol-modified amphiphilic lipid comprises at least one non-sterol tail group, the non-sterol tail group can be attached to the branching core before, concurrent with, or after the sterol tail group is attached.

Thus the methods encompass a variety of different synthesis strategies that provide multiple different routes to the final desired product. For instance, compounds of Formula I can be produced in general by: (i) providing components (a)-(c) as separate, pre-formed components for coupling, where component (a) is branching core G, component (b) is a hydrophobic tail group comprising R¹ and R², and component (c) is a hydrophilic head group X; and then (ii) coupling components (a)-(c) so as to produce a molecule of Formula I. Alternatively, compounds of Formula I can be produced in general by: (i) providing intermediate components (d) and (e) for coupling, where component (d) is branching core G that comprises part of or is linked to hydrophobic tail group components R¹ and R² (i.e., (R¹)(R²)-G′), and component (e) is hydrophilic head group X; and then (ii) coupling components (d) and (e) so as to produce a molecule of Formula I. Standard organic synthesis methods can be employed for such purposes, such as using chemoselective coupling strategies, orthogonal protecting groups and removal etc.

In general, the length of specific synthetic route depends on the complexity of the specific target molecules and the availability of starting materials. For example, disterol phosphocholine may be synthesized in the one step reaction using glycerophosphocholine as the starting material. However, the synthesis of lipids containing ether linkage requires more steps.

In particular, glycerophosphocholine is a useful starting material for the preparation of sterol-modified amphiphilic lipids of general Formula II, but it has poor solubility in most reaction solvents. Thus the present invention provides an efficient method to solubilize glycerophosphocholine in organic solvent by using tetraphenyl borate as the counterion of choline, and specifically as a phase transfer catalyst for the efficient and high yield synthesis of phosphocholine containing amphiphilic lipids in general.

As a specific embodiment, a method is provided for the synthesis of a phosphocholine containing amphiphilic lipid, the method comprising: (i) complexing a phosphocholine compound having at least one functional group with tetraphenyl borate in organic solvent, and (ii) coupling one or more lipids to the functional group of the phosphocholine compound, where the lipid comprises at least one functional group that is capable of reacting with and coupling to functional group of the phosphocholine compound. In this aspect of the invention, the organic solvent, functionalized phosphocholine compound and functionalized lipid(s) are chosen for compatibility with the reaction. The solvents and other agents for coupling are standard (e.g., methanol, pyridine, 4,4-dimethylaminopyridine, and the like). The functional groups can be any that are chemoselective in the reaction, such as chloroformate esters of lipids and hydroxyls of a functional group on the phosphocholine compound. Examples of functionalized phosphocholines include, but are not limited to glycerophosphocholine, amino acid functionalized phosphocholine and the like. In certain embodiments, various protecting group strategies may be exploited for single lipid attachment schemes, as well as orthogonal attachment of different lipids. The method may further include the step of (iii) purifying the phosphocholine containing amphiphilic lipid.

The invention also provides a method for the production of a composition comprising a sterol-modified amphiphilic lipid. This method involves admixing a sterol-modified amphiphilic lipid with at least one of a non-sterol amphiphilic lipid, a therapeutic agent, a cosmetic agent, a detectable label, a buffer, a solvent, and an excipient. This method may further comprise purifying the composition. In general, methods of producing lipid containing compositions are well, known, and thus can be exploited in the production of the SML compositions of the invention. The method is particularly useful in the production of liposomes and emulsions.

Thus in a specific embodiment, the invention provides a method of forming a liposome comprising a sterol-modified amphiphilic lipid compound of the invention. In general, the method involves subjecting a sterol-modified amphiphilic lipid to liposome forming conditions, whereby a liposome is formed. The liposome forming conditions are typically the standard conditions well known in the liposome art. The method may optionally include admixing the sterol-modified amphiphilic lipid with one or more other amphiphilic lipids, agents, buffers, solvents, and/or excipients, and subjecting the mixture to liposome forming conditions. The method also may further include the step of purifying the liposomes by various well known methods, such as by chromatography, phase separation, solvent extraction, lyophilization, re-hydration and the like.

In certain embodiments, the sterol-modified amphiphilic lipid content of a liposome of the invention ranges from at least 1% up to 100%, which specifically includes ranges therein between that are in fractional increments, such as 0.5%, 1%, 1.5%, 2%, 2.5%, 3%, 3.5%, 4%, 4.5% and 5% increments, for example, in 5% increments where the sterol-modified amphiphilic lipid content is selected from 5%, 10%, 15%, 20%, 25%, 30%, 40%, 45%, 50%, 55%, 60%, 65%, 70%, 75%, 80%, 85%, 90%, 95% and 100%.

In a specific embodiment, the method of producing a liposome comprises forming a liposome by admixing under liposome forming conditions (i) one or more amphiphilic lipids with (ii) one or more sterol-modified amphiphilic lipids, wherein the sterol-modified amphiphilic lipid comprises a head group linked through a branching core to two or more hydrophobic tail groups, and wherein at least one of the hydrophobic tail groups comprises a sterol.

In a specific embodiment, the amphiphilic lipid and the sterol-modified amphiphilic lipid are admixed in a molar ratio so as to provide a liposome composition having a molar percentage of the sterol-modified amphiphilic lipid that is at least 1%. Thus in certain embodiments, the amphiphilic lipid and the sterol-modified amphiphilic lipid are admixed in a molar ratio so as to provide a liposome composition having a molar sterol-modified amphiphilic lipid content that ranges from at least 1% to less than 100%, which specifically includes ranges therein between that are in fractional increments, such as 0.5%, 1%, 1.5%, 2%, 2.5%, 3%, 3.5%, 4%, 4.5% and 5% increments, for example, in 5% increments where the sterol-modified amphiphilic lipid content is selected from 5%, 10%, 15%, 20%, 25%, 30%, 40%, 45%, 50%, 55%, 60%, 65%, 70%, 75%, 80%, 85%, 90%, 95%.

A featured aspect of the invention is a liposome composition having a molar content of a sterol-modified amphiphilic lipid that is usually at least 15%, at least 20%, at least 25%, at least 30%, and can be from 15% to 90%, from 15% to 35%, from 30% to 70%, from 35% to 80%, from 35% to 65%, or from 40% to 70%, and can be present in higher amounts, e.g., from 90% to 95%, or more of the total lipid molar content of the liposome. In specific embodiments, the sterol (e.g., cholesterol) of the sterol-modified amphiphilic lipid is present in the liposome composition so as to provide a molar content of the sterol of at least 25%, at least 30%, at least at least 50%, at least 60%, at least 70%, or more. In a specific embodiment, the liposome compositions comprise a molar content of a monosterol-modified amphiphilic lipid that is at least 30%, at least 35%, at least 70%, at least 85%, or more. In another specific embodiments, the liposome compositions comprise a molar content of a disterol-modified amphiphilic lipid that is from about 15% to 35%, and can be at least 30% or more, at least 40%, at least 45%, or more.

In another embodiment, the invention provides a method of forming an emulsion comprising a sterol-modified amphiphilic lipid compound of the invention. In general, the method involves subjecting a sterol-modified amphiphilic lipid to emulsion forming conditions, whereby an emulsion is formed. The method can be used to produce oil-in water type SML emulsions, or water-in oil type SML emulsions and the like. For example, the methods may be exploited for the construction of SML micelles (e.g., oil-in-water systems), as well as reverse or inverse SML micelles (e.g., water-in-oil systems).

The emulsion forming conditions are typically the standard conditions well known in the emulsion art. For instance, the emulsion forming conditions comprises dispersing a SML in an aqueous continuous phase, whereby a water-in-oil emulsion of the SML is produced. Alternatively, the emulsion forming conditions comprise dispersing an aqueous phase in continuous phase of SML, whereby an oil-in water emulsion of the SML is produced.

The emulsion forming method may optionally include admixing the sterol-modified amphiphilic lipid with one or more other amphiphilic lipids, agents, buffers, solvents, and/or excipients, and subjecting the mixture to emulsion forming conditions. The method also may further include the step of purifying the emulsion by various well known methods.

Emulsification can be aided by shaking, stirring, homogenizing, or spray processes as needed to form the emulsion. The SML may be used as a surfactant or as an emulsifier in other emulsions, for example, to stabilize the emulsion for storage, and in particular for use in the preparation of emulsions with therapeutics, cosmetics, and pharmaceuticals (e.g., formulations, creams and lotions).

Whether an emulsion turns into a water-in-oil emulsion or an oil-in-water emulsion depends in part on the volume fraction of both phases and on the type of emulsifier. Generally, the Bancroft rule applies: emulsifiers and emulsifying particles tend to promote dispersion of the phase in which they do not dissolve very well.

In certain embodiments, nanoemulsions are provided, in which the sizes of the particles in the dispersed phase are less than 1000 nanometers.

### Compositions Comprising Sterol-Modified Amphiphilic Lipids

The present disclosure contemplates a variety of compositions containing sterol-modified amphiphilic lipids of the present disclosure. Such compositions can be homogenous with respect to the sterol-modified amphiphilic lipid compound, or can include one or more of the different sterol-modified amphiphilic lipid compounds disclosed herein. Compositions having mixtures of different sterol-modified amphiphilic lipids, e.g., different sterol-modified amphiphilic phospholipids, can provide for fine tuning of the physical properties of the compositions, particularly where the composition is a liposome.

For example, the relative amounts of an amphiphilic lipid and of sterol-modified amphiphilic lipid (e.g., monosterol-modified amphiphilic lipids (or “m-SML”), disterol-modified amphiphilic lipids (or “d-SML”), or combinations of both) can be varied to provide for a desired physical property such as, for example, phase transition temperature, resistance of a liposome to leakage under physiological conditions, storage stability (e.g., at about 4° C.) for a desired period of time (e.g., at least one week, at least one month, at least one year, and the like), and the like. In general a “stable formulation” is one that retains about 90% of encapsulated contents over a defined period.

Thus in certain embodiments, the sterol-modified amphiphilic lipid content of a lipid-containing composition (e.g., liposome or emulsion) can range from at least 1% up to 100%, which specifically includes ranges therein between that are in fractional increments, such as 0.5%, 1%, 1.5%, 2%, 2.5%, 3%, 3.5%, 4%, 4.5% and 5% increments, for example, in 5% increments where the sterol-modified amphiphilic lipid content is selected from 5%, 10%, 15%, 20%, 25%, 30%, 40%, 45%, 50%, 55%, 60%, 65%, 70%, 75%, 80%, 85%, 90%, 95% and 100%. In exemplary embodiments, lipid-containing compositions contain a molar content of a sterol-modified amphiphilic lipid that is usually at least 15%, at least 20%, at least 25%, at least 30%, and can be from 15% to 90%, from 15% to 35%, from 30% to 70%, from 35% to 80%, from 35% to 65%, or from 40% to 70%, and can be present in higher amounts, e.g., from 90% to 95%, or more of the total lipid molar content of the liposome. In specific embodiments, the sterol (e.g., cholesterol) of the sterol-modified amphiphilic lipid is present in the liposome composition so as to provide a molar content of the sterol of at least 25%, at least 30%, at least at least 50%, at least 60%, at least 70%, or more. In a specific embodiment, the liposome compositions comprise a molar content of a monosterol-modified amphiphilic lipid that is at least 30%, at least 35%, at least 70%, at least 85%, or more. In another specific embodiment, the liposome compositions comprise a molar content of a disterol-modified amphiphilic lipid that is from about 15% to 35%, and can be at least 30% or more, at least 40%, at least 45%, or more.

Lipid-containing compositions contemplated also include those having a molar ratio of non-sterol modified amphiphilic lipid and sterol-modified amphiphilic lipid so as to provide a lipid-containing composition having a sterol-modified amphiphilic lipid content that is at least 1%. Thus in certain embodiments, for example a liposome, the non-sterol modified amphiphilic lipid and the sterol-modified amphiphilic lipid are present in a lipid-containing composition admixed in a molar ratio so as to provide a liposome composition having a sterol-modified amphiphilic lipid content that ranges from at least 1% to less than 100%, which specifically includes ranges therein between that are in fractional increments, such as 0.5%, 1%, 1.5%, 2%, 2.5%, 3%, 3.5%, 4%, 4.5% and 5% increments, for example, in 5% increments where the sterol-modified amphiphilic lipid content is selected from 5%, 10%, 15%, 20%, 25%, 30%, 40%, 45%, 50%, 55%, 60%, 65%, 70%, 75%, 80%, 85%, 90%, 95%.

The non-sterol amphiphilic lipid in SML-containing compositions can be any suitable amphiphilic lipid (including any of a variety of conventional lipids, including commercially available lipids), such as non-sterol modified amphiphilic lipids having aliphatic hydrocarbon chains that are saturated or unsaturated, linear or branched, and/or substituted or unsubstituted. Where the SML is a monosterol-modified amphiphilic lipid, it may be desirable to select the m-SML and the non-sterol amphiphilic lipid in the composition so that the hydrophobic tail groups of the non-sterol amphiphilic lipid and the m-SML are similar in character, e.g., the hydrophobic tail groups of the non-sterol amphiphilic lipid and the m-SML are of approximately the same length, are similarly substituted, and the like.

The aliphatic hydrocarbon chains of the non-sterol amphiphilic lipids can be of any a variety of different chain lengths, e.g., from 2 to about 40 carbon atoms in length, and may be saturated or unsaturated, linear or branched, substituted or unsubstituted. For example, the non-sterol moiety in this instance is a saturated or unsaturated, linear or branched, substituted or unsubstituted hydrocarbon chain having from 2 to 40 carbon atoms, usually from 4 to 30 carbon atoms, usually from 4 to 25 carbon atoms, more usually from 6 to 24 carbon atoms, more usually from 10 to 20 carbon atoms. When the non-sterol moiety is a substituted aliphatic hydrocarbon chain that is based on a saturated aliphatic hydrocarbon chain, such as a chain saturated with alkenyl groups, then one, two, three, four or more carbon atoms (generally no more than about 10 carbon atoms) of the alkylene groups can be substituted by a heteroatom selected from oxygen, sulfur or nitrogen atoms, and wherein one, two, three, four or more hydrogen atoms (generally no more than the total number of hydrogen atoms) in the alkylene groups can be substituted with fluoride.

Where the composition is to be administered to a subject, it is generally desirable that the composition be sterile and can be store in a sterile container (e.g., a sterile vial). Compositions can be formulated for a variety of different routes of administration including, parenteral, enteral, nasal, and pulmonary administration, and can include one or more excipients. Exemplary formulations include topical, injectable, aerosol, and oral formulations, and can be formulated as pharmaceutical preparations, cosmeceutical preparations, nutriceutical preparations, and the like. Compositions containing sterol-modified amphiphilic lipids can be lyophilized, stored as dry powders, or may be stored in solution.

### Lipid Particles Comprising Sterol-Modified Amphiphilic Lipids

The SML-containing compositions of the present disclosure can be provided in a variety of different forms, which are generally referred to herein as lipid particles. “Lipid particle” as used herein is mean to encompass SML-containing particles of defined or undefined structure. As amphiphilic molecules are composed of a hydrophilic and hydrophobic segments, in aqueous environments, the SML head groups face toward the water while their hydrophobic tail groups interact with each other to create a lamellar bilayer, and to a lesser extent other aggregate structures depending on the lipid composition and conditions. Thus the SML compounds can form a variety of different shapes including spheres (vesicles), rods (tubes) and lamellae (plates) depending on lipid and water content, and temperature. These shapes represent basic units that interact to form two- and three-dimensional lattice matrix structures classified as lamellar phase (e.g., bilayer plate, closed sphere), hexagonal phase (e.g., rod), or cubic phase (e.g., spheres, rods or lamellae connected by solvent channels).

The most favorable way to organize the segments when the molecules are dispersed in a solvent is to form structures where the hydrophobic and hydrophilic parts are separated into different domains. These domains and the structures arising from the organized domains, are termed solvent-induced liquid crystalline phases. Examples of such phases are: micellar, lamellar, hexagonal, cubic and sponge phases. Phases can be both normal and inverted. In the former case, the interface is curved towards the oil and in the inverted case, the interface is curved towards water. The type of phase depends upon both global parameters, such as the water to oil ratio of the mixture, and on more specific properties of the amphiphilic molecule. In the hexagonal phase the amphiphilic molecules are aggregated into cylindrical structures of indefinite length and these cylindrical aggregates are disposed on a hexagonal lattice, giving the phase long-range orientational order. Bicontinuous cubic phases can also exist. The more important phases for drug delivery are the micellar, cubic and lamellar. The amphiphilic molecules described herein are suitable for being part of or forming one or more of these phases.

Exemplary lipid particles include liposomes (e.g., multi-vesicular, multi-lamellar liposomes, pauci-lameller liposomes, uni-lamellar liposomes, and the like), emulsions (including oil and water emulsions, e.g., oil-in-water emulsions, water-in-oil emulsions, and the like, where the oil can be, for example, a triglyceride), solid core emulsions, lipid aggregates (e.g., hexagonal, cubic phase), lipid monolayers (e.g., as may be present on a surface), lipid foams, and the like.

Sterol-modified amphiphilic lipid-containing liposomes and emulsions, which may further optionally comprise an agent of interest as a payload, are of particular interests. The term “liposome” encompasses any compartment enclosed by a lipid bilayer system. “Liposomes”, which can also be referred to as lipid vesicles, is meant to encompass various forms of liposomes, such as multilamellar liposomes (which generally have an average diameter in the range of 0.5 to 10 micrometers and are comprised of anywhere from two to hundreds of concentric lipid bilayers alternating with layers of an aqueous phase), unilamellar vesicles (which are generally comprised of a single lipid layer and generally have an average diameter in the range of about 20 to about 400 nanometers (nm), about 50 to about 300 nm, about 300 to about 400 nm, about 100 to about 200 nm), and other vesicle forms, such as pauci-lamellar vesicles or multivesicular liposomes.

### Methods of Preparing Compositions Having Sterol-Modified Amphiphilic Lipids

The disclosure also provided methods for producing a composition comprising a sterol-modified amphiphilic lipid. In general, such methods involve admixing a sterol-modified amphiphilic lipid with at least one of a non-sterol amphiphilic lipid and, optionally, loading the composition with a payload which can be, for example, a therapeutic agent (e.g., pharmaceutical agent, nutriceutical agent), a cosmetic agent, a detectable agent (e.g., an imaging agent). The compositions can be admixed in the presence of one or more of a buffer, a solvent, and an excipient. The method also may further include the steps of sizing the liposomes, which can be performed prior or after loading the liposome with payload. Optionally, the methods can include purifying the liposomes by various well known methods, such as by chromatography, phase separation, solvent extraction, lyophilization, re-hydration and the like. Compositions made by these methods can be purified so that the compositions is at least 60% free, usually at least 75% free, and most usually at least 90% free from impurities.

These methods include production of lipid-containing compositions that have a molar ratio of amphiphilic lipid and sterol-modified amphiphilic lipid so as to provide a lipid-containing composition having a sterol-modified amphiphilic lipid content of the compositions described above.

Compositions containing one or more sterol-modified amphiphilic lipid particles can be readily formed by available techniques. For example, such compositions can be readily formed by placing lipids which will include sterol-modified amphiphilic lipids in an aqueous solution and agitating the solution for a period of time of several seconds to hours. The simple procedure spontaneously yields large, multilamellar liposomes or vesicles with diameters in the range of about 1 to 10 micrometers. These liposomes are comprised of two to several hundred concentric lipid bilayers which may alternate with layers of the aqueous phase which the lipids were present within. Further exemplary methods of making liposomes are provided below.

Where it is desired to provide compositions containing liposomes with an encapsulated payload (e.g., an encapsulated agent, a therapeutic agent, imaging agent, or the like, such agents can be included within the aqueous phase with an encapsulating amount of one or more sterol-modified amphiphilic lipids, e.g., one or more sterol-modified amphiphilic phospholipids. Alternatively, where the agent of interest is hydrophobic and thus less soluble in water, such agents can be included within the lipid bilayer. The term “encapsulating amount” refers an amount of a lipid required to encapsulate an agent of interest and form liposomes of a desired size. In general, the average liposome size is less than 10,000 nm in diameter, more usually less than 5,000 nm, and still more usually about 20-600 nm. The encapsulating amount will depend upon the particular compounds and process conditions selected, but will in general range from about 2:1 to about 1:100 compound:lipid, usually about 1:1 to about 1:20. Alternatively, a remote control loading method can be used to introduce the payload into the liposome. In contrast to the loading methods described above, remote loading involves first producing the liposomes, then introducing the payload into the liposomes by means of an ion-gradient.

Incorporation of Payload

The sterol-modified amphiphilic lipid-containing compositions can include a payload for transport with the lipid particle (e.g., with the liposome). “Payload” refers to a component of the sterol-modified amphiphilic lipid-containing composition that is transported with and delivered by the SML-containing composition. Representative payloads include a component that is contained within the structure of a lipid particle (e.g., within a liposome), present in a bilayer or monolayer of a lipid particle, as part of the SML lipid itself (e.g., SML prodrug), or attached to a surface of a lipid particle (e.g. by covalent or non-covalent bonds). Thus a “payload” can include components that are encapsulated by the lipid particle (e.g., pharmaceutical agents, nutraceutical agents, cosmeceutical agents, imaging agents (e.g., gases, including air), radiopharmaceuticals, nuclear magnetic resonance contrast reagents, and the like). In certain embodiments, the encapsulated payload is typically in solution, as a crystal, as a powder, or a combination thereof. In some embodiments, the payload is a component of the sterol-modified amphiphilic lipid that can be released by cleavage of a cleavable bond present in the compound (e.g., where the sterol-modified amphiphilic lipid acts as a prodrug). In some embodiments the payload may be a virus (e.g., an inactivated or attenuated virus) or bacteria (e.g., an inactivated or attenuated bacteria) or nucleic acid.

Methods for preparing drug-containing liposome suspensions generally follow conventional liposome preparation methods. In one exemplary method, sterol-modified amphiphilic lipids are taken up in a suitable organic solvent or solvent system, and dried in vacuo or under an inert gas to a lipid film. Lipophilic agents of interest can be included in the lipids forming the film. The payload that is to be encapsulated in the lipid particle and/or within the lipid particle bilayer can be provided in the lipid solution (e.g., in molar excess of the final maximum desired concentration of the agent in the liposome) so as to facilitate maximum entrapment. Alternatively, where the agent of interest is water-soluble (e.g., more hydrophilic), the agent can be included in the aqueous medium used to hydrate the lipid. Alternatively, the lipid particles (e.g., liposomes) can be generated first, then the payload introduced into the particles by a remote loading method, e.g., by providing an ion-gradient (e.g., an ammonium sulfate gradient) that facilitates movement of an agent of interest into the liposome.

Attachment of a payload to a surface of a lipid particle can be accomplished by, for example, covalent attachment to the distal functional group of a sterol-modified amphiphilic lipid of the lipid particle (Example 14). Various methods suitable for use or that can be adapted for use for attachment of a payload are described in, for example, Liposomes: 2nd edition, Oxford University Press, 2003, V. Torchilin and V. Weissig., Ed.

Sizing

The liposome suspension may be sized to achieve a desired size distribution of vesicles in a size range less than about 1 micron and usually between about 0.05 to 0.5 microns, and most usually between about 0.05 and 0.2 microns. The sizing serves to eliminate larger liposomes and to produce a defined size range having optimal pharmacokinetic properties.

Several techniques are available for reducing the sizes and size heterogeneity of liposomes. Sonicating a liposome suspension either by bath or probe sonication produces a progressive size reduction down to small unilamellar vesicles (SUVs) less than about 0.05 microns in size. Homogenization is another method which relies on shearing energy to fragment large liposomes into smaller ones. In a typical homogenization procedure, MLVs are recirculated through a standard emulsion homogenizer until selected liposome sizes, typically between about 0.1 and 0.5 microns, are observed. Processing the lipid dispersion in the presence of hard spherical beads in a vial placed in a dual asymmetric centrifugate can reduce liposome particle diameter to between 0.04 and 0.3 microns. In these three methods, the particle size distribution can be monitored by conventional laser-beam particle size discrimination.

Extrusion of liposomes through a small-pore polycarbonate membrane is an effective method for reducing liposome sizes down to a relatively well-defined size distribution whose average is in the range between about 0.05 and 8 micron, depending on the pore size of the membrane. Typically, the suspension is cycled through the membrane several times until the desired liposome size distribution is achieved. The liposomes may be extruded through successively smaller-pore membranes, to achieve a gradual reduction in liposome size.

Centrifugation and molecular sieve chromatography are other methods which are available for producing a liposome suspension with particle sizes below a selected threshold less than 1 micron: These two methods both involve removal of larger liposomes, rather than conversion of large particles to smaller ones. Liposome yields are correspondingly reduced.

Removing Free Payload Material

Unincorporated payload, or “free” payload, can be removed, e.g., to increase the ratio of liposome-entrapped payload present in the composition. The removal can be designed to reduce the final concentration of free agent to less than about 20% and, usually less than about 10% of the total payload (e.g., total drug) present in the composition.

Several methods are available for removing free payload from a liposome suspension. Sized liposome suspension can be pelleted by high-speed centrifugation, leaving free payload and very small liposomes in the supernatant. Another method involves concentrating the suspension by ultrafiltration; then resuspending the concentrated liposomes in an agent-free replacement medium. Alternatively, gel filtration can be used to separate larger liposome particles from solute molecules.

One exemplary procedure for removing free payload utilizes an ion-exchange resin capable of binding the agent in free, but not in liposome-bound, form. Selection of a cation-exchange or anion-exchange resin can be based on the charge of the free agent at, for example, neutral pH.

Payload of Sterol-Modified Amphiphilic Lipid-Containing Compositions

Sterol-modified amphiphilic lipid-containing compositions can be exploited so as to facilitate delivery of a payload associated with the composition. As noted above, “payload” refers to a component of the sterol-modified amphiphilic lipid-containing composition that is transported with the sterol-modified amphiphilic lipid-containing composition, and thus can encompass for example, a component that is contained within the structure of a lipid particle (e.g., within a liposome), present in a bilayer of a lipid particle, or attached to a surface of a lipid particle (e.g. by covalent or non-covalent bonds).

The payload may thus be any of a variety of different agents, which may be adapted for a variety of different uses including, but not limited to pharmaceutical, nutriceutical, cosmeceutical, and diagnostic applications. Exemplary agent include, but are not limited to, bisphosphonates, carboplatin, cisplatin, oxaloplatin, carmustine, camptothecins, ciprofloxacin, chloromethane, cyclophosphamide, cyclopamine, cytosine arabinoside, dacarbazine, retinoic acid, doxifluridine, fluoroortic acid, geldanamycin, gemcitabine, gossypol, ifosfamide, hydroxytamoxifen, inrinotecan, phytic acid, protein kinase inhibitors, paclitaxel, resveratrol, taxanes, methylselano-cysteine, methotrexate, 6-thioguanine, tyrphostin, wogonin, etoposide, antisense oligonucleotides, siRNA, chemically modified RNA, citrate, 1, 2, 3, 4 butane tetracarboxylic acid, octasulfate sucrose, polyphosphates, ciprofloxicin, morphine, oxymorphone, buprenorphine and methadone. Such agents can be encapsulated alone or with another agent in the same liposome (e.g., one or more agents, two or more agents) to provide synergistic effects.

Payloads of particular interest include, but are not limited to, anti-cancer chemotherapeutics (e.g., doxorubicin, danorubicin, camptothecin, cisplatin, and the like), antibiotics (e.g., antibacterials, antifungals, antivirals, anti-parasitic agents, and the like), analgesics, anesthetics, anti-acne agents, biomolecules (e.g., nucleic acids (e.g., RNA, DNA, siRNA, and the like), polypeptides (e.g., peptides, including recombinant polypeptides and peptides, including naturally or chemically modified polypeptides and peptides (e.g., PEGylated polypeptides)), antibodies and the like), antigenic substances (e.g., which may be a component of a vaccine), anti-blood clogging agents, compounds to treat neurogenerative diseases, anesthetic agents such as; benzocaine, chloroprocaine, cocaine, procaine, tetracaine, bupivacaine, lidocaine, mepivacaine, fentanyl and trimecaine, analgesic agents such as diclofenac and molecules to enable ion-gradient loading into the liposome such as ammonium sulfate, triethylamine sulfate, the triethylamine salt of sucrose octasulfate, triethylamine polyphosphate, ammonium salt of phytic acid, the sodium, triethylamine or ammonium salt of acetic acid, the sodium, triethylamine or ammonium salt of oxalic acid, the sodium, triethylamine or ammonium salt of propanic acid, the sodium, triethylamine or ammonium salt of succinic acid, the sodium, triethylamine or ammonium salt of 1, 2, 3, 4 butane tetracarboxylic acid, the sodium, triethylamine or ammonium salt of pyridine-2,3,5,6 tetracarboxylic acid, the sodium, triethylamine or ammonium salt of 1,2,4,5-benzenetetracarboxylic acid, the sodium, triethylamine or ammonium salt of 1,2,4,5-cyclohexanetetracarboxylic acid, the sodium, triethylamine or ammonium salt of 1,3,5-benzenetricarboxylic acid, the acetate salt of a polyamine such as spermine, spermidine, tris aminoethylamine and the like

Nutriceutical agents (e.g., flavonoids, antioxidants such as gamma-linolenic acid, beta carotene, anthocyanins, beta-sitosterol) and dietary supplements (e.g., vitamins) can also be used as payloads in the lipid compositions of the present disclosure.

Exemplary cosmeceutical agents that can serve as payloads of the compositions of the present disclosure can include hydrating agents, proteins (e.g., collagen), vitamins, phytochemicals, enzymes, antioxidants, essential oils, UV protective agents (e.g., oxybenzone), cleansing agents, dyes, fragrances, and the like (e.g., such as may find use cosmetics, toiletries, fragrances, perfumes, skin care products and beauty aids). Retinoic acid is of particular interest, particularly emulsions of sterol-modified amphiphilic lipids having retinoic acid for skin care products.

Diagnostic agents include detectable labels, which can be radiolabels, fluorophores, luminophores, nuclear magnetic resonance contrast agents such as gadolinum, positron emission tomography labels and the like. In some embodiments, the liposome itself can serve as a diagnostic agent, e.g., as in use as micro-bubbles in ultrasound diagnosis.

Of particular interest are sterol-modified amphiphilic lipid-containing compositions that serve as drug carriers for toxic drugs (such as various cancer chemotherapeutics, doxorubicin, danorubicin, camptothecin, and the like) and/or for drugs that have poor water solubility (e.g., amphotericin B, retinoic acid, and the like). Also of particular interest are sterol-modified amphiphilic lipid-containing compositions that serve as vaccine carriers, particularly those that exhibit storage stability (e.g., at ambient temperature and/or 4° C.).

Sterol-Modified Amphiphilic Lipids as Active Agents or Prodrugs

In some embodiments, the sterol-modified amphiphilic lipid or a component of the compound serves as the payload. For example, the sterol-modified amphiphilic lipid can itself be a drug or a prodrug. For example, the sterol of the sterol-modified amphiphilic lipid can be an agent that provides a beneficial effect when present in the sterol-modified amphiphilic lipid and/or when released from the sterol-modified amphiphilic lipid following cleavage of a cleavable linker. In one exemplary embodiment, the sterol of the sterol-modified amphiphilic lipid is β-sitosterol, which can find use in hypercholesterolemia therapy. Alternatively or in addition, the non-sterol hydrophobic tail of the sterol-modified amphiphilic lipid can provide be an agent that s provides a beneficial effect when present in the sterol-modified amphiphilic lipid and/or when released from the compound by cleavage of a cleavable linker. For example, the non-sterol hydrophobic tail of the sterol-modified amphiphilic lipid can be a retinoid acid.

Other Components

The sterol-modified amphiphilic lipid-containing compositions can include other active or inert agents in addition to the payload. For example, the liposome composition can include a drug-protective compound which can provide for reduced toxicity of a drug to be delivered using the composition and/or reduced toxicity of a component of the liposome. For example, such additional agents can include lipophilic free radical scavengers, such as alpha-tocopherol, or a pharmacologically acceptable analog or ester thereof, such as alpha-Tocopherol succinate. Other suitable free radical quenchers include butylated hydroxytoluene (BHT), propyl gallate (Augustin), and their pharmacologically acceptable salts. Additional lipophilic free radical quenchers which are acceptable for administration in humans may also be used. Such additional agents can be co-entrapped with the agent of interest by, for example, by encapsulation or membrane binding. The sterol-modified amphiphilic lipid-containing compositions can also include one or more imaging agents so the disposition of the drug-loaded liposome can be determined and/or followed in vivo.

Aqueous suspensions of liposomes of the present disclosure may advantageously include an agent to enhance resistance of the liposome to reduced oxidative degradation of liposome lipids. A water-soluble iron-specific chelator, such as desferal (ferrioxamine), is exemplary of such agents.

Formulations

Compositions can be formulated for a variety of different routes of administration including, parenteral, enteral, nasal, and pulmonary administration, and can include one or more excipients. Exemplary formulations include topical, transdermal, injectable (e.g., intravenous, intramuscular, subcutaneous), aerosol, and oral formulations, and can be formulated as pharmaceutical preparations, cosmeceutical preparations, nutriceutical preparations, and the like. Compositions containing sterol-modified amphiphilic lipids can be lyophilized, or may be stored in solution.

Accordingly, the sterol-modified amphiphilic lipid-containing compositions include formulations comprising a sterol-modified amphiphilic lipid and an acceptable carrier or vehicle. Acceptable dosage forms for the formulations include, but are not limited to, aqueous solutions, suspensions, dispersions, emollients, lotions, creams, salves, balms and ointments. The sterol-modified amphiphilic lipid compositions can also be administered in a solid form by way of a tablet or capsule, for example, to be dissolved in the digestive tract, as well as suppositories. The compositions can also be provided in a device such as patch, bandage, and the like.

Topical formulations, in addition to sterol-modified amphiphilic lipids and, where desired, a payload agent, can include a pharmaceutically acceptable topical carrier suitable for application to the skin or mucosa of an animal. Topical formulations can be provided in a variety of suitable dosage forms including but not necessarily limited to lotions, gels, salves, creams, balms, ointments and the like. These compositions may be in the form of aqueous solutions, or in the form of emulsions, e.g., oil and water emulsions (e.g., oil-in-water emulsions, or water-in-oil emulsions). Where desired, topical formulations may include penetration enhancers or other agents to aid in delivery through the skin.

The dosage forms contemplated herein can generally be formulated using physiologically acceptable carriers, excipients, stabilizers and the like, and may be provided in sustained release or timed release formulation. Acceptable carriers, excipients and diluents for therapeutic use are well known in the pharmaceutical field, and are described, for example, in Remington's Pharmaceutical Science (A. R. Gennaro Edit., Mack Publishing Co., 1985). Such materials are non-toxic to the recipients at the dosages and concentrations employed, and include buffers such as phosphate, citrate, acetate and other organic acid salts, antioxidants such as ascorbic acid, low molecular weight (less than about 10 residues) peptides such as polyarginine, proteins, such as serum albumin, gelatin and immunoglobulins, hydrophilic polymers such as poly(vinyl pyrrolidinone), amino acids such as glycine, glutamic acid, aspartic acid and arginine, monosaccharides, disaccharides, and other carbohydrates, including cellulose and its derivatives, glucose, mannose and dextrins, chelating agents such as EDTA, sugar alcohols such as mannitol and sorbitol, and in the topical formulations conventional cationic and nonionic surfactants such as TWEEN, PLURONICS, and PEG.

Dosage formulations to be used for therapeutic administration must be sterile. Sterility is readily accomplished by filtration through sterile membranes, or by other conventional methods such as irradiation or treatment with gases, heat, or high pressure. The pH of the dosage formulations of this invention typically will be between 3 and 11, and more preferably from 5 to 9. Subjects in need of treatment (typically mammalian) using the dosage formulations of this invention can be administered dosages that will provide optimal efficacy. The dose and method of administration will vary from subject to subject and be dependent upon such factors as the type of host being treated, and in the case of animals, its sex, weight, diet, concurrent medication, overall clinical condition, the particular hydrophobic compounds employed, the specific use for which these compounds are employed, and other factors which those skilled in the arts will recognize. The determination of effective dosage levels, that is, the dosage levels necessary to achieve the desired result, will be within the ambit of one skilled in the art.

The formulations can be prepared for storage under conditions suitable for the preservation of an activity of any payload, as well as for maintenance of the integrity of the sterol-modified amphiphilic lipid and other lipid that may be present. As illustrated in the Examples, liposomes containing sterol-modified amphiphilic lipid were stable at 4° C. for a year or more; thus storage at 4° C. is suitable for long-term maintenance of the sterol-modified amphiphilic lipid compositions described herein.

### Methods of Use of Sterol-Modified Amphiphilic Lipids

Sterol-modified amphiphilic lipid-containing compositions can be used in a variety of different pharmaceutical, cosmeceutical, diagnostic and biomedical applications. Exemplary uses are described below.

For example, pharmaceutical, nutriceutical and cosmeceutical applications generally involve administering a composition comprising a sterol-modified amphiphilic lipid to an animal subject. Administering is generally accomplished by contacting the animal with the composition, which can be by any suitable route (e.g., parenteral, enteral, nasal, pulmonary, etc.). In the context of these applications, the animal is a subject in need of treatment or for which treatment is desired as the subject is in need of treatment or may benefit from such treatment. “Animal subject” as used herein generally refers to a subject which, in the context of a therapeutic method, is in need to therapy and/or, in the context of a diagnostic method, is a subject suspected of having a condition that can be detected by the diagnostic method. Subjects include animals, including mammals such as humans, livestock, domestic pets, and the like.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The sterol-modified amphiphilic lipid-containing composition and its payload will be selected according to the subject and condition to be treated, as well as the benefit sought from administration.

Sterol-modified amphiphilic lipid-containing compositions also find use in diagnostics methods. Such methods include diagnosis of a condition by detection of an analyte present in a biological sample from an animal subject.

“Diagnosis” as used herein generally includes determination of a subject's susceptibility to a disease or disorder, determination as to whether a subject is presently affected by a disease or disorder, prognosis of a subject affected by a disease or disorder, and use of therametrics (e.g., monitoring a subject's condition to provide information as to the effect or efficacy of therapy). The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

In general, the diagnostic methods using sterol-modified amphiphilic lipid-containing compositions involves detecting the presence or absence of an analyte in fluid, the method comprising contacting the fluid with a sterol-modified amphiphilic lipid-containing composition, and detecting a change in the lipid composition, where the change in the lipid composition is indicative of the presence or absence of the analyte. The change in the lipid composition can be, for example, a change in color (e.g., due to a change in emission wavelength of an encapsulated fluorophore), due to an increase or decrease in polymerization of a polymerizable sterol-modified amphiphilic lipid present in the sterol-modified amphiphilic lipid-containing composition, a change in size or integrity of a sterol-modified amphiphilic lipid-containing liposome, and the like. As such the change in the lipid composition can be detected by evaluating a property of the lipid composition such as an optical property (e.g., reflectivity), phase transition, and the like.

Other Uses

The sterol-modified amphiphilic lipid-containing compositions also find a variety of uses in applications that are not associated directly with therapy or diagnosis. For example, sterol-modified amphiphilic lipid-containing compositions can be used to facilitate transfer of a payload (e.g., nucleic acid, polypeptide) to a cell in vitro. In this method, a cultured animal cell (e.g., a cultured mammalian cell, such as a human cell) is contacted with a sterol-modified amphiphilic lipid composition containing a payload of interest to facilitate delivery of the payload to the cell. Such methods can be used to accomplish introduction of nucleic acid, polypeptide, or other compound that does not readily cross an animal cell membranes.

In another example, sterol-modified amphiphilic lipids having sphingosine find use in modeling of lipid rafts, as well as in the delivery of membrane proteins.

Sterol-modified amphiphilic lipids can also be used as artificial bilayers, and thus can be used in settings such as biosensors. Biosensors that involve use of artificial bilayers are well known in the art. They are particularly suitable where a lipid surface is placed directly in contact with blood, plasma, serum or other body fluids containing cells, proteins or lipids.

### Kits and Systems

Kits and systems are provided which can facilitate the production and/or use of the compositions disclosed herein. Kits contemplated herein can include one or more of a sterol-modified amphiphilic lipid, an agent of interest for delivery, which may be provided in separate containers or, more usually, in a single composition in a sterile container.

In addition, the kit can contain instructions for using the components of the kit, particularly the compositions of the invention that are contained in the kit.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Methods and Materials

The following are general materials and protocols used in the Examples below.

Reagents: Glycerophosphocholine was obtained from SACHEM (Torrance, Calif.). Lyso-phospholipids were purchased from Avanti Polar Lipids (Alabaster, Ala.). Other reagents were from Aldrich (Milwaukee, Wis.). Solvents were used either directly or purified and dried before use according to the standard protocol.

Techniques. TLC analyses were performed on 0.25-mm silica gel F₂₅₄ plates using a variety of developing systems: (A) CHCl₃/MeOH/NH₄OH (65/25/4), (B) CHCl₃/MeOH/NH₄OH (65/35/5), (C)CHCl₃/MeOH/H₂O (65/25/4), (D) hexane/EtOAc (2/1), (E) hexane/EtOAc (10/1), (F) hexane/EtOAc (5/1), (G) toluene/ether (9/1), (H) toluene/ether (1/1). High performance flash chromatography (HPFC) was carried out on a Biotage (Charlottesville, Va.) Horizon™ HPFC™ system with pre-packed silica gel columns (60 Å, 40-63 μm). Unless noted otherwise, the ratios describing the composition of solvent mixtures represent relative volumes. ¹H NMR spectra were acquired on a Varian 400 MHz instrument or on a Bruker 300 mHz instrument. Chemical shifts are expressed as parts per million using tetramethylsilane as internal standard. J values are in Hertz. MALDI-TOF mass spectra were obtained at the Mass Spectrometry Facility, University of California San Francisco.

Synthetic methods. The general procedures used in the synthesis are described below.

Protection of 3-Hydroxy Group of 1-Substituted Glycerol: A mixture of 1-substituted glycerol and triphenyl chloride (1.5 equiv.) in anhydrous pyridine was stirred at 50° C. for 18 h under anhydrous condition. After cooling to room temperature (r.t., ca. 23° C.), the mixture was poured into ice-cold water, and extracted with 3 portions of hexane. Undissolved triphenylmethanol was removed by filtration. The filtrate was washed 3 times with water and dried over anhydrous sodium sulfate. The solvent was evaporated and the residue was dissolved in minimum amount of hexane. Additional triphenyl methanol was precipitated from the solution by standing overnight at 4° C. The solid was filtered off, and the filtrate was evaporated to dryness. The residue was dried over high vacuum and used directly for next step reaction. The reaction was monitored by TLC and yield was generally 80-90%.

Removal of Trityl Group from 1,2-Substituted-3-Trityl Glycerol: 1,2-substituted-3-trityl glycerol in chloroform was treated with boron trifluoride diethyl etherate (4 equiv.) at 0° C. for 3 h. The solution was washed with water/chloroform/methanol (2:2:1). The organic layer was dried over sodium sulfate and evaporated. The residue was dried and used directly for next step reaction. The reaction was monitored by TLC and yield was above 90%.

Phosphorylation of 1,2-Substituted-Glycerol: A solution of 1,2-substituted glycerol and anhydrous pyridine (2 equiv.) in anhydrous tetrahydrofuran (THF) was added dropwise to the freshly distilled phosphorus oxychloride (1.1 equiv.) in THF with stirring at 0° C. Stirring was continued for 2-3 h at 0° C. Then 10% sodium bicarbonate (ca. 5 equiv.) was added, and the mixture was stirred for 15 min at 0° C. The solution was then poured on ice water, acidified with HCl (pH ca. 2), and extracted with diethyl ether. The product in aqueous layer was precipitated by adding acetone into water. The precipitate was combined with product from the ether extract, azeotropically dried with toluene, and used directly for next step reaction. Yield was generally above 90%.

1,2-Substituted-Glycero-Phosphocholine: 1,2-Substituted-glycero phosphate, choline tetraphenyl borate (2 equiv.) and 2,4,6-triisoproylbenzene sulfonyl chloride (TPS) (2.5 equiv.) were dissolved in anhydrous pyridine with brief warming, then stirred for 1 h at 70° C. and 3 h at room temperature. After the addition of water, the solvents are removed by rotary evaporation. The residue was extracted with diethyl ether twice. The extract was combined and evaporated. The crude product was purified by HPFC. Yield of this step is generally 80-90%.

### Example 1

### Preparation of Lipid SML1a, SML1b, and SML1c

A synthetic scheme for the synthesis of lipids referred to herein as SML1a, SML1b, and SML1c (referred to collectively as SML1a-SML1c) is outlined in Scheme 1. This scheme is exemplified below by the detailed description of the synthesis of lipids SML1a-c.

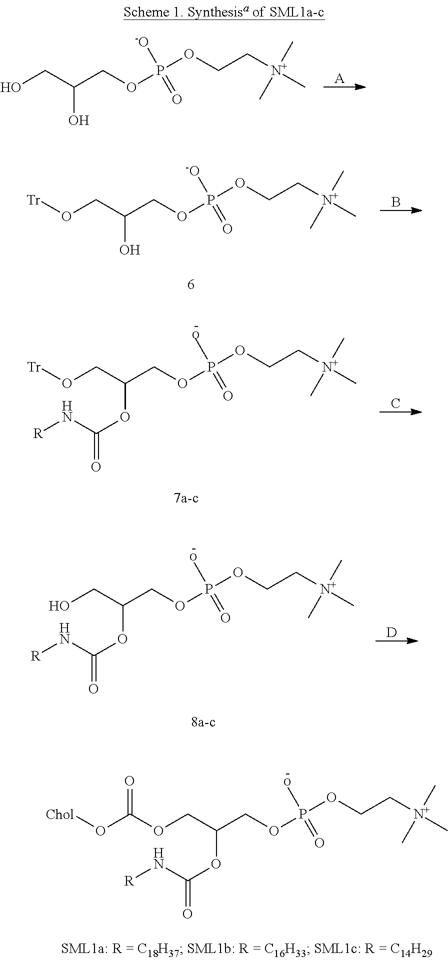

^{a}Reagents and conditions. (A) Trityl chloride (1.03 equiv.), ZnCl₂ (0.95 equiv.), DMF, 4° C., 10 h; (B) Alkyl isocyanate (1.0 equiv.), DMSO, 100° C., 24 h; (C) TFA in CHCl₃ (16.7%), r.t., 4 h; (D) cholesteryl chloroformate (5 equiv.), DIPEA (5.2 equiv.), CHCl₃, r.t., 16 h. “Tr” represents a trityl group. “Chol-OH”=cholesterol.

1-O-Trityl-sn-glycero-3-phosphocholine (6): Zinc chloride powder (anhydrous, 25g, 175 mmol) was added to the suspension of glycerophosphocholine (50 g, 185 mmol) in anhydrous DMF (500 mL). The mixture was stirred at r.t. for 30 min, and trityl chloride (53 g, 190 mmol) was added at 4° C. The reaction was kept at 4° C. for 10 h. Then, the crude product was precipitated by the addition of 1 L diethyl ether. The oily product was dissolved in 1 L chloroform/isobutanol (2:1), washed with 300 ml 4% aqueous ammonia, dried over anhydrous sodium sulfate. After the evaporation of the volatiles, the crude product was azeotropically dried with toluene. The residue was triturated with acetonitrile for 5 h at r.t. The white precipitate was then collected and dried over high vacuum. Yield: 45.2 g (49% wrt glycerophosphocholine). TLC: R_{f}=0.08 (eluent A). ¹H NMR (MeOH-d₄), δ3.12 (m, 2H); 3.15 (s, 9H); 3.56 (m, 2H); 3.90 (m, 2H); 4.01 (m, 1H); 4.20 (m, 2H); 7.27 (m, 9H); 7.42 (m, 6H). MALDI-MS calcd for C₂₇H₃₅NO₆P⁺ [M+H]⁺ 500.22. Found 500.31.

1-O-Trityl-2-stearylcarbamoyl-sn-glycero-3-phosphocholine (7a): To a solution of 6 (1 g, 2 mmol) in dimethylsulfoxide (anhydrous, 10 mL) was added octadecylisocyanate (0.6 g, 2 mmol). The reaction mixture was stirred at 100° C. under N₂ for 24 h. After the evaporation of the solvent, the residue was extracted with methanol. The white solid was filtered off and the filtrate was evaporated to dryness. The Crude product was purified by HPFC (CHCl₃/MeOH/H₂O, 35/13/2). Yield: 450 mg (28.3% wrt 6). TLC: R_{f}=0.25 (eluent A). ¹H NMR (CDCl₃), δ0.89 (t, J=6.4, 3H); 1.25-1.31 (m, 30H); 1.47 (m, 2H); 3.01 (m, 2H); 3.20 (s, 9H); 3.27 (m, 2H); 3.72 (m, 2H); 4.03-4.12 (m, 3H); 4.24 (m, 2H); 5.07 (br, 1H); 7.20 (m, 9H); 7.41 (m, 6H). MALDI-MS calcd for C₄₆H₇₂N₂O₇P⁺ [M+H]⁺ 795.51. Found 795.52.

1-O-Trityl-2-palmitylcarbamoyl-sn-glycero-3-phosphocholine (7b): This compound was synthesized according to the same procedure of 7a and used directly for next step reaction.

1-O-Trityl-2-myristylcarbamoyl-sn-glycero-3-phosphocholine (7c): This compound was synthesized according to the same procedure of 7a and used directly for next step reaction.

1-Hydroxy-2-stearylcarbamoyl-sn-glycero-3-phosphocholine (8a): Compound 7a (420 mg, 0.52 mmol) was treated with trifluoroacetic acid (TFA, 1 mL) in chloroform (5 mL) at r.t. for 4 h. The volatiles were evaporated and the residue was purified by HPFC (CHCl₃/MeOH/H₂O, 10/5/1). Yield: 320 mg (99% wrt 7a). TLC: R_{f}=0.05 (eluent B). ¹H NMR (CDCl₃), δ0.88 (t, J=6.4, 3H); 1.21-1.31 (br, 30H); 1.47 (m, 2H); 3.03 (m, 1H); 3.11 (m, 1H); 3.29 (s, 9H); 3.66 (m, 2H); 3.76 (m, 2H); 3.99 (m, 2H); 4.30 (m, 2H); 4.78 (m, 1H); 6.51 (br, 1H). MALDI-MS calcd for C₂₇H₅₈N₂O₇P⁺ [M+H]⁺ 553.40. Found 553.38.

1-Hydroxy-2-palmitylcarbamoyl-sn-glycero-3-phosphocholine (8b): This compound was synthesized according to the same procedure of 8a. TLC: R_{f}=0.05 (eluent B). ¹H NMR (CDCl₃), δ0.89 (t, J=6.4, 3H); 1.21-1.31 (br, 26H); 1.48 (m, 2H); 3.03 (m, 1H); 3.11 (m, 1H); 3.30 (s, 9H); 3.66-3.76 (m, 4H); 4.0 (m, 2H); 4.30 (m, 2H); 4.79 (m, 1H); 6.52 (br, 1H). MALDI-MS calcd for C₂₅H₅₄N₂O₇P⁺ [M+H]⁺ 525.37. Found 525.28.

1-Hydroxy-2-myristylcarbamoyl-sn-glycero-3-phosphocholine (8c): This compound was synthesized according to the same procedure of 8a. TLC: R_{f}=0.05 (eluent B). ¹H NMR (CDCl₃), δ0.89 (t, J=6.4, 3H); 1.21-1.31 (m, 22H); 1.47 (m, 2H); 3.03 (m, 1H); 3.11 (m, 1H); 3.29 (s, 9H); 3.66-3.76 (m, 4H); 3.99 (m, 2H); 4.30 (m, 2H); 4.78 (m, 1H); 6.52 (br, 1H). MALDI-MS calcd for C₂₃H₅₀N₂O₇P⁺ [M+H]⁺ 497.34. Found 497.36.

1-Cholesterylcarbonoyl-2-stearylcarbamoyl-sn-glycero-3-phosphocholine (SML1a, Ch_{c}SₐPC): To a solution of solution of 8a (0.3 g, 0.54 mmol) and diisopropylethylamine (DIPEA, 0.5 mL, 2.8 mmol) in dry ethanol-free chloroform (10 mL), was added dropwise the solution of cholesteryl chloroformate (1.21 g, 2.7 mmol) in ethanol-free chloroform (5 mL) at r.t. After 16 h reaction at r.t., volatiles were evaporated and the residue was purified by HPFC(CHCl₃/MeOH/H₂O, 40/18/3). Yield: 438 mg (84% wrt 8a). TLC: R_{f}=0.28 (eluent A). ¹H NMR (CDCl₃), δ0.69 (s, 3H); 0.85-1.65 (m, 68H); 1.78-2.01 (m, 5H); 2.38 (m, 2H); 3.03 (m, 1H); 3.17 (m, 1H); 3.38 (s, 9H); 3.88 (m, 2H); 4.0 (m, 2H); 4.25 (m, 1H); 4.36 (m, 4H); 5.08 (m, 1H); 5.39 (1H, d, J=4.4); 6.02 (br, 1H). MALDI-MS calcd for C₅₅H₁₀₂N₂O₉P⁺ [M+H]⁺ 965.73. Found 965.68.

1-Cholesterycarbonoyl-2-palmitylcarbamoyl-sn-glycero-3-phosphocholine (SML1b, Ch_{c}PₐPC): This compound was synthesized according to the same procedure of SML1a. TLC: R_{f}=0.23 (eluent NMR (CDCl₃), δ0.69 (s, 3H); 0.85-1.65 (m, 64H); 1.78-2.01 (m, 5H); 2.37 (m, 2H); 3.0 (m, 1H); 3.16 (m, 1H); 3.37 (s, 9H); 3.88 (m, 2H); 3.98 (m, 2H); 4.24 (m, 1H); 4.36 (m, 4H); 5.07 (m, 1H); 5.39 (1H, d, J=4.4); 6.17 (br, 1H). MALDI-MS calcd for C₅₃H₉₈N₂O₉P⁺ [M+H]⁺ 937.70. Found 937.69.

1-Cholesterylcarbonoyl-2-myristylcarbamoyl-sn-glycero-3-phosphocholine (SML1c, Ch_{c}MₐPC): This compound was synthesized according to the same procedure of SML1a. TLC: R_{f}=0.25 (eluent A). ¹H NMR (CDCl₃), δ0.69 (s, 3H); 0.85-1.65 (m, 60H); 1.78-2.06 (m, 5H); 2.38 (m, 2H); 3.03 (m, 1H); 3.17 (m, 1H); 3.38 (s, 9H); 3.88 (m, 2H); 4.0 (m, 2H); 4.25 (m, 1H); 4.36 (m, 4H); 5.08 (m, 1H); 5.40 (1H, d, J=4.4); 6.02 (br, 1H). MALDI-MS calcd for C₅₁H₉₄N₂O₉P⁺ [M+H]⁺ 909.67. Found 909.70.

### Example 2

### Preparation of Lipid SML2a, SML2b, SML2c, and SML2d

A synthetic scheme for the synthesis of lipids SML2a, SML2b, SML2c, and SML2d (referred to collectively as SML2a-SML2d) is outlined in Scheme 2. This scheme is exemplified below by the detailed description of the synthesis of lipids SML2a-d.

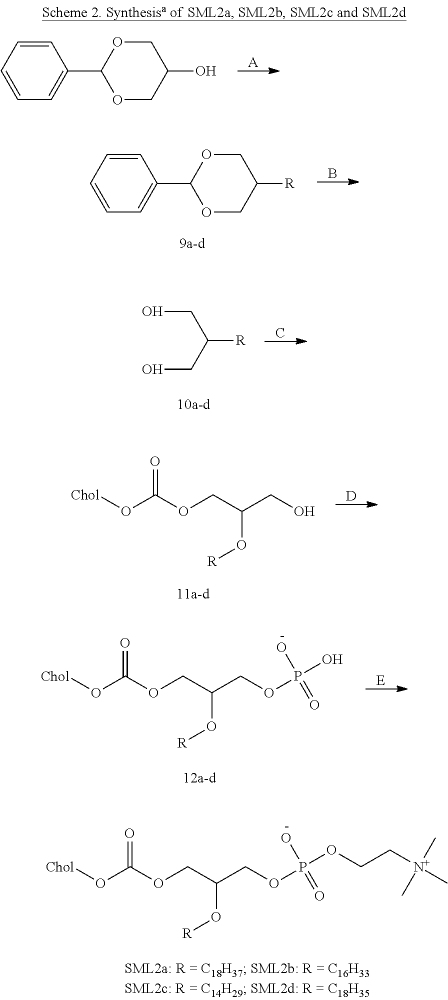

^{a}Reagents and conditions. (A) 1) NaH (1.2 equiv.), toluene, r.t., 30 min; 2) iodoalkane (1.25 equiv.), reflux, overnight; (B) HCl (conc.) in MeOH (10%), reflux, 5 h; (C) Cholesteryl chloroformate (1.05 equiv.), DIPEA (1.4 equiv.), DMAP (0.5 equiv.), CHCl₃, 0° C., 0.5 h then r.t., overnight; (D) POCl₃ (1.1 equiv.), pyridine (2 equiv.), THF, 0° C., 2-3 h; (E)

Choline tetraphenyl borate (2 equiv.), TPS (2.5 equiv.), pyridine, 70° C., 1 h, then r.t., 3 h. “Chol-OH”=cholesterol.

1,3-Benzylidene-2-stearyl-glycerol (9a): A solution of 1,3-benzylidene glycerol (7.2 g, 40 mmol) in toluene (100 mL) was added to NaH (60% in mineral oil, 1.92 g, 48 mmol, washed with hexane) suspension in toluene (30 mL) at r.t. with stirring. Then 1-iodo-octadecane (20 g, 50 mmol) in toluene (40 mL) was added dropwise into the reaction mixture. After the addition, the mixture was refluxed under nitrogen overnight, and cooled to r.t. Excessive NaH was destroyed by careful addition of water into the mixture. The reaction mixture was then washed with water (100 mL×2). The organic layer was collected, dried over sodium sulfate. Solvent was evaporated and the residue was used directly for next step reaction.

1,3-Benzylidene-2-palmityl-glycerol (9b): This compound was synthesized according to the same procedure of 9a.

1,3-Benzylidene-2-myristyl-glycerol (9c): This compound was synthesized according to the same procedure of 9a.

1,3-Benzylidene-2-oleyl-glycerol (9d): This compound was synthesized according to the same procedure of 9a.

2-Stearyl-glycerol (10a): The crude product of 9a was hydrolyzed by refluxing in the mixed solution of HCl (conc., 30 mL) and methanol (270 mL) for 5 h. The reaction mixture was cooled to r.t. and evaporated under reduced pressure. The residue was dissolved in diethyl ether (300 mL) and washed consecutively with sodium hydroxide solution (0.5 M, 100 mL) and water (150 mL×2). The ether layer was then dried and evaporated. The crude product was purified by HPFC (30-80% ethyl acetate in hexane). Yield: 11.3 g (82% wrt 1,3-benzylidene glycerol). TLC: R₁=0.17 (eluent D). ¹H NMR (CDCl₃), δ0.86 (t, J=6.4, 3H); 1.29 (br, 30H); 1.58 (m, 2H); 3.44-3.78 (m, 7H). MALDI-MS calcd for C₂₁H₄₅O₃⁺ [M+H]⁺ 345.34. Found 345.33.

2-Palmityl-glycerol (10b): This compound was synthesized according to the same procedure of 10a. TLC: R_{f}=0.16 (eluent D). ¹H NMR (CDCl₃), δ0.86 (t, J=6.4, 3H); 1.29 (br, 26H); 1.57 (m, 2H); 3.44-3.78 (m, 7H). MALDI-MS calcd for C₁₉H₄₁O₃⁺ [M+H]⁺ 317.31. Found 317.28.

2-Myristyl-glycerol (10c): This compound was synthesized according to the same procedure of 10a. TLC: R_{f}=0.18 (eluent D). ¹H NMR (CDCl₃), δ0.87 (t, J=6.4, 3H); 1.29 (br, 22H); 1.58 (m, 2H); 3.44-3.78 (m, 7H). MALDI-MS calcd for C₁₇H₃₇O₃⁺ [M+H]⁺ 289.28. Found 289.26

2-Oleyl-glycerol (10d): This compound was synthesized according to the same procedure of 10a. TLC: R_{f}=0.17 (eluent D). ¹H NMR (CDCl₃), δ0.87 (t, J=6.4, 3H); 1.29 (br, 22H); 1.58 (m, 2H); 2.0 (m, 4H); 3.44-3.78 (m, 7H); 5.34 (m, 2H). MALDI-MS calcd for C₂₁H₄₃O₃⁺ [M+H]⁺. 343.32. Found 343.32.

1-Cholesterylcarbonoyl-2-stearyl-glycerol (11a): To a solution of 10a (0.7 g, 2 mmol), DIPEA (0.5 mL, 2.8 mmol) and DMAP (0.12 g, 1 mmol) in dry ethanol-free chloroform (10 mL), was added dropwise cholesteryl chloroformate (0.94 g, 2.1 mmol) chloroform solution (10 mL) at 0° C. The reaction mixture was stirred at 0° C. for 0.5 h, then at r.t. overnight. The volatiles were evaporated, and the crude product was purified by HPFC (5-15% ethyl acetate in hexane). TLC: R_{f}=0.41 (eluent E). ¹H NMR (CDCl₃), δ0.69 (s, 3H); 0.85-1.65 (m, 68H); 1.78-2.01 (m, 5H); 2.40 (m, 2H); 3.52 (m, 2H); 3.60 (m, 2H); 3.68 (m, 1H); 4.22 (m, 2H); 4.43 (m, 1H); 5.40 (1H, d, J=4.4); MALDI-MS calcd for C₄₉H₈₉O₅⁺ [M+H]⁺ 757.67. Found 757.68.

1-Cholesterycarbonoyl-2-palmityl-glycerol (11b): This compound was synthesized according to the same procedure of 11a. TLC: R_{f}=0.39 (eluent E). ¹H NMR (CDCl₃), δ0.69 (s, 3H); 0.85-1.65 (m, 64H); 1.79-2.01 (m, 5H); 2.39 (m, 2H); 2.80 (m, 1H); 3.51 (m, 2H); 3.61 (m, 2H); 3.72 (m, 1H); 4.21 (m, 2H); 5.39 (1H, d, J=4.4); MALDI-MS calcd for C₄₇H₈₅O₅⁺ [M+H]⁺ 729.64. Found 729.61.

1-Cholesterycarbonoyl-2-myristyl-glycerol (11c): This compound was synthesized according to the same procedure of 11a. TLC: R_{f}=0.40 (eluent E). ¹H NMR (CDCl₃), δ0.69 (s, 3H); 0.85-1.65 (m, 60H); 1.78-2.02 (m, 5H); 2.38 (m, 2H); 2.82 (m, 1H); 3.51 (m, 2H); 3.60 (m, 2H); 3.72 (m, 1H); 4.22 (m, 2H); 5.41 (1H, d, J=4.4); MALDI-MS calcd for C₄₅H₈₁O₅⁺ [M+H]⁺ 711.69. Found 711.68.

1-Cholesterylcarbonoyl-2-oleyl-glycerol (11d): This compound was synthesized according to the same procedure of 11a. TLC: R_{f}=0.40 (eluent E). ¹H NMR (CDCl₃), δ0.69 (s, 3H); 0.85-1.65 (m, 60H); 1.78-2.01 (m, 9H); 2.38 (m, 2H); 3.51 (m, 2H); 3.60 (m, 2H); 3.71 (m, 1H); 4.22 (m, 2H); 4.46 (m, 1H); 5.35 (m, 2H); 5.40 (d, J=4.4, 1H); MALDI-MS calcd for C₄₉H₈₇O₅⁺ [M+H]⁺ 755.66. Found 755.62.

1-Cholesterylcarbonoyl-2-stearyl-rac-glycero-3-phosphate (12a): This compound was synthesized according to the general procedure of phosphorylation. TLC: R_{f}=0.05 (eluent A). MALDI-MS calcd for C₄₉H₈₉NaO₈P⁺ [M+Na]⁺ 859.62. Found 859.60.

1-Cholesterylcarbonoyl-2-palmityl-rac-glycero-3-phosphate (12b): This compound was synthesized according to the general procedure of phosphorylation. TLC: R_{f}=0.05 (eluent A). MALDI-MS calcd for C₄₇H₈₅NaO₈P⁺ [M+Na]⁺ 831.59. Found 831.61.

1-Cholesterylcarbonoyl-2-myristyl-rac-glycero-3-phosphate (12c): This compound was synthesized according to the general procedure of phosphorylation. TLC: R_{f}=0.05 (eluent A). MALDI-MS calcd for C₄₅H₈₁NaO₈P⁺ [M+Na]⁺ 803.56. Found 803.55.

1-Cholesterylcarbonoyl-2-oleyl-rac-glycero-3-phosphate (12d): This compound was synthesized according to the general procedure of phosphorylation. TLC: R_{f}=0.05 (eluent A). MALDI-MS calcd for C₄₉H₈₇NaO₈P⁺ [M+Na]⁺ 857.60. Found 857.62.

1-Cholesterylcarbonoyl-2-stearyl-rac-glycero-3-phosphocholine (SML2a, Ch_{c}SₑPC): This compound was synthesized according to the general procedure of phosphocholine synthesis. TLC: R_{f}=0.3 (eluent A). δ 0.69 (s, 3H); 0.85-1.65 (m, 68H); 1.78-2.04 (m, 5H); 2.38 (m, 2H); 3.41 (s, 9H); 3.52 (m, 2H); 3.68 (m, 1H); 3.88 (m, 4H); 4.19 (m, 1H); 4.35 (m, 4H); 5.39 (1H, d, J=4.4); MALDI-MS calcd for C₅₄H₁₀₁NO₈P⁺ [M+H]⁺ 922.73. Found 922.74.

1-Cholesterylcarbonoyl-2-palmityl-rac-glycero-3-phosphocholine (SML2b, Ch_{c}PₑPC): This compound was synthesized according to the general procedure of phosphocholine synthesis. TLC: R_{f}=0.3 (eluent A). δ0.69 (s, 3H); 0.85-1.65 (m, 64H); 1.78-2.04 (m, 5H); 2.38 (m, 2H); 3.41 (s, 9H); 3.52 (m, 2H); 3.68 (m, 1H); 3.88 (m, 4H); 4.19 (m, 1H); 4.35 (m, 4H); 5.39 (1H, d, J=4.4); MALDI-MS calcd for C₅₂H₉₇NO₈P⁺ [M+H]⁺ 894.69. Found 894.70.

1-Cholesterylcarbonoyl-2-myristyl-rac-glycero-3-phosphocholine (SML2c, Ch_{c}MₑPC): This compound was synthesized according to the general procedure of phosphocholine synthesis. TLC: R_{f}=0.3 (eluent A). δ0.69 (s, 3H); 0.85-1.65 (m, 60H); 1.78-2.04 (m, 5H); 2.38 (m, 2H); 3.41 (s, 9H); 3.52 (m, 2H); 3.68 (m, 1H); 3.88 (m, 4H); 4.19 (m, 1H); 4.35 (m, 4H); 5.39 (1H, d, J=4.4); MALDI-MS calcd for C₅₂H₉₇NO₈P⁺ [M+H]⁺ 866.66. Found 866.67.

1-Cholesterylcarbonoyl-2-oleyl-rac-glycero-3-phosphocholine (SML2d, Ch_{c}OₑPC): This compound was synthesized according to the general procedure of phosphocholine synthesis. TLC: R_{f}=0.3 (eluent A). δ0.69 (s, 3H); 0.85-1.65 (m, 60H); 1.78-2.04 (m, 9H); 2.39 (m, 2H); 3.40 (s, 9H); 3.54 (m, 2H); 3.70 (m, 1H); 3.84-3.97 (m, 4H); 4.20 (m, 1H); 4.35-4.44 (m, 4H); 5.35 (m, 2H); 5.39 (1H, d, J=4.4); MALDI-MS calcd for C₅₄H₉₉NO₈P⁺ [M+H]⁺ 920.71. Found 920.73.

### Example 3

### Preparation of Lipids SML3a, SML3b, SML3c, and SML3d

A synthetic scheme for the synthesis of lipid SML3a, SML3b, SML3c, and SML3d (referred to collectively as SML3a-SML3d) is outlined in Scheme 3. This scheme is exemplified below by the detailed description of the synthesis of lipids SML3a-d.

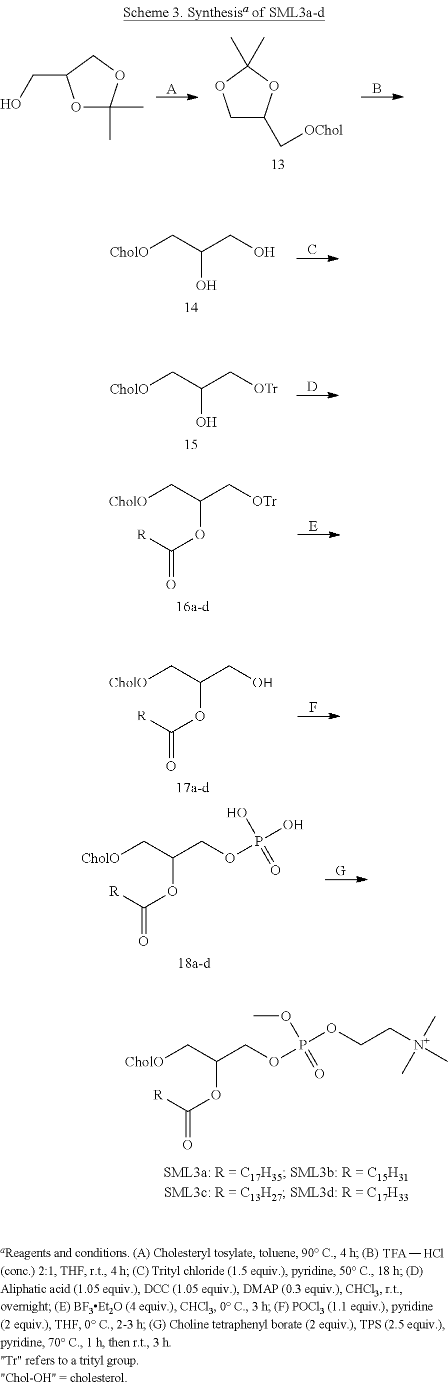

3-(2,3-Isopropylidene-1-glyceryl) cholesterol (13): A mixture of cholesteryl tosylate (50 g, 90 mmol) and solketal (250 mL, 2 mol) in toluene (50 mL) was stirred at 80-90° C. for 4 h under nitrogen. After cooling to r.t., toluene (300 mL) was added to the mixture. The mixture was washed with brine (300 mL). After separation, additional 200 mL toluene was added to the organic layer. The organic layer was then washed with brine (300 mL), dried, and evaporated to dryness. The crude product was used directly for next step reaction. TLC: R_{f}=0.55 (eluent G).

1-Glyceryl cholesterol (14): The crude product of 13 was dissolved in the mixed solvents of THF (130 mL)-TFA (40 mL)-HCl (conc., 20 mL). The mixture was kept at r.t. for 4 h. The volatiles were evaporated under reduced pressure. The residue was dissolved in CHCl₃/MeOH (400 mL/100 mL), and washed with water (100 mL). The organic layer was then dried over sodium sulfate, filtered, and evaporated. The crude product was purified by recrystallization from ethanol at −20° C. TLC: R_{f}=0.12 (eluent H), ¹H NMR (CDCl₃), δ0.69 (s, 3H); 0.85-1.65 (m, 33H); 1.78-2.31 (m, 7H); 3.10 (m, 1H); 3.45-3.70 (m, 4H); 3.79 (m, 1H); 5.34 (1H, d, J=4.4); MALDI-MS calcd for C₃₀H₅₃O₃⁺ [M+H]⁺ 461.40. Found 461.44.

1-Cholesteryl-3-trityl glycerol (15): The protection of 3-hydroxy group of 14 with trityl group was carried out according to the general procedure. Product was purified by HPFC (9%-25% ethyl acetate in hexane). TLC: R_{f}=0.38 (eluent F). ¹H NMR (CDCl₃), δ0.69 (s, 3H); 0.85-1.65 (m, 33H); 1.86 (m, 3H); 2.0 (m, 2H); 2.18 (m, 1H); 2.32 (m, 1H); 2.42 (br, 1H); 3.11-3.22 (m, 3H); 3.57 (m, 2H); 3.93 (m, 1H); 5.35 (1H, d, J=4.4); 7.28 (m, 9H); 7.43 (m, 6H). MALDI-MS calcd for C₄₉H₆₇O₃⁺ [M+H]⁺ 703.51. Found 703.53.

1-Cholesteryl-2-stearoyl-3-trityl glycerol (16a): To a solution of 15 (2.11 g, 3 mmol), stearic acid (0.94 g, 3.15 mmol), and 4-dimethylaminopyridine (DMAP, 0.13 g) in dry ethanol-free chloroform (20 mL), was added DCC (0.65 g, 3.15 mmol) at 0° C. The reaction mixture was stirred at 0° C. for 30 min, then r.t. overnight. White precipitate was filtered off, and the filtrate was evaporated to dryness. The crude product was purified by HPFC (1%-10% ethyl acetate in hexane). TLC: R_{f}=0.5 (eluent E). ¹H NMR (CDCl₃), δ0.69 (s, 3H); 0.85-1.65 (m, 66H); 1.81 (m, 3H); 2.0 (m, 2H); 2.19 (m, 1H); 2.26 (m, 1H); 2.35 (t, J=7.2, 2H); 3.12 (m, 1H); 3.25 (m, 2H); 3.67 (m, 2H); 5.17 (m, 1H); 5.32 (d, J=4.4, 1H); 7.27 (m, 9H); 7.44 (m, 6H). MALDI-MS calcd for C₆₇H₁₀₁O₄⁺ [M+H]⁺ 969.77. Found 969.73.

1-Cholesteryl-2-palmitoyl-3-trityl glycerol (16b): This compound was synthesized according to the same procedure of 16a. TLC: TLC: R_{f}=0.5 (eluent E). ¹H NMR (CDCl₃), δ 0.68 (s, 3H); 0.85-1.65 (m, 62H); 1.80 (m, 3H); 1.99 (m, 2H); 2.10 (m, 1H); 2.27 (m, 1H); 2.35 (t, J=7.2, 2H); 3.12 (m, 1H); 3.25 (m, 2H); 3.66 (m, 2H); 5.16 (m, 1H); 5.32 (d, J=4.4, 1H); 7.26 (m, 9H); 7.42 (m, 6H). MALDI-MS calcd for C₆₅H₉₇O₄⁺ [M H]⁺ 941.74. Found 941.72.

1-Cholesteryl-2-myristoyl-3-trityl glycerol (16c): This compound was synthesized according to the same procedure of 16a. TLC: R_{f}=0.5 (eluent E). ¹H NMR (CDCl₃), δ0.69 (s, 3H); 0.85-1.65 (m, 58H); 1.81 (m, 3H); 2.0 (m, 2H); 2.19 (m, 1H); 2.26 (m, 1H); 2.35 (t, J=7.2, 2H); 3.12 (m, 1H); 3.25 (m, 2H); 3.66 (m, 2H); 5.17 (m, 1H); 5.32 (d, J=4.4, 1H); 7.27 (m, 9H); 7.44 (m, 6H). MALDI-MS calcd for C₆₃H₉₃O₄⁺ [M+H]⁺ 913.71. Found 913.71.

1-Cholesteryl-2-oleoyl-3-trityl glycerol (16d): This compound was synthesized according to the same procedure of 16a. TLC: R_{f}=0.5 (eluent E). ¹H NMR (CDCl₃), δ0.69 (s, 3H); 0.85-1.65 (m, 58H); 1.81 (m, 3H); 2.01 (m, 6H); 2.11 (m, 1H); 2.27 (m, 1H); 2.37 (t, J=7.2, 2H); 3.13 (m, 1H); 3.24 (m, 2H); 3.66 (m, 2H); 5.17 (m, 1H); 5.35 (m, 3H); 7.28 (m, 9H); 7.45 (m, 6H). MALDI-MS calcd for C₆₇H₉₉O₄⁺ [M+H]⁺ 967.75. Found 967.74.

1-Cholesteryl-2-stearoyl glycerol (17a): The removal of trityl group was carried out according to the general procedure. The crude product was used directly for next step reaction. TLC: R_{f}=0.08 (eluent E).

1-Cholesteryl-2-palmitoyl glycerol (17b): The removal of trityl group was carried out according to the general procedure. The crude product was used directly for next step reaction. TLC: R_{f}=0.08 (eluent E).

1-Cholesteryl-2-myristoyl glycerol (17c): The removal of trityl group was carried out according to the general procedure. The crude product was used directly for next step reaction. TLC: R_{f}=0.08 in hexane/EtOAc (10/1).

1-Cholesteryl-2-oleoyl glycerol (17d): The removal of trityl group was carried out according to the general procedure. The crude product was used directly for next step reaction. TLC: R_{f}=0.08 (eluent E).

1-Cholesteryl-2-stearoyl-rac-glycero-3-phosphate (18a): This compound was synthesized according to the general procedure of phosphorylation. TLC: R_{f}=0.05 (eluent A).

1-Cholesteryl-2-palmitoyl-rac-glycero-3-phosphate (18b): This compound was synthesized according to the general procedure of phosphorylation. TLC: R_{f}=0.05 (eluent A).

1-Cholesteryl-2-myristoyl-rac-glycero-3-phosphate (18c): This compound was synthesized according to the general procedure of phosphorylation. TLC: R_{f}=0.05 (eluent A).

1-Cholesteryl-2-oleoyl-rac-glycero-3-phosphate (18d): This compound was synthesized according to the general procedure of phosphorylation. TLC: R_{f}=0.05 (eluent A).

1-Cholesteryl-2-stearoyl-rac-glycero-3-phosphocholine (SML3a, ChₑSPC): This compound was synthesized according to the general procedure of phosphocholine synthesis. TLC: R_{f}=0.31 (eluent A). ¹H NMR (CDCl₃), δ0.68 (s, 3H); 0.85-1.65 (m, 66H); 1.84 (m, 3H); 2.0 (m, 2H); 2.12 (m, 1H); 2.30 (m, 3H); 3.15 (m, 1H); 3.39 (s, 9H); 3.63 (m, 2H); 3.81 (m, 2H); 4.25-4.45 (m, 5H); 5.33 (d, J=4.4, 1H). MALDI-MS calcd for C₅₃H₉₉NO₇P⁺ [M+H]⁺ 892.72. Found 892.73.

1-Cholesteryl-2-palmitoyl-rac-glycero-3-phosphocholine (SML3b, ChₑPPC): This compound was synthesized according to the general procedure of phosphocholine synthesis. TLC: R_{f}=0.31 (eluent A). ¹H NMR (CDCl₃), δ0.68 (s, 3H); 0.85-1.65 (m, 62H); 1.83 (m, 3H); 1.99 (m, 2H); 2.11 (m, 1H); 2.29 (m, 3H); 3.14 (m, 1H); 3.38 (s, 9H); 3.64 (m, 2H); 3.82 (m, 2H); 4.24-4.44 (m, 5H); 5.32 (d, J=4.4, 1H). MALDI-MS calcd for C₅₁H₉₅NO₇P⁺ [M+H]⁺ 864.68. Found 864.70.

1-Cholesteryl-2-myristoyl-rac-glycero-3-phosphocholine (SML3c, ChₑMPC): This compound was synthesized according to the general procedure of phosphocholine synthesis. TLC: R_{f}=0.31 (eluent A). ¹H NMR (CDCl₃), δ0.68 (s, 3H); 0.85-1.65 (m, 58H); 1.83 (m, 3H); 1.99 (m, 2H); 2.12 (m, 1H); 2.30 (m, 3H); 3.14 (m, 1H); 3.39 (s, 9H); 3.62 (m, 2H); 3.83 (m, 2H); 4.22-4.42 (m, 5H); 5.33 (d, J=4.4, 1H). MALDI-MS calcd for C₄₉H₉₁NO₇P⁺ [M+H]⁺ 836.65. Found 836.65.

1-Cholesteryl-2-oleoyl-rac-glycero-3-phosphocholine (SML3d, ChₑOPC): This compound was synthesized according to the general procedure of phosphocholine synthesis. TLC: R_{f}=0.30 (eluent A). ¹H NMR (CDCl₃), δ0.68 (s, 3H); 0.85-1.65 (m, 58H); 1.83 (m, 3H); 2.0 (m, 6H); 2.12 (m, 1H); 2.29 (m, 3H); 3.13 (m, 1H); 3.36 (s, 9H); 3.60 (m, 2H); 3.82 (m, 2H); 4.25-4.45 (m, 5H); 5.32 (m, 3H). MALDI-MS calcd for C₅₃H₉₇NO₇P⁺ [M+H]⁺ 890.70. Found 890.67.

### Example 4

### Preparation of Lipids SML4a-4d

A synthetic scheme for the synthesis of lipids SML4a, SML4b, SML4c, and SML4d (referred to collectively as SML4a-d) is outlined in Scheme 4. This scheme is exemplified below by the detailed description of the synthesis of lipids SML4a-d.

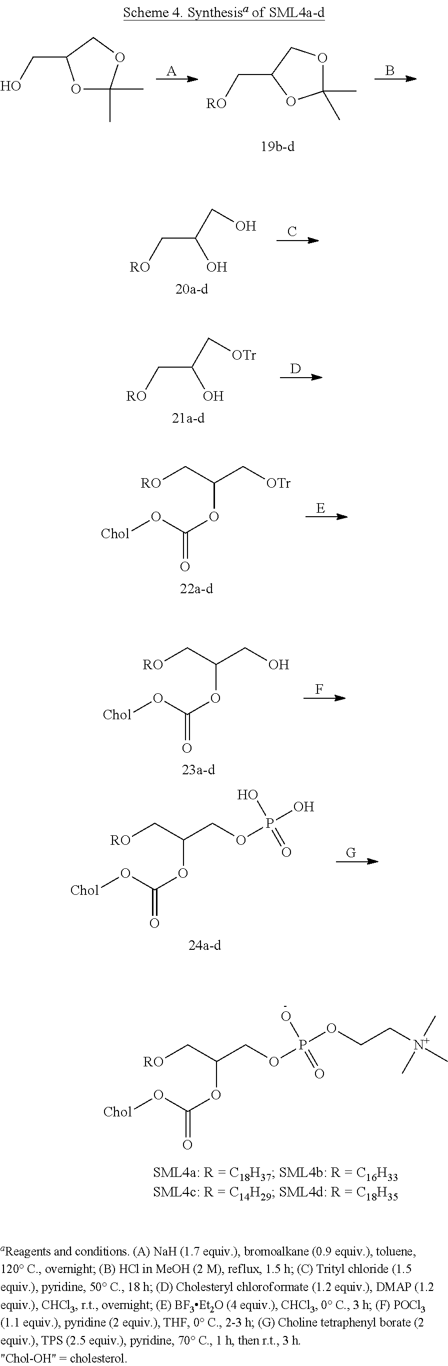

1-Palmityl-2,3-isopropylidene glycerol (19b): To a suspension of NaH (9 g, 0.225 mol) in 50 mL anhydrous toluene, was added solketal (19.8 g, 0.15 mol) dropwise. After the addition, the reaction mixture was stirred at 120° C. for 15 min. Then 1-bromotetradecane (40 mL, 0.134 mol) was added to the mixture and the reaction was kept at 120° C. overnight. After the reaction mixture was cooled to r.t., water (400 mL) was carefully added to destroy the excessive NaH, followed by the addition of hexane (400 mL). The organic layer was then washed with water (200 mL×2), dried over sodium sulfate, filtered, and evaporated. The residue was used directly for next step reaction.

1-Myristyl-2,3-isopropylidene glycerol (19c): This compound was synthesized according to the same procedure of 19b.

1-Oleyl-2,3-isopropylidene glycerol (19d): This compound was synthesized according to the same procedure of 19b.

1-Palmityl glycerol (20b): The crude product 19b was dissolved in 200 mL methanol and 40 mL conc. HCl and refluxed for 1.5 h. Then the mixture was cooled to r.t. and placed at 4° C. overnight. The crystal was collected and recrystallized from methanol. TLC: R_{f}=0.1 (eluent D). ¹H NMR (CDCl₃), δ0.87 (t, J=7.2, 3H); 1.27 (br, 26H); 1.53 (m, 2H); 2.52 (br, 2H); 3.30-3.90 (m, 7H). MALDI-MS calcd for C₁₉H₄₁O₃⁺ [M+H]⁺ 317.31. Found 317.30.

1-Myristyl glycerol (20c): This compound was synthesized according to the same procedure of 20b. TLC: R_{f}=0.1 (eluent D). ¹H NMR (CDCl₃), δ0.87 (t, J=7.2, 3H); 1.27 (br, 22H); 1.52 (m, 2H); 2.50 (br, 2H); 3.31-3.90 (m, 7H). MALDI-MS calcd for C₁₇H₃₇O₃⁺[M+H]⁺ 289.28. Found 289.25.

1-Oleyl glycerol (20d): This compound was synthesized according to the same procedure of 20b, but purified by HPFC (30-70% ethyl acetate in hexane). TLC: R_{f}=0.1 (eluent D). ¹H NMR (CDCl₃), δ0.87 (t, J=7.2, 3H); 1.27 (br, 22H); 1.57 (m, 2H); 2.0 (m, 4H); 2.62 (br, 2H); 3.36-3.54 (m, 4H); 3.68 (m, 2H); 3.85 (m, 1H); 5.34 (m, 2H). MALDI-MS calcd for C₂₁H₄₃O₃⁺ [M+H]⁺ 343.32. Found 343.36.

1-Stearyl-3-trityl glycerol (21a): The 3-trityl group was introduced according to the general procedure. TLC: R_{f}=0.11 (eluent E). ¹H NMR (CDCl₃), δ0.87 (t, J=7.2, 3H); 1.27 (br, 30H); 1.55 (m, 2H); 2.40 (br, 1H); 3.18 (m, 2H); 3.32-3.53 (m, 4H). 3.94 (m, 1H); 7.23 (m, 9H); 7.43 (m, 6H). MALDI-MS calcd for C₄₀H₅₉O₃⁺ [M+H]⁺ 587.45. Found 587.44.

1-Palmityl-3-trityl glycerol (21b): The 3-trityl group was introduced according to the general procedure. TLC: R_{f}=0.11 (eluent E). ¹H NMR (CDCl₃), δ0.87 (t, J=7.2, 3H); 1.27 (br, 26H); 1.55 (m, 2H); 2.42 (br, 1H); 3.18 (m, 2H); 3.32-3.53 (m, 4H). 3.94 (m, 1H); 7.22 (m, 9H); 7.42 (m, 6H). MALDI-MS calcd for C₃₈H₅₅O₃⁺ [M+H]⁺ 559.42. Found 559.40.

1-Myristyl-3-trityl glycerol (21c): The 3-trityl group was introduced according to the general procedure. TLC: R_{f}=0.11 (eluent E). ¹H NMR (CDCl₃), δ0.87 (t, J=7.2, 3H); 1.27 (br, 22H); 1.55 (m, 2H); 2.41 (br, 1H); 3.14 (m, 2H); 3.34-3.53 (m, 4H). 3.94 (m, 1H); 7.27 (m, 9H); 7.44 (m, 6H). MALDI-MS calcd for C₃₆H₅₁O₃⁺ [M+H]⁺ 531.39. Found 531.38.

1-Oleyl-3-trityl glycerol (21d): The 3-trityl group was introduced according to the general procedure. TLC: R_{f}=0.11 (eluent E). ¹H NMR (CDCl₃), δ0.88 (t, J=7.2, 3H); 1.28 (br, 22H); 1.56 (m, 2H); 2.0 (m, 4H); 2.42 (br, 1H); 3.20 (m, 2H); 3.36-3.54 (m, 4H); 3.96 (m, 1H); 5.35 (m, 2H); 7.25 (m, 9H); 7.45 (m, 6H). MALDI-MS calcd for C₄₀H₅₇O₃⁺ [M+H]⁺ 585.43. Found 585.44.

1-Stearyl-2-cholesterylcarbonoyl-3-trityl glycerol (22a): To a solution of 21a (2.4 g, 4 mmol) and DMAP (0.6 g) in dry ethanol-free chloroform (10 mL), was added dropwise the solution of cholesteryl chloroformate (2.2 g, 4.8 mmol) in chloroform (5 mL) at r.t. The reaction mixture was stirred at r.t. overnight. Then a mixture solvent of CHCl₃/MeOH/H₂O (40 mL/20 mL/30 mL) was added to the reaction mixture. The organic layer was dried over sodium sulfate, filtered, and evaporated. The crude product was purified by HPFC (0-10% ethyl acetate in hexane). TLC: R_{f}=0.43 (eluent E). ¹H NMR (CDCl₃), δ0.69 (s, 3H); 0.85-1.65 (m, 68H); 1.79-2.02 (m, 5H); 2.42 (m, 2H); 3.25 (m, 2H); 3.38 (m, 2H); 3.60 (m, 2H); 4.48 (m, 1H); 5.04 (m, 1H); 5.39 (d, J=4.4, 1H); 7.27 (m, 9H); 7.43 (m, 6H). MALDI-MS calcd for C₆₈H₁₀₃O₅⁺ [M+H]⁺ 999.78. Found 999.75.

1-Palmityl-2-cholesterylcarbonoyl-3-trityl glycerol (22b): This compound was synthesized according to the same procedure of 22a. TLC: R_{f}=0.43 (eluent E). ¹H NMR (CDCl₃), δ0.69 (s, 3H); 0.85-1.65 (m, 64H); 1.79-2.02 (m, 5H); 2.42 (m, 2H); 3.24 (m, 2H); 3.37 (m, 2H); 3.61 (m, 2H); 4.48 (m, 1H); 5.04 (m, 1H); 5.39 (d, J=4.4, 1H); 7.27 (m, 9H); 7.43 (m, 6H). MALDI-MS calcd for C₆₆H₉₉O₅⁺ [M+H]⁺ 971.75. Found 971.78.

1-Myristyl-2-cholesterylcarbonoyl-3-trityl glycerol (22c): This compound was synthesized according to the same procedure of 22a. TLC: R_{f}=0.43 (eluent E). ¹H NMR (CDCl₃), δ0.69 (s, 3H); 0.85-1.65 (m, 60H); 1.79-2.02 (m, 5H); 2.42 (m, 2H); 3.23 (m, 2H); 3.37 (m, 2H); 3.61 (m, 2H); 4.48 (m, 1H); 5.04 (m, 1H); 5.39 (d, J=4.4, 1H); 7.27 (m, 9H); 7.43 (m, 6H). MALDI-MS calcd for C₆₄H₉₅O₅⁺ [M+H]⁺ 943.72. Found 943.71.

1-Oleyl-2-cholesterylcarbonoyl-3-trityl glycerol (22d): This compound was synthesized according to the same procedure of 22a. TLC: R_{f}=0.43 (eluent E). ¹H NMR (CDCl₃), δ0.69 (s, 3H); 0.85-1.65 (m, 60H); 1.79-2.02 (m, 9H); 2.40 (m, 2H); 3.23 (m, 2H); 3.37 (m, 2H); 3.61 (m, 2H); 4.48 (m, 1H); 5.04 (m, 1H); 5.33-5.39 (m, 3H); 7.27 (m, 9H); 7.44. (m, 6H). MALDI-MS calcd for C₆₈H₁₀₁O₅⁺ [M+H]⁺ 997.77. Found 997.74.

1-Stearyl-2-cholesterylcarbonoyl glycerol (23a): This compound was synthesized according to the general procedure of removal of trityl group. TLC: R_{f}=0.63 (eluent D).

1-Palmityl-2-cholesterylcarbonoyl glycerol (23b): This compound was synthesized according to the general procedure of removal of trityl group. TLC: R_{f}=0.63 (eluent D).

1-Myristyl-2-cholesterylcarbonoyl glycerol (23c): This compound was synthesized according to the general procedure of removal of trityl group. TLC: R_{f}=0.63 (eluent D).

1-Oleyl-2-cholesterylcarbonoyl glycerol (23d): This compound was synthesized according to the general procedure of removal of trityl group. TLC: R_{f}=0.63 (eluent D).

1-Stearyl-2-cholesterylcarbonoyl-rac-glycero-3-phosphate (24a): This compound was synthesized according to the general procedure of phosphorylation. TLC: R_{f}=0.57 (eluent C).

1-Palmityl-2-cholesterylcarbonoyl-rac-glycero-3-phosphate (24b): This compound was synthesized according to the general procedure of phosphorylation. TLC: R_{f}=0.57 (eluent C).

1-Myristyl-2-cholestetylcarbonoyl-rac-glycero-3-phosphate (24c): This compound was synthesized according to the general procedure of phosphorylation. TLC: R_{f}=0.57 (eluent C).

1-Oleyl-2-cholesterylcarbonoyl-rac-glycero-3-phosphate (24d): This compound was synthesized according to the general procedure of phosphorylation. TLC: R_{f}=0.57 (eluent C).

1-Stearyl-2-cholesterylcarbonoyl-rac-glycero-3-phosphocholine (SML4a, SₑCh_{c}PC): This compound was synthesized according to the general procedure of phosphocholine synthesis. TLC: R_{f}=0.53 (eluent A). ¹H NMR (CDCl₃), δ0.68 (s, 3H); 0.85-1.65 (m, 66H); 1.84-2.05 (m, 5H); 2.36 (m, 2H); 3.39 (s, 9H); 3.44 (m, 2H); 3.61 (m, 2H); 3.83 (m, 2H); 4.01 (m, 2H); 4.35 (m, 2H); 4.44 (m, 1H); 4.98 (m, 1H); 5.39 (d, J=4.4, 1H). MALDI-MS calcd for C₅₄H₁₀₁NO₈P⁺ [M+H]⁺ 922.73. Found 922.73.

1-Palmityl-2-cholesterylcarbonoyl-rac-glycero-3-phosphocholine (SML4b, PₑCh_{c}PC): This compound was synthesized according to the general procedure of phosphocholine synthesis. TLC: R_{f}=0.53 (eluent A). ¹H NMR (CDCl₃), δ0.68 (s, 3H); 0.85-1.65 (m, 62H); 1.84-2.05 (m, 5H); 2.36 (m, 2H); 3.38 (s, 9H); 3.45 (m, 2H); 3.61 (m, 2H); 3.82 (m, 2H); 4.02 (m, 2H); 4.35 (m, 2H); 4.44 (m, 1H); 4.98 (m, 1H); 5.39 (d, J=4.4, 1H). MALDI-MS calcd for C₅₂H₉₇NO₈O⁺ [M+H]⁺ 894.69. Found 894.68.

1-Myristyl-2-cholesterylcarbonoyl-rac-glycero-3-phosphocholine (SML4c, MₑCh_{c}PC): This compound was synthesized according to the general procedure of phosphocholine synthesis. TLC: R_{f}=0.53 (eluent A). ¹H NMR (CDCl₃), δ0.68 (s, 3H); 0.85-1.65 (m, 58H); 1.84-2.05 (m, 5H); 2.37 (m, 2H); 3.39 (s, 9H); 3.44 (m, 2H); 3.61 (m, 2H); 3.83 (m, 2H); 4.02 (m, 2H); 4.35 (m, 2H); 4.44 (m, 1H); 4.98 (m, 1H); 5.39 (d, J=4.4, 1H). MALDI-MS calcd for C₅₀H₉₃NO₈P⁺ [M+H]⁺ 866.66. Found 866.64.

1-Oleyl-2-cholesterylcarbonyl-rac-glycero-3-phosphocholine (SML4d, OₑCh_{c}PC): This compound was synthesized according to the general procedure of phosphocholine synthesis. TLC: R_{f}=0.53 (eluent A). ¹H NMR (CDCl₃), δ0.69 (s, 3H); 0.85-1.65 (m, 58H); 1.84-2.05 (m, 9H); 2.37 (m, 2H); 3.38 (s, 9H); 3.45 (m, 2H); 3.62 (m, 2H); 3.84 (m, 2H); 4.02 (m, 2H); 4.35 (m, 2H); 4.45 (m, 1H); 4.98 (m, 1H); 5.35-5.39 (m, 3H). MALDI-MS calcd for C₅₄H₉₉NO₈P⁺ [M+H]⁺ 920.71. Found 920.71.

### Example 5

### Preparation of Lipids SML5a-5d

A synthetic scheme for the synthesis of lipids SML5a, SML5b, SML5c, and SML5d (referred to collectively as SML5a-d) is outlined in Scheme 5. This scheme is exemplified below by the detailed description of the synthesis of lipids SML5a-d.

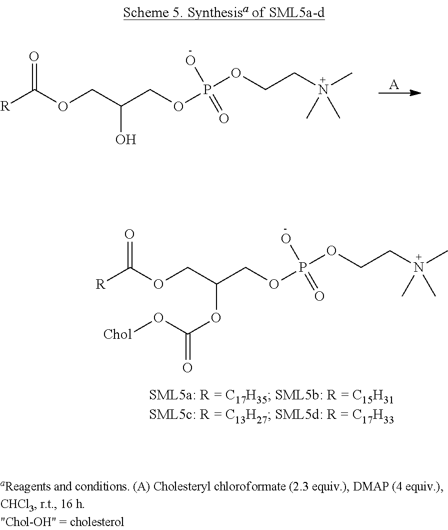

1-Stearoyl-2-cholesterylcarbonoyl-sn-glycero-3-phosphocholine (SML5a, SCh_{c}PC): To a solution of 1-stearoyl-2-hydroxy-sn-glycero-phosphocholine (1 g, 1.91 mmol) and DMAP (1 g) in ethanol-free dry chloroform (50 mL), was added dropwise the chloroform solution (10 mL) of cholesteryl chloroformate (2 g, 4.45 mmol) at r.t. After 16 h reaction at r.t., solvent was evaporated and the residue was purified by HPFC(CHCl₃ to CHCl₃-MeOH—H₂O 65/25/4). TLC: R_{f}=0.54 (eluent C). ¹H NMR (CDCl₃/MeOH-d₄/pyridine-d₅, 10:2:1), δ0.68 (s, 3H); 0.85-1.65 (m, 66H); 1.84-2.05 (m, 5H); 2.33 (t, J=7.6 Hz, 2H); 2.39 (m, 2H); 3.28 (s, 9H); 3.67 (m, 2H); 4.10 (m, 2H); 4.24 (m, 1H); 4.32 (m, 2H); 4.47 (m, 2H); 5.11 (m, 1H); 5.41 (d, J=4.4, 1H). MALDI-MS calcd for C₅₄H₉₉NO₉P⁺ [M+H]⁺ 937.61. Found 937.67.

1-Palmitoyl-2-cholesterylcarbonoyl-sn-glycero-3-phosphocholine (SML5b, PCh_{c}PC): This compound was synthesized according to the same procedure of 5a. TLC: R_{f}=0.54 (eluent C). ¹H NMR (CDCl₃/MeOH-d₄/pyridine-d₅, 10:2:1), δ0.68 (s, 3H); 0.85-1.65 (m, 62H); 1.84-2.05 (m, 5H); 2.31 (t, J=7.6, 2H); 2.36 (m, 2H); 3.27 (s, 9H); 3.66 (m, 2H); 4.08 (m, 2H); 4.23 (m, 1H); 4.30 (m, 2H); 4.44 (m, 2H); 5.08 (m, 1H); 5.40 (d, J=4.4, 1H). MALDI-MS calcd for C₅₂H₉₅NO₉P⁺ [M+H]⁺ 908.67. Found 908.67.

1-Myristoyl-2-cholesterylcarbonoyl-sn-glycero-3-phosphocholine (SML5c, MCh_{c}PC): This compound was synthesized according to the same procedure of 5a. TLC: R_{f}=0.54 (eluent C). ¹H NMR (CDCl₃/MeOH-d₄/pyridine-d₅, 10:2:1), δ0.69 (s, 3H); 0.85-1.65 (m, 58H); 1.84-2.05 (m, 5H); 2.33 (t, J=7.6, 2H); 2.39 (m, 2H); 3.27 (s, 9H); 3.67 (m, 2H); 4.09 (m, 2H); 4.24 (m, 1H); 4.31 (m, 2H); 4.45 (m, 2H); 5.09 (m, 1H); 5.40 (d, J=4.4, 1H). MALDI-MS calcd for C₅₂H₉₅NO₉P⁺ [M+H]⁺ 908.67. Found 908.67.

1-Oleoyl-2-cholesterylcarbonoyl-sn-glycero-3-phosphocholine (SML5d, OCh_{c}PC): This compound was synthesized according to the same procedure of 5a. TLC: R_{f}=0.54 (eluent C). ¹H NMR (CDCl₃/MeOH-d₄/pyridine-d₅, 10:2:1), δ0.69 (s, 3H); 0.85-1.65 (m, 58H); 1.84-2.06 (m, 9H); 2.33 (t, J=7.6, 2H); 2.39 (m, 2H); 3.28 (s, 9H); 3.68 (m, 2H); 4.10 (m, 2H); 4.24 (m, 1H); 4.32 (m, 2H); 4.47 (m, 2H); 5.10 (m, 1H); 5.36 (m, 2H); 5.40 (d, J=4.4, 1H). MALDI-MS calcd for C₅₄H₉₇NO₉P⁺ [M+H]⁺ 934.69. Found 934.68.

### Example 6

### Preparation of Lipids SML6a-d

A particular synthetic scheme for the synthesis of lipids SML6a, SML6b, SML6c, and SML6d (referred to collectively as SML6a-d) is outlined in Scheme 6. This scheme is exemplified below by the detailed description of the synthesis of lipids SML6a-d.

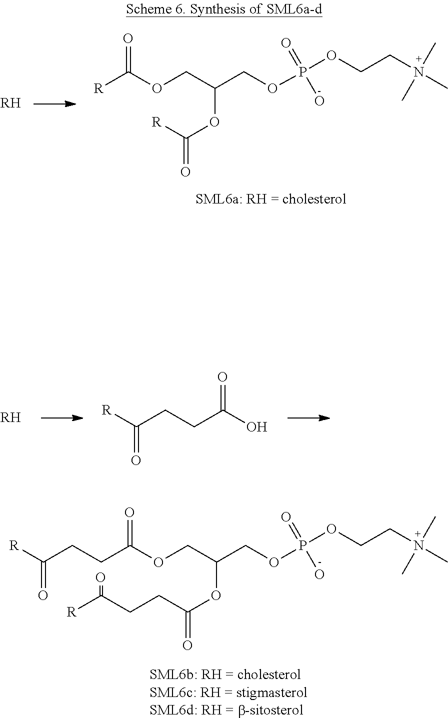

1,2-Dicholesterylcarbonoyl-sn-glycero-3-phosphocholine (SML6a, DCh_{c}PC): Glycerophosphocholine (0.514 g, 2 mmol) and sodium tetraphenylborate (0.719 g, 1.05 equiv.) were dissolved in 15 mL methanol. Solvent was evaporated, and the residue was azeotropically dried with toluene. The dried solid was then dissolved in anhydrous pyridine (60 mL), followed by the addition of 4,4-dimethylaminopyridine (0.732 g, 6 mmol). Cholesteryl chloroformate (2.70, 6 mmol) was added into the reaction mixture in portion at r.t. with vigorous stirring. The reaction flask was then purged with nitrogen and kept under dark for 3 days. Volatiles were rotary evaporated. The crude product was dissolved in chloroform-methanol (2:1, 150 mL), and washed with 50 mL distilled water. The organic layer was dried by anhydrous sodium sulfate, filtered and evaporated. The residue was applied to the HPFC for further purification (CHCl₃ to CHCl₃/MeOH/H₂O (65/25/4)). Yield, 0.38 g (17.7%). TLC: R_{f}=0.4 (eluent A). ¹H NMR (CDCl₃), δ0.68 (s, 6H); 0.85-1.65 (m, 66H); 1.81-2.06 (m, 10H); 2.37 (m, 4H); 3.42 (s, 9H); 3.89 (m, 2H); 4.06 (m, 2H), 4.26 (m, 2H); 4.36 (m, 2H); 4.44 (m, 2H); 5.04 (m, 1H); 5.38 (d, J=4.4, 2H). MALDI-MS calcd for C₆₄H₁₀₉NO₁₀P⁺ [M+H]⁺ 1082.79. Found 1082.73.

1,2-Dicholesterylhemisuccinoyl-sn-glycero-3-phosphocholine (SML6b, DCHEMSPC): Glycerophosphocholine (1.03 g, 4 mmol) and sodium tetraphenylborate (1.33 g, 1 equiv.) were dissolved in 30 mL methanol. Solvent was evaporated, and the residue was azeotropically dried with toluene. The dried solid was then dissolved in anhydrous pyridine (120 mL), followed by the addition of 4,4-dimethylaminopyridine (0.9 g) and cholesteryl hemisuccinate (4.86 g, 10 mmol). The mixture was gently warmed up to dissolve the solid completely. Dicyclohexylcarbodiimide (2.32 g, 11 mmol) was added to the reaction mixture after it was cooled to the room temperature. The mixture was stirred under nitrogen at room temperature for 3 days. Volatiles were rotary evaporated, and the residue was dissolved in chloroform-methanol (2:1, 300 mL), washed with 80 mL distilled water. The organic layer was dried over anhydrous sodium sulfate, filtered, evaporated, and applied to the HPFC for further purification. (CHCl₃ to CHCl₃/MeOH/H₂O (65/25/4)). Yield, 2.3 g (48%). TLC: R_{f}=0.42 (eluent A). ¹H NMR (CDCl₃), δ0.68 (s, 6H); 0.85-1.65 (m, 66H); 1.81-2.06 (m, 10H); 2.30 (m, 4H); 2.60 (m, 8H); 3.39 (s, 9H); 3.85 (m, 2H); 3.99 (m, 2H), 4.23 (m, 2H); 4.35 (m, 2H); 4.58 (m, 2H); 5.22 (m, 1H); 5.37 (d, J=4.4, 2H). MALDI-MS calcd for C₇₀H₁₁₆NO₁₂P⁺ [M+H]⁺ 1194.84. Found 1194.79.

1,2-Distigmasterylhemisuccinoyl-sn-glycero-3-phosphocholine (SML6c, DStigHSPC): This compound was synthesized according to the same procedure of SML6b. TLC: R_{f}=0.36 (eluent A). Structure was confirmed by NMR and MALDI-MS.

1,2-Disitosterylhemisuccinoyl-sn-glycero-3-phosphocholine (SML6d, DSitoHSPC): This compound was synthesized according to the same procedure of SML6b. TLC: R_{f}=0.40 (eluent A). Structure was confirmed by NMR and MALDI-MS.

### Example 7

### Preparation of Prodrug Lipids SML7a-b

The retinoic acid prodrugs SML7a and SML7b (referred to collectively as SML7a-b) are synthesized according to the similar synthetic route shown in example 3. A brief synthetic route for the synthesis of SML7a-b was outlined in Scheme 7. This scheme is exemplified below by the description of the synthesis of lipid SML7a-b.

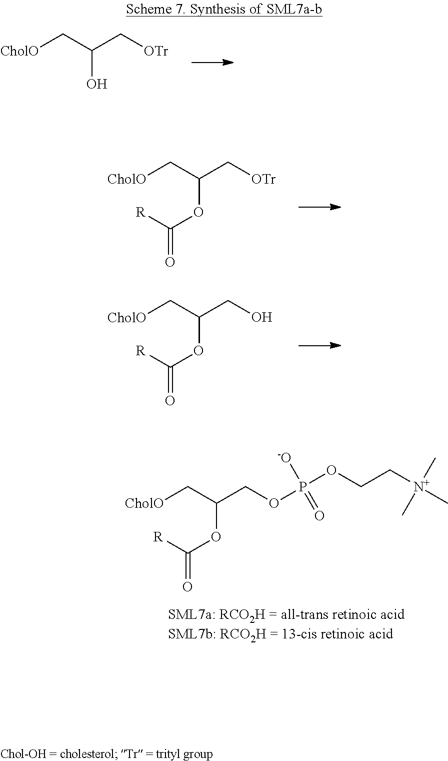

The selectively protected cholesteryl glycerol intermediate was synthesized according to the same procedure described in the above example 3. Retinoic acid was then coupled to the sn-2 hydroxyl group through an ester bond followed by the removal of the trityl group (Tr) from 3-hydroxyl group. Then the intermediate was immediately converted to the corresponding phosphocholine according to the standard procedure described in the general protocol of the examples. The final products were purified by HPFC and the structures were confirmed by TLC, NMR, and MALDI-MS.

### Example 8

### Preparation of Lipid SML8a-f

A synthetic scheme for the synthesis of lipids SML8a, SML8b, SML8c, SML8d, SML8e, and SML8f (referred to collectively as SML8a-f) is outlined in Scheme 8. This scheme is exemplified below by the detailed description of the synthesis of lipid SML8a-f.

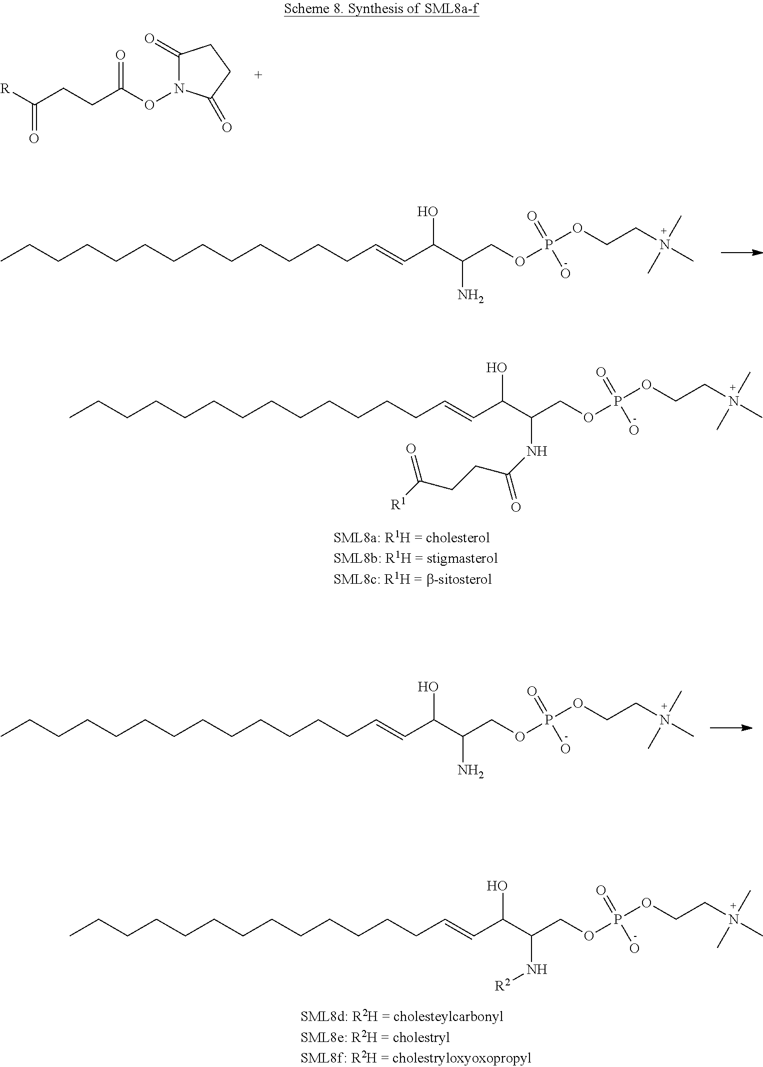

The sterol-lysosphingomyelin conjugates (SML8a-c) were synthesized according to the synthetic route shown in scheme 8. First the hemisuccinates of sterol was activated with N-hydroxysuccinimide by DCC. Then the activated ester of sterol was coupled to the amino group of lyso-sphingomyelin. The final products were further purified by HPFC.

SML8d-f were synthesized by the reaction of lyso-sphingomyelin and cholesteryl chloroformate, cholesteryl tosylate, and cholesteryl acrylate, respectively.

Cholesteryloxy-4-oxobutanamido-3-hydroxyoctadec-4-enyl 2-(trimethylammonio)ethyl phosphate (SML8a): To a solution of lyso-sphingomyelin (25 mg, 53 μmol) in ethanol-free dry chloroform (6 mL) at room temperature, were added succinimidyl cholesteryl succinate (62 mg, 106 μmol) and DMAP (10 mg). The reaction mixture was stirred at r.t. for 24 h. Solvent was evaporated. The residue was applied to HPFC for purification (CHCl₃ to CHCl₃-MeOH—H₂O 65/25/4). TLC: R_{f}=0.33 (eluent C). ¹H NMR (CDCl₃), δ0.68 (s, 3H); 0.85-0.92 (m, 14H); 1.00-1.15 (m, 10H); 1.25 (br, 28H); 1.42-1.62 (m, 6H); 1.84-2.05 (m, 7H); 2.29 (m, 2H); 2.51 (m, 4H); 2.68 (br, 1H); 3.28 (s, 9H); 3.72 (m, 1H); 4.01 (m, 4H); 4.28 (m, 2H); 4.58 (m, 2H); 5.34 (m, 1H); 5.44 (d, J=4.2, 1H); 5.68 (m, 1H); 7.51 (br, 1H). MALDI-MS calcd for C₅₄H₉₈N₂O₈P⁺ [M+H]⁺ 933.72. Found 933.68.

2-cholesteryloxycarbonylamino-3-hydroxyoctadec-4-enyl 2-(trimethylammonio)ethyl phosphate (SML8d): To a solution of lyso-sphingomyelin (25 mg, 53 μmol) and DMAP (20 mg) in ethanol-free dry chloroform (4 mL) at room temperature, was added cholesteryl chloroformate (48 mg, 106 μmol) in 2 mL dry ethanol free chloroform. After 24 h stirring at r.t., solvent was evaporated. The residue was applied to HPFC for purification (CHCl₃ to CHCl₃-MeOH—H₂O 65/25/4). TLC: R_{f}=0.41 (eluent C). ¹H NMR (CDCl₃), δ0.68 (s, 3H); 0.85-0.92 (m, 14H); 1.00-1.15 (m, 10H); 1.25 (br, 28H); 1.47-1.528 (m, 6H); 1.84-2.05 (m, 7H); 2.29 (m, 2H); 2.65 (br, 1H); 3.3 (s, 9H); 3.61 (m, 1H); 3.89 (m, 4H); 4.28 (m, 4H); 5.34 (m, 1H); 5.48 (d, J=4.4, 1H); 5.73 (m, 1H); 6.16 (br, 1H). MALDI-MS calcd for C₅₁H₉₄N₂O₇P⁺ [M+H]⁺ 877.69. Found 877.76.

### Example 9

### Preparation of Lipid SML9a-c

A particular synthetic scheme for the synthesis of lipid SML9a-c is outlined in Scheme 9. This scheme is exemplified below by the detailed description of the synthesis of lipid SML9a-c.

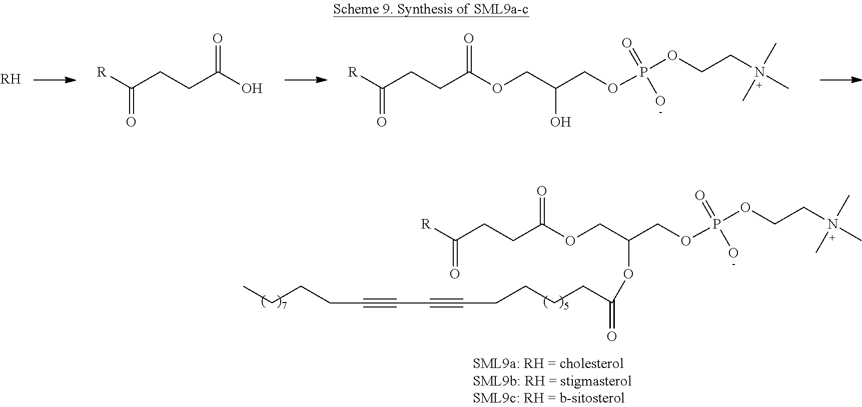

The polymerizable SML lipids (SML9a-c) may be synthesized according to the synthetic route shown in scheme 9. First hemisuccinate of sterol can be selectively coupled to the sn-1 position of glycerophosphocholine. Then 10,12-tricosodiynoic acid can be conjugated to the sn-2 position affording the final product. Crude products can be purified by HPFC.

### Example 10

### Preparation of Branched Lipid SML10a-f

A particular synthetic scheme for the synthesis of lipid SML10a-f is outlined in Scheme 10. This scheme is exemplified below by the detailed description of the synthesis of lipid SML10a-f.

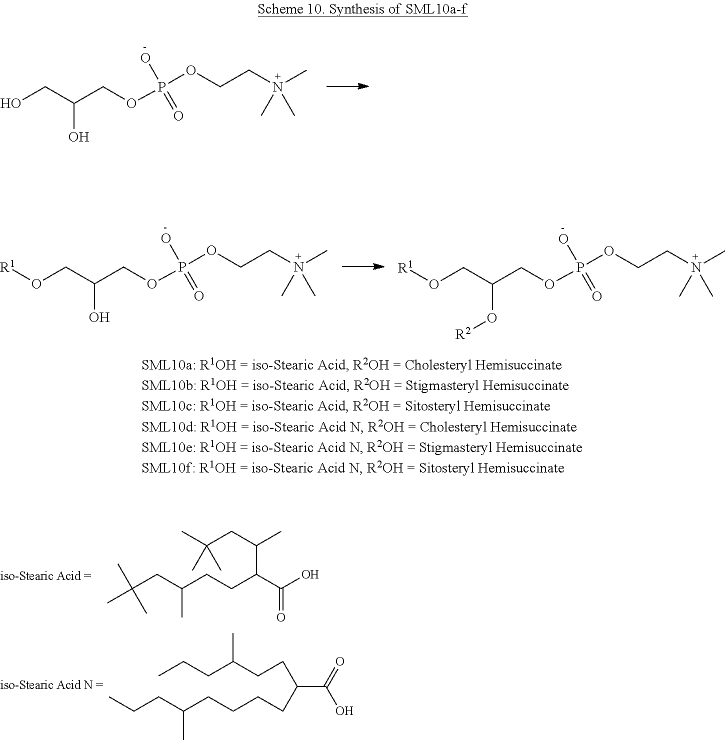

The branched iso-stearic acid was conjugated to the sn-1 position of glycerophocholine with a similar synthetic method of SML6b described in the example 6 but with a different molar ratio of starting materials. The mono-iso-stearoyl phosphocholine was then separated by HPFC, and conjugated to hemisuccinate of sterol by dicyclohexylcarbodiimide. The final product was purified by HPFC.

### Example 11

### Preparation of Branched Lipid SML11a-f

A particular synthetic scheme for the synthesis of lipid SML11a-f is outlined in Scheme 11. This scheme is exemplified below by the detailed description of the synthesis of lipid SML11a-f.

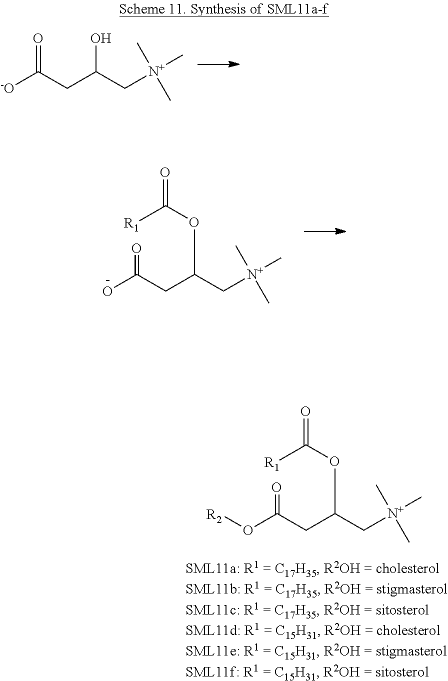

The target SML lipids SML11a-f can be synthesized in two steps using carnitine as the starting material. First, the activated aliphatic acid is conjugated to the hydroxyl group by ester bond. Then sterol is connected to the carboxyl group also through the ester linkage. The final product may be purified by HPFC.

### Example 12

### Preparation of SML Having Tyrosine SML12a-f

A particular synthetic scheme for the synthesis of lipid SML12a-f is outlined in Scheme 12. This scheme is exemplified below by the detailed description of the synthesis of lipid SML12a-f.

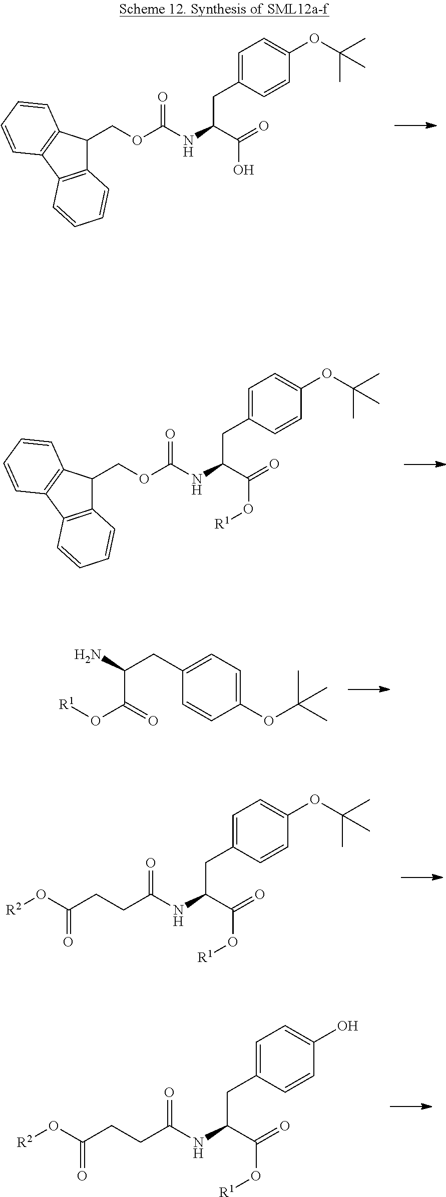

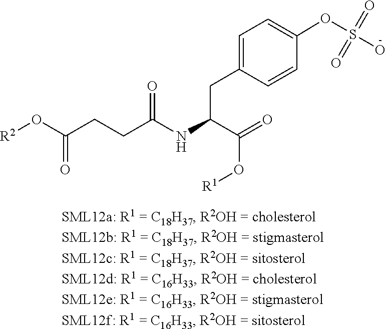

The sterol-modified amphiphilic lipid SML12a-f may be synthesized according to the route shown in scheme 12. First, aliphatic alcohol is conjugated to the carboxyl group of selectively protected tyrosine followed by the removal of Fmoc group with piperidine. Sterol hemisuccinate is then coupled with the amino group. After the removal of the tert-butyl group, the phenol group is sulphated. The phenol group may also be converted to phosphate, phosphonate, sulphonate, borate, or hydrazone. This route provides a synthetic pathway to other amino acid based sterol-modified lipids where the appropriately protected amino acid is used as the branching core.

### Example 13

### Preparation of Reduction-Sensitive SML13a-h

The reduction-sensitive SML lipids are designed to have a reducible bond in the molecule that can be cleaved selectively under the reducing biological environment such as found in the cell cytosol. This will destabilize the lipid particle and facilitate the release of the drug selectively. The disulfide bond can be placed at different parts of the molecules such as the side chain (SML13a-h), or the head group (SML13i-k). The synthesis of lipid SML13a-k is outlined in Scheme 13.1, Scheme 13.2, and Scheme 13.3. The schemes are exemplified below by the detailed description of the synthesis of lipid SML13a-k.

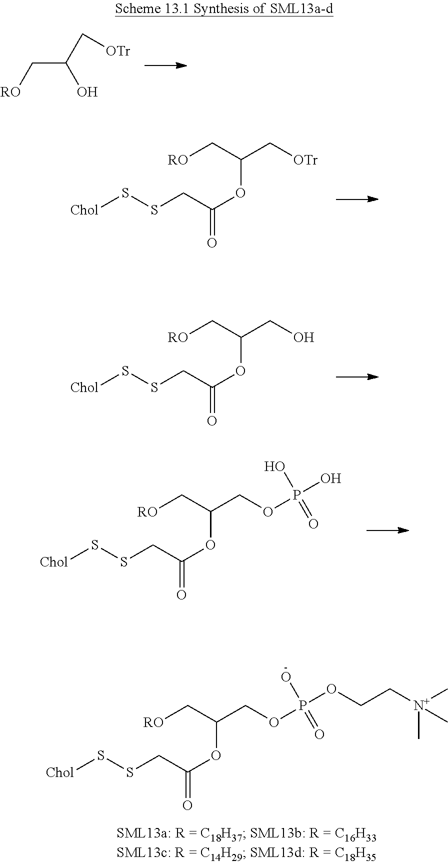

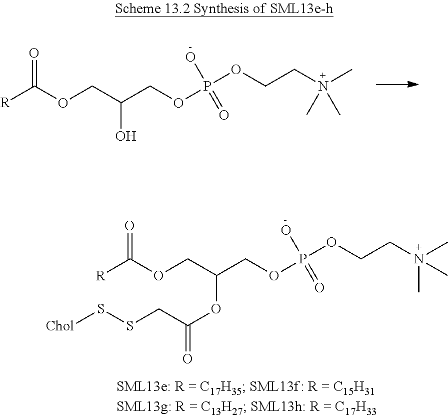

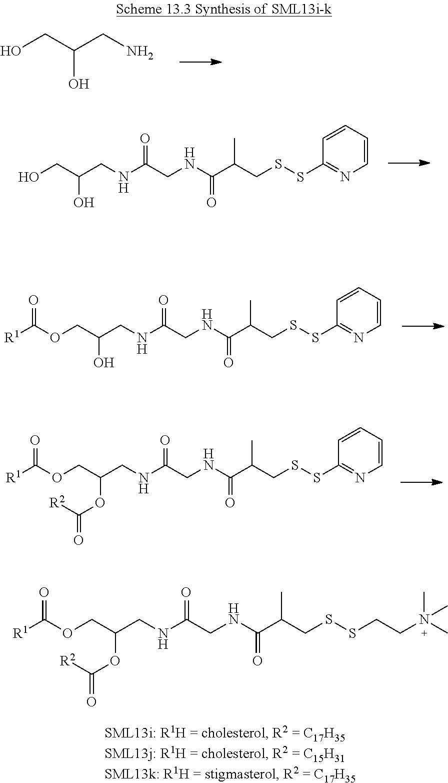

SML13a-d can be synthesized by the similar method of SML4a-d described in the above Example 4. Instead of cholesteryl chloroformate, 2-cholesteryldisulfanyl acetic acid is conjugated to the sn-2 hydroxyl group. SML13e-h can be synthesized according to the same method of SML5a-d by replacing cholesteryl chloroformate with 2-cholesteryldisulfanyl acetic acid. In the synthesis of SML13i-k, a 2-thiopyridine activated disulfide moiety is connected to the amino group first. Then the sterol is conjugated to the primary alcohol followed by the attachment of fatty acid to the secondary alcohol. Finally, a cationic head group is introduced by the disulfide exchange reaction. SML13i-k are reduction-sensitive cationic SML compounds. They may be very useful for gene and siRNA delivery.

### Example 14

### Preparation of SML Bearing Azide at the End of Head Group SML14a-f

The design of targeted liposomes depends in large part on the development of well-controlled bioconjugation reaction which in most cases involve the coupling of ligands to the surface of pre-formed vesicles that carry functionalized lipid anchors. Among the many conjugation methods available, the most popular ones involve the reaction of thiol-containing ligands with anchors carrying thiol-reactive functions such as maleimide, bromoacetyl, or 2-pyridyldithio linkages, generating thioether or disulfide bonds, or azide, cyclizing with alkynes by “click-chemistry”. Use of end-functionalized SMLs can be advantageous as an anchorage for the targeting ligands or surface labels since they are less likely to phase separate or partition out of the bilayer than conventional lipids. SML compounds SML14a-f are some model compounds having an azide group at the end of head group that can be used to attach ligands or biomarkers through “click-chemistry”. Other functional groups, such as the propargyl group may be introduced in a similar way. A particular synthetic scheme for the synthesis of lipid SML14a-f is outlined in Scheme 14. This scheme is exemplified below by the detailed description of the synthesis of lipid SML14a-f.

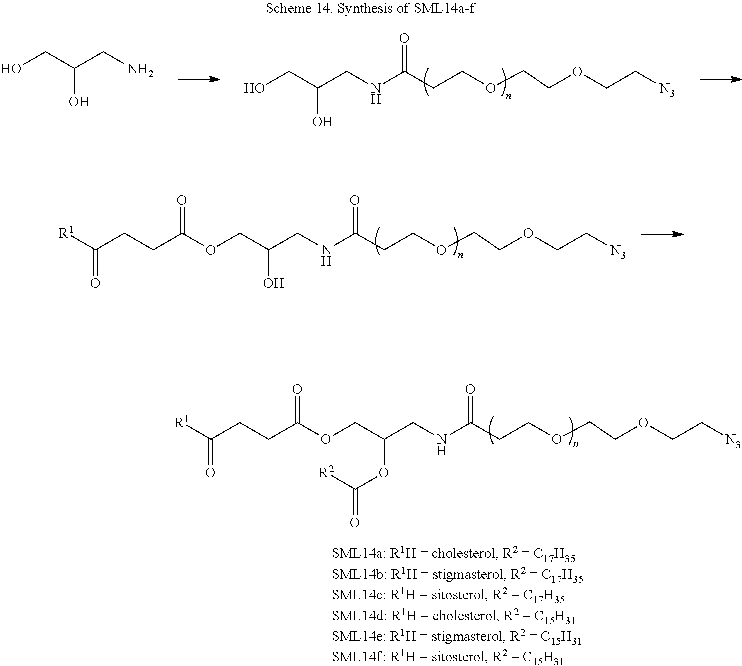

The synthesis of SML14a-f is straightforward. First, the azide-functionalized polyethylene oxide is attached to the more reactive amine. Then sterol hemisuccinate is conjugated to the primary alcohol. In the final step, the fatty acid is connected to the least reactive secondary alcohol. Due to the difference of reactivity of the functional groups, protective groups are not necessary.

### Example 15

### Differential Scanning Calorimetery of Selected SMLs

Specific SMLs were used as the model compounds to study the properties of the SML compounds. Phase behavior is one of the most important properties of lipid bilayers and is associated with various biological functions of cell membranes. Addition of free cholesterol to the phospholipid bilayer will change the phase behavior of the bilayer due to the mixing of free cholesterol with the glycerolipids and sphingolipids as well as the effect the isoprenol tail of free cholesterol exerts on the acyl chain packing in the bilayer. Using differential scanning calorimetry it was shown that the SMLs exert a similar effect as free cholesterol on acyl chain packing in bilayers composed of synthetic lipids. Accordingly, SMLs containing C-16 chain with various linkages (SML1b-5b) and SMLs of one group with different chains (SML5a-5d) were chosen for the DSC study.

Differential scanning calorimetry (DSC) studies were carried out on an upgraded high-temperature MC-DSC 4100 calorimeter (calorimetry Sciences Corp., Lindon, Utah) with three reusable Hastelloy sample ampoules and a reference ampoule. Data was collected typically over a range of 5-85° C. at 0.5° C./min with Milli-Q® water as the reference. The CpCalc 2.1 software package from CSC was used to convert the raw data into molar heat capacity (MHC). Data were then exported to Origin 6.0 (Microcal, Northampton, Mass.) for further process and calculation. Liposomes used for DSC measurement were prepared by hydrating the lipid film (10 μmol) in Milli-Q® water (200 μL) at 65° C. under argon for 15 min with intermittent vortex. Samples were then cooled to room temperature, degassed, and loaded into the sample ampoule using gas-tight Hamilton® syringe (100 μL per sample). Samples were scanned through a heating-cooling-heating cycle and the second heating scan data was used for analysis.

In a conventional lipid mixture of free cholesterol and diacyl phospholipid, the molar percentage of cholesterol (Chol) in the total lipids is calculated by the following formula:

Chol %=n_{chol}/(n_{chol}+n_{diacyl})×00, wherein:

n_{chol} refers to the moles of cholesterol.

n_{diacyl} refers to the moles of diacyl lipids.

When an m-SML (monosterol SML) is mixed with a diacyl phospholipid, there is an additional acyl chain in m-SML that is accounted for in the calculation of total moles of diacyl lipids. The equivalent sterol molar percentage in the mixture of m-SML and diacyl lipid is calculated according to the method of conventional lipid mixture with the following formula:

Sterol %=n_{m-SML}/(1.5×n_{m-SML}+N_{diacyl})×100, wherein:

n_{m-SML} refers to the moles of m-SML.

n_{diacyl} refers to the moles of diacyl lipids.

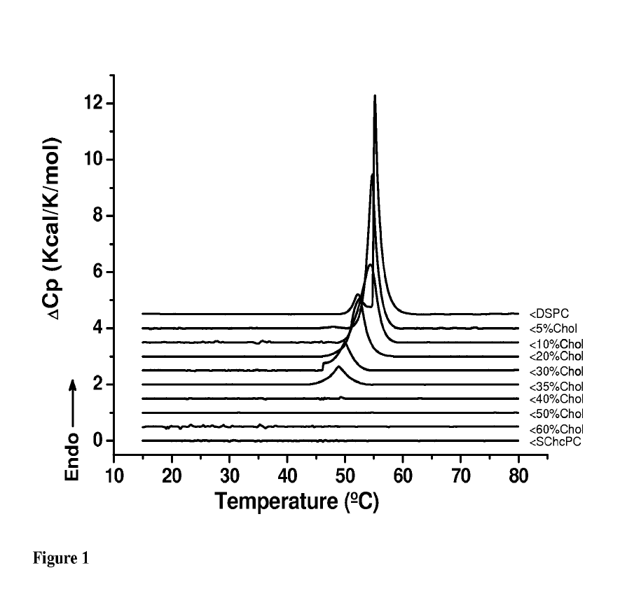

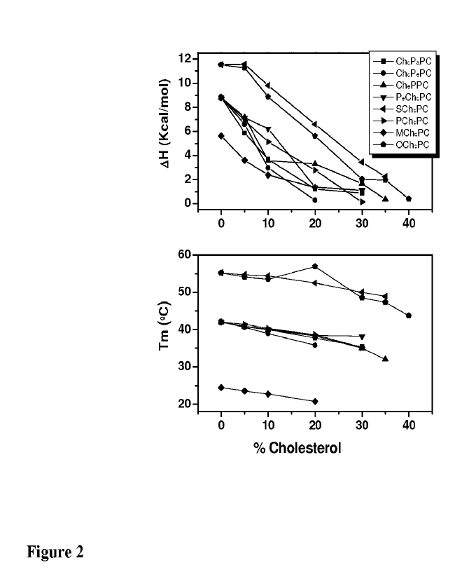

For example, a pure m-SML has one sterol and one acyl chain, thus the equivalent free sterol percentage is 1/1.5×100=67. In a 1:1 mole mixture of m-SML and diacyl lipid, the equivalent free sterol percentage is 1/(1.5+1)×100=40. The same calculation method was applied to all other m-SML formulations. DSC results are shown in FIG. 1 and FIG. 2.

In FIG. 1, percentage values of equivalent cholesterol (Chol) refer to mole percentages. DSPC refers to distearoylphosphatidylcholine; SChcPC refers to a SML having a single sterol, specifically 1-Stearoyl-2-cholesterylcarbonoyl-sn-glycero-3-phosphocholine (SML5a).

In FIG. 2, the transition temperature and enthalpy of SML/diacyl lipid mixtures with various mole percentages of free cholesterol. SMLs were mixed with diacyl lipids of the same chain length. The SMLs tested in FIG. 2 were as follows:
1-Cholesterycarbonoyl-2-palmitylcarbamoyl-sn-glycero-3-phosphocholine (SML1b, Ch_{c}PₑPC)
1-Cholesterylcarbonoyl-2-palmityl-rac-glycero-3-phosphocholine (SML2b, Ch_{c}PₑPC)
1-Cholesteryl-2-palmitoyl-rac-glycero-3-phosphocholine (SML3b, ChₑPPC)
1-Palmityl-2-cholesterylcarbonoyl-rac-glycero-3-phosphocholine (SML4b, PₑCh_{c}PC):
1-Stearoyl-2-cholesterylcarbonoyl-sn-glycero-3-phosphocholine (SML5a, SCh_{c}PC)
1-Palmitoyl-2-cholesterylcarbonoyl-sn-glycero-3-phosphocholine (SML5b, PCh_{c}PC
1-Myristoyl-2-cholesterylcarbonoyl-sn-glycero-3-phosphocholine (SML5c, MCh_{c}PC):
1-Oleoyl-2-cholesterylcarbonoyl-sn-glycero-3-phosphocholine (SML5d, OCh_{c}PC)

The DSC thermogram of SCh_{c}PC and its mixtures with DSPC (FIG. 1) was the typical result for most of the SMLs tested. The major transition temperature (Tm) and enthalpy (ΔH) were plotted against the percentage of the cholesterol in the lipid mixture (FIG. 2). There was no detectable phase transition in the temperature range of 10-80° C. for all the SMLs tested no matter the chemical structure of the SMLs. Thus, a homogeneous liquid ordered bilayer of high cholesterol concentration (about 67%) is achievable if cholesterol and aliphatic chain are held close to each other. In the living cells, similar domain could be formed on the plasma membrane if the membrane protein has strong affinity for both lipid chain and cholesterol.

As with addition of free cholesterol, the addition of SML to the corresponding diacyl lipids broadened the transition peak, and decreased both the transition temperature and enthalpy. This effect was concentration dependent and eventually led to the elimination of the phase transition when cholesterol reached certain concentration.

Although the condensing effects of SMLs of different linkages are similar, there are noticeable differences on Tm and ΔH especially between Ch_{c}PₑPC and PₑCh_{c}PC which are isomers. Ch_{c}PₑPC was able to rapidly lower the Tm and ΔH and eliminate the phase transition completely at 30% equivalent cholesterol while other SMLs with same chain length need at least 35% equivalent cholesterol. It seems that the 2-ether linkage of Ch_{c}PₑPC is responsible for the difference by comparing Ch_{c}PₑPC with Ch_{c}PₐPC and PₑCh_{c}PC. It is also noticed that the addition of OCh_{C}PC to DSPC resulted in very broad transition peaks when cholesterol was no more than 20%. This is significantly different from the effect of SCh_{C}PC on DSPC (FIG. 1), and may be the result of chain mismatch between the unsaturated oleoyl chain and the saturated stearoyl chain.

### Example 16

### Encapsulation of Calcein Payload in Liposome

The fluorescent dye calcein (2.49 g, 4 mmol) was dissolved in Tris-HCl buffer (10 mM, pH 7.5, 6 mL) after the addition of 50% sodium hydroxide (695 μL, 13.2 mmol). This stock solution was then loaded on a Sephadex LH-20 column (2.5 cm×40 cm) and eluted with Tris-HCl buffer (10 mM, pH 7.5). The concentration of pooled fraction of calcein was determined by measuring the absorbance (494 nm) of diluted sample at pH 9. The purified calcein (56 mM) was then encapsulated into the liposomes for the leakage assay. Generally, the dry lipid film (10 μmol) of given formulation was hydrated in 1 mL calcein containing buffer at 60° C. under argon for 15 min with intermittent vortex. Then the sample was extruded through a polycarbonate membrane 11 times at 60° C. followed by passing a Sephadex G-50 column with the corresponding isosmotic eluent. The pooled liposome fractions were then analyzed to determine the calcein concentration, and diluted to the linear fluorescence range for leakage studies discussed below.

### Example 17

### Osmotic Stress Induced Leakage

One advantageous property of the SML-containing liposomes is resistance of leakage of liposomal contents (e.g., drugs) from the liposome. The inclusion of SML in the lipid bilayer will generally make the liposome less leaky in vitro. Testing the leakage of liposomes under osmotic pressure has been proven to be an effective method to evaluate the elastic deformation and critical failure of lipid membranes. When liposomes are subjected to high osmotic pressure, the membrane will swell and burst at the critical point to rapidly release the contents. Vesicle will then reseal into a mechanic stable structure once sufficient contents have been expelled. Thus the osmotic leakage profile is based on a quick equilibrium under the artificial osmotic gradients.

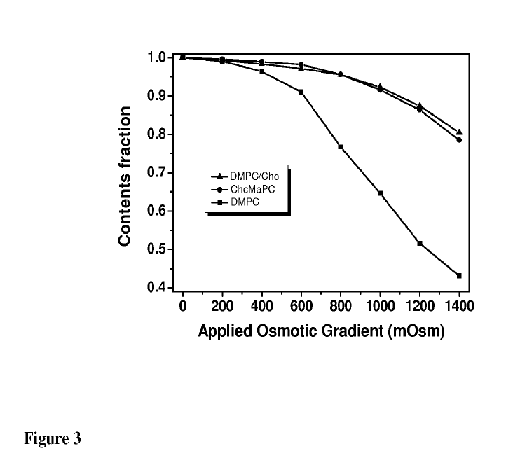

Liposomes used for this study were prepared according to the above mentioned method (Example 11) with high concentration content (56 mM calcein, 10 mM Tris, 711 mM NaCl), extruded through 100 nm membrane, and eluted with the isosmotic buffer (50 mM HEPES, 775 mM NaCl). Liposome of 1,2-dimyristoyl-sn-glycero-phosphocholine (DMPC) alone and DMPC and cholesterol (1:1 molar ratio) was used as the positive control in the leakage study of the SML compound Ch_{c}MₐPC (1-Cholesterylcarbonoyl-2-myristylcarbamoyl-sn-glycero-3-phosphocholine; SML1c) liposome. Solutions of various osmotic concentrations were prepared by mixing the calcein free isosmotic buffer (1600 mOsm) and a 50 mOsm dilution buffer (50 mM HEPES). Liposomes were then exposed to solutions of various osmotic concentrations by mixing 10 μL of liposome with 990 μL testing buffer at 37° C. Fluorescence signal at 517 nm (ext.: 494 nm) was read after 5 min equilibration by using a Quantech™ fluorometer (Barnstead/Thermolyne, Dubuque, Iowa). Liposomes were then lysed by adding 100 μL 10% Triton X-100 to release calcein completely. The fluorescence of the total calcein was measured and used as 100% signal (F_{100%}). The fraction of calcein remaining in the liposome before lysis was defined as 1−(F_{signal}−F_{blank})/(F_{100%}−F_{blank}), where F_{signal} is the fluorescence intensity of the sample and F_{blank} is the fluorescence intensity of liposome in the isosmotic buffer. Results are shown in FIG. 3.

The leakage of Ch_{c}MₐPC and DMPC/Cholesterol (1:1) under the gradient of osmotic pressure was monitored at 37° C. (FIG. 3). Both liposomes exhibited similar osmotic leakage profiles and good stability compared with the DMPC liposomes. Other SMLs have shown similar osmotic leakage profiles to that of Ch_{c}MₐPC. SML liposomes thus maintained their contents at least s as well as the corresponding cholesterol/diacyl lipid mixtures under the osmotic gradients tested.

### Example 18

### Assessment of SML-Containing Liposomes to Leakage in 30% Fetal Bovine Serum

The physiological environment presents another challenge for in vivo liposome drug delivery, namely the propensity of serum protein and biological membranes to extract free cholesterol from the liposome bilayer, resulting in leakage. This interaction can be greatly reduced by shielding the liposome surface with PEG (polyethylene glycol). The stability and resistance of SML liposomes was tested.

Calcein was encapsulated into the liposome by the method described above. Liposomes were extruded through 200 nm membrane and the free calcein was removed by passing the liposomes through the Sephadex G-50 column using HEPES buffer (10 mM HEPES, 140 mM NaCl, pH 7.4) as the isomotic eluent. Conventional liposome formulations containing 40% cholesterol (mole percent) were used as the control in the long term leakage assay. An aliquot of liposome sample (20-50 μL) was diluted by 30% fetal bovine serum to a total volume of 2 mL. Samples were then sealed in the glass tube and incubated at 37° C. Fluorescence intensities of samples were monitored at different time points and the fraction of calcein remaining in the liposome was determined by the similar method of osmotic stress induced leakage. The SMLs tested included:
1-Cholesterylcarbonoyl-2-myristylcarbamoyl-sn-glycero-3-phosphocholine (SML1c, Ch_{c}MₐPC)
1-Stearoyl-2-cholesterylcarbonoyl-sn-glycero-3-phosphocholine (SML5a, SCh_{c}PC
1-Cholesteryl-2-myristoyl-rac-glycero-3-phosphocholine (SML3c, ChₑMPC)
1-Myristoyl-2-cholesterylcarbonoyl-sn-glycero-3-phosphocholine (SML5c, MCh_{c}PC)

Liposomes of DSPC with free cholesterol (DSPC/Chol) and DMPC with free cholesterol (DMPC/Chol) served as controls.

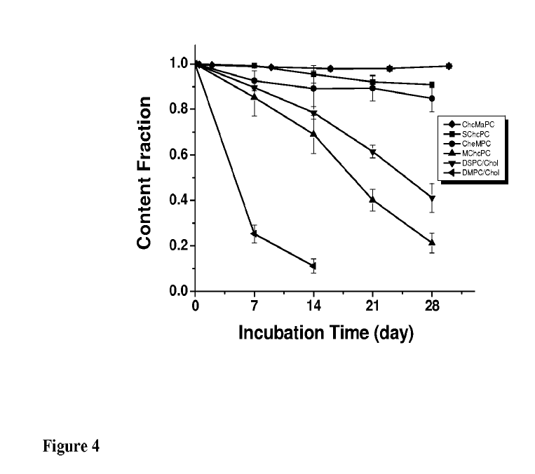

As shown in the results presented in FIG. 4, the Ch_{c}MₐPC liposomes remained intact while the contents of DSPC/cholesterol liposome were released gradually over the next 4 weeks. Liposomes composed of Ch_{c}SₐPC and cholesterol at 2:1 molar ratio showed a similar leakage profile to that of Ch_{c}MₐPC. Since liposomes of Ch_{c}SₐPC/cholesterol (2:1) and DSPC/cholesterol (1:1) have the same ratio of cholesterol (50%) and chain length (C-18), their leakage profile difference may mainly come from the way cholesterol was incorporated in the bilayer.

A striking observation is that the Ch_{c}MₐPC liposomes remained intact while the contents of DSPC/cholesterol liposome were released gradually over the 4 week period tested. Liposome composed of Ch_{c}SₐPC and cholesterol at 2:1 molar ratio showed a similar leakage profile to that of Ch_{c}MₐPC. Since liposomes of Ch_{c}SₐPC/cholesterol (2:1) and DSPC/cholesterol (1:1) have the same ratio of cholesterol (50%) and chain length (C-18), their leakage profile difference may mainly come from the way cholesterol was incorporated in the bilayer. The covalent bonding in SML is strong enough to prevent the extraction of cholesterol from the bilayer by any form of non-covalent affinity from the serum proteins.

These results indicate that the compounds disclosed herein of general Formula I form more stable liposomes compared to conventional liposomes.

### Example 19

### Analysis of Cholesterol Exchange

Unilamellar liposomes were prepared by extrusion method. The donor liposomes consisted of 40% cholesterol (or mole equivalent from SML), 10% negatively charged 1,2-dipalmitoyl-sn-glycero-3-phosphatidylglycerol (DPPG), and 50% 1,2-dipalmitoyl-sn-glycero-3-phosphocholine (DPPC) (or the mole equivalent from SML). Specifically, the three donor liposomes were formulated at the following molar ratios: 1) PChcPC/DPPC/DPPG (50/40/10), 2) DChcPC/DPPC/DPPG (25/65/10), 3) Chol/DPPC/DPPG (40/50/10). Neutral 1-palmitoyl-2-oleoyl-sn-glycero-3-phosphocholine (POPC) liposome was used as the acceptor. After extrusion, the diameter of the donor liposomes was around 100 nm, and 140 nm for the acceptor liposome.

For the exchange experiments, 1 mL donor liposomes (10 mM) and 1 mL acceptor liposomes (100 mM, 10 fold) were warmed at 37° C. first, then mixed and incubated at 37° C. An aliquot (250 μL) of mixture was sampled at given time point and applied to a small (ca. 1 cm in length) anion exchange column (Q-Sepharose XL). The column was pretreated with 0.1 mL 10 mM POPC before the loading of the exchange sample to reduce the nonspecific binding of the neutral liposome. The column was eluted with 1 mL 10 mM NaCl, 10 mM HEPES pH 7.4 buffer. The eluate was lyophilized and analyzed by the cholesterol assay to quantify the amount of cholesterol exchanged.

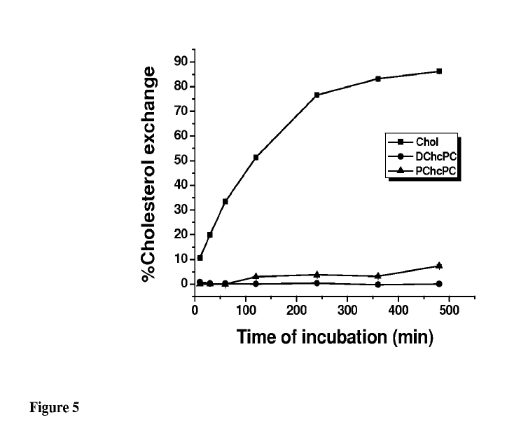

As shown in FIG. 5, liposomes containing DChcPC or PChcPC allowed for very little or no detectable exchange of cholesterol, particularly compared to control liposomes of Chol/DPPC/DPPG. These data illustrate that covalently linked cholesterol in SML compounds do not transfer out of the bilayer in significant amounts, while the free cholesterol in a conventional liposome has a half time of approximate 2 hour for cholesterol exchange.

Thus, the results of cholesterol exchange experiments further confirm that covalently linked cholesterol in compounds of general Formula I do not transfer out of the bilayer at significant or detectable levels, while the free cholesterol in a conventional liposome has a half time of approximate 2 hour for cholesterol exchange.

### Example 20

### Cytotoxicity Evaluation

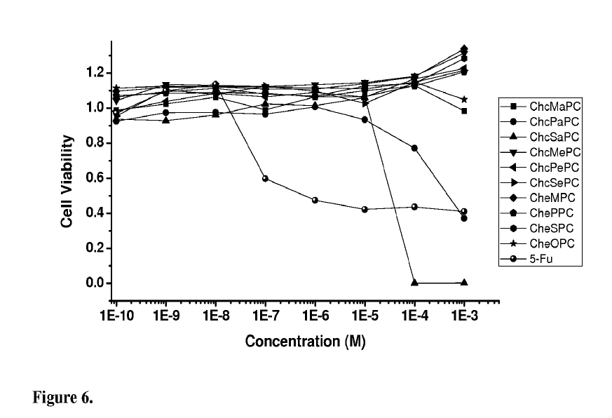

The cytotoxicity of the SML lipids was evaluated with the standard MTT (3-(4,5-dimethylthiazolyl-2)-2,5-diphenyltetrazolium bromide) assay method. Briefly, C26 cells were incubated with lipids of various concentrations for a period of time at 37° C. Then the medium was replaced with the MTT working reagents, and the cells were incubated for another two hours. The reagents were then carefully removed. The converted dye was solubilized in acidic isopropanol. The absorbance of the dye was measured at 570 nm with background subtraction at 650 nm. Cell viability data were then obtained by comparing the results of treated cells and untreated cells. The results of selected SML lipids were summarized in FIG. 6. Most SMLs showed no obvious cytotoxicity at concentration as high as 1 mM. Some SMLs containing the carbamate linkage demonstrated certain cytotoxicity such as ChcPaPC and ChcSaPC. Accordingly, it is possible to tune the toxicity of SMLs by varying the combination of R¹, R², and the linkage type of the compounds of the general formula I.

### Example 21

### Drug Release by Phospholipase A2

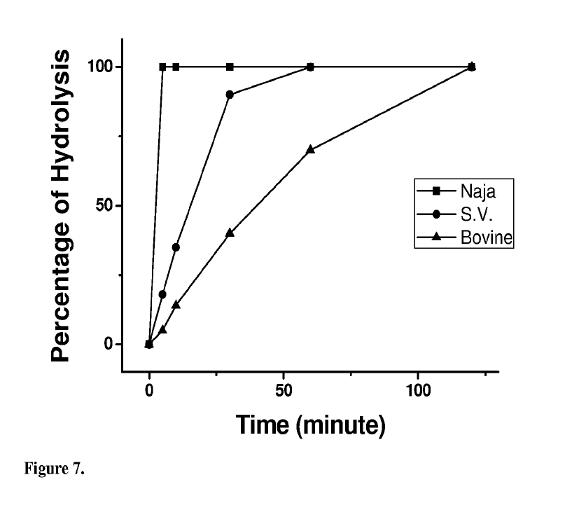

Retinoic acid was used as the model drug for the investigation of enzyme triggered drug release. The release of all-trans retinoic acid from SML7a (1-cholesteryl-2-all-trans-retinoyl-sn-glycerophosphocholine) was evaluated with phospholipase A2 (PLA2) from a variety of sources including bovine, naja mossambica, and streptomyces vialaceoruber. All PLA2s were from Aldrich-Sigma. A chloroform solution of SML7a (50 μmol) in 50 mL round-bottom flask was evaporated by the rotavapor. The flask was placed under high vacuum overnight. The lipid film was hydrated in 10 mL pH 8.8 buffer containing 20 mM Triton X-100, 10 mM CaCl₂, 10 mM HEPES, and 50 mM KCl. A clear solution was obtained after 10 mintures intermittent vortex at room temperature. Aliquots of the solution (250 μL) were preincubated at 37° C. for 10 minutes, then used for the PLA2 assay. Aliquots of PLA2 solution (25 units in 250 μL pH 8.8 buffer of 10 mM HEPES, and 50 mM KCl) were preincubated at 37° C. for 10 minutes, and mixed with the aliquots of pre-warmed solution of SML7a. At different time point, the hydrolysis samples were analyzed by TLC, and quantified by densimeter. The results were shown in FIG. 7. While all three PLA2 enzymes were able to completely release all-trans retinoic acid from SML7a, PLA2 from naja mossambica showed highest activity.

### Example 22

### Comparison of the Effect on Tumor Progression and Animal Survival of Doxorubicin Encapsulated in Liposomes of Various Compositions in the Balb/c Mouse Tumored with C26 Colon Carcinoma

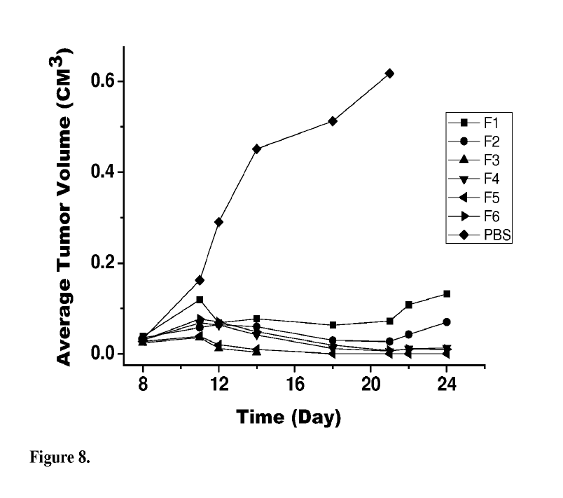

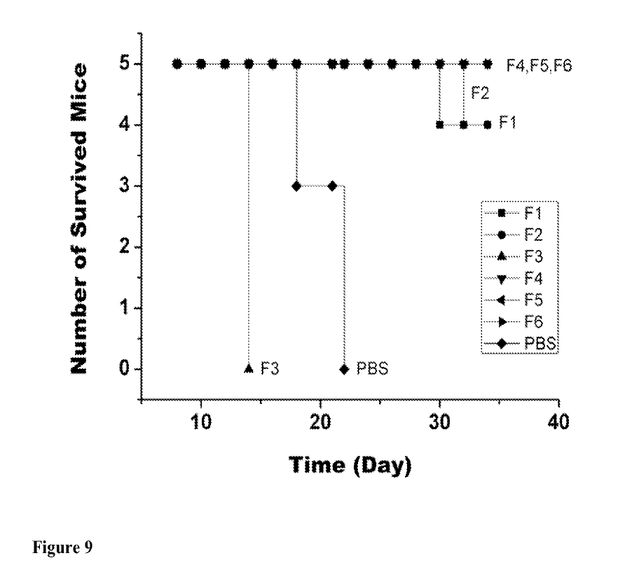

To demonstrate the general applicability of the SML-containing liposomes as drug carriers, an anticancer drug was encapsulated in a number of liposomes compositions that contained various steroylphospholipids. The effect of these formulations on tumor progression and animal survival were compared against the effect non-encapsulated doxorubicin or Doxil™, a commercial liposome formulation of doxorubicin that is approved by the Food and Drug Administration in the Balb/C mice tumored with C26 colon carcinoma. (FIGS. 8 and 9, Table 3).

In this model animals treated with the non-drug containing vehicle PBS had a median survival time of 22 days and no animals survived to 60 days (Table 3). Animals treated with the maximum tolerated dose of non-encapsulated doxorubicin (10 mg/kg body weight) had a median survival time of 26 days and no animals survived to 60 days. Animals treated with Doxil™ at 15 mg/kg had a median survival time greater than 60 days and 4 of 5 animals survived to day 60. Animals treated with doxorubicin in a di-sterol containing formulation DCHEMSPC-DSPC-PEGDSPE2000-αT, 33:61.8:5.0:0.2 at 15 mg/kg had a median survival time greater than 60 days and 5 of 5 animals survived to day 60 (Table 3). Doxorubicin encapsulated in various steroyl lipid containing formulations provided an outstanding therapeutic effect in the C26 colon carcinoma model as good or better than the currently approved lipid formulation that does not contain steroyl lipids (Table 3, FIGS. 8 and 9)

All synthetic phospholipids were purchased from Avanti Polar Lipid (Birmingham, Ala.) The SML phospholipids were synthesized as described above. Cholesterol (Chol), was purchased from Sigma Chemical Co. (St. Louis, Mo.). Dowex 50WX4 resin was purchased from Aldrich (Milwaukee, Wis.). Doxorubicin (DOX) was purchased from Bedford Laboratories (Bedford, Ohio). Culture medium (MEM Eagle's with Earle's BSS (EBSS)) was obtained from UCSF Cell Culture Facility. All other reagents were of analytical grade. Solutions were filtered through 0.2 micron sterile membranes into sterile containers. All solutions were sterile and pyrogen free.

Drug loaded liposomes of defined size were prepared by methods well known in the art and described in, for example, (Liposomes: 2nd edition, Oxford University Press, 2003, V. Torchilin and V. Weissig., Ed.). Lipid films were prepared by drying 10 μmoles of lipid mixtures dissolved in chloroform under a reduced pressure in glass tubes using a rotary evaporator at room temperature, followed by an overnight exposure to a high vacuum. Liposomes were prepared by rehydrating the thin lipid film above the transition temperature of the lipid in a sterile 250 mM ammonium sulfate solution in screw-capped glass tubes, followed by sonication in a bath type sonicator for 10 minutes at 60° C. The liposome preparation was then extruded through 0.1 micron polycarbonate membranes. Non-encapsulated ammonium sulfate was removed by dialysis against 100-fold volume of 5% glucose changed one time in a 24 hour time period at 4° C. Doxorubicin was encapsulated by incubating a solution of doxorubicin dissolved in 5% glucose for 2 hours at 60° C. with the ammonium sulfate containing liposomes. The non-encapsulated doxorubicin was removed from the liposomes by passing the preparation over a column containing Dowex 50WX4. The encapsulation efficiency was usually greater than 70%, with drug:phospholipid ratio of approximately 100 μg/umol total lipid. Mean vesicle diameters as measured by dynamic light scattering using the multimodal program ranged between 85-140 nm with a monodisperse particle size distribution (Malvern Instruments, UK). The liposome encapsulated doxorubicin preparation was filtered through sterile 0.2 micron membranes into sterile 15 mL sterile conical centrifuge tubes and stored at 4° C. until injected into animals.

All animal experiments were performed in compliance with the NIH guidelines for animal research under a protocol approved by the Committee on Animal Research at the University of California, San Francisco. For all chemotherapy experiments, on day 0, Balb/c mice were given subcutaneous injections of C26 tumor cells (4×10⁵ cells per mouse) in the right flank and were then randomized with 5 mice per group and numbered. Mice were weighed and tumor sizes were monitored daily during the experimental period. The tumor volume was estimated by measuring three orthogonal diameters (a, b, and c) with calipers; the volume was calculated as (a×b×c)×0.5 cm³. Tumors that were just palpable were defined as 1 mm×1 mm×1 mm. In each experiment the mice were monitored for up to 60 days post-inoculation or until one of the following conditions for euthanasia was met: 1) their body weight dropped below 15% of their initial mass; 2) their tumor was greater than 2.0 cm across in any dimension; 3) they became lethargic or sick and unable to feed; or 4) they were found dead. On day 60, all surviving mice were euthanized; however, if any of the surviving mice had palpable tumors on day 60, monitoring of all mice remaining in the experiment continued until day 90, at which point the mice were euthanized. All animals that survived 60 days also survived until day 90.

TABLE 3
Effect of Doxorubicin Delivered in Liposomes of Various Compositions
on Survival of Balb/C Mice Tumored with C26 Colon Carcinoma

| | Dose | | # of |
|---|---|---|---|
| | Con- | | Animals |
| | centration | Median | Surviving |
| | of Dox | Survival | at Day 60 |
| Formulation | (mg/kg) | Day | (n = 5) |
| Phosphate buffered saline | 0 | 22 | 0 |
| Free Dox | 10 | 26 | 0 |
| Doxil ™ | 15 | >60 | 4 |
| DCHEMSPC-DSPC-PEGDSPE-αT, | 15 | >60 | 5 |
| 33:61.8:5.0:0.2 | | | |
| PChcPC-PEGDSPE-αT, 94.8:5.0:0.2 | 15 | >60 | 4 |
| DCHEMSPC-PEGDSPE-αT, | 15 | >60 | 4 |
| 94.8:5.0:0.2 | | | |
| SeChc/PC/PEGDSPE/αT, 94.8:5.0:0.2 | 15 | >60 | 3 |
| SeChc/PC/PEGDSPE/αT, 94.8:5.0:0.2 | 10 | 26 | 2 |
| SeChc/PC/PEGDSPE/αT, 94.8:5.0:0.2 | 6 | 24 | 1 |
| SeChc/PC/PEGDSPE/αT, 94.8:5.0:0.2 | 2 | 24 | 0 |
| DChcPC/DSPC/PEGDSPE/αT, | 10 | >60 | 3 |
| 33:61.8:5.0:0.2 | | | |
| DChcPC/DSPC/PEGDSPE/αT, | 6 | 34 | 1 |
| 33:61.8:5.0:0.2 | | | |
| DChcPC/DSPC/PEGDSPE/αT, | 2 | 26 | 0 |
| 33:61.8:5.0:0.2 | | | |

Molar ratio of formulation components is indicated following each formulation.
DCHEMSPC = SML6b;
DSPC = distearoylphosphatidylcholine;
PEGDSPE = 1,2-distearoyl-sn-glycero-3-phosphoethanol-amine-N-[poly-(ethyleneglycol)-2000];
αT = α-tocopherol;
SeCHcPC = 1-Stearyl-2-cholesterylcarbonoyl-rac-glycero-3-phosphocholine.

### Example 23

### Amphotericin B Encapsulated Liposomes

Selected SMLs were evaluated for the encapsulation of amphotericin B. SML was formulated with the corresponding diacyl PC (same chain length) to encapsulate amphotericin B at various ratios. First, the chloroform solution of SML/diacyl PC lipids mixture at given ratio were evaporated, and lipid film was placed under high vacuum overnight. Given amount of amphotericin B DMSO solution (20 mg/mL) was then added to the lipid film followed by the addition of pH 7.4 PBS. The mixture was sonicated under argon at 60° C. for 1 hour. The mixture was then dialyzed against pH 7.4 PBS. The yellow solution obtained was sterilized by filtering through 220 nm membrane. Products were stored at 4° C. for future study. When amphotericin B was appropriately formulated with SML, for example SML:PC:AmB=2:2:1 (mole), most of the formulations were stable at 4° C. for more than one year such as formulations containing PeChcPC, MeChcPC, SChcPC and PChcPC. These results showed that optimized formulations could be achieved with SML for the encapsulation of amphotericin B.

### Example 24

### Liposomes for Protein Delivery

Certain therapeutic proteins can bind to the liposome surface. This can lead to stabilize of the protein and a longer circulation time when the liposome protein complex is injected into animals. For example, recombinant FVIII binds non-covalently but with high affinity to the external liposome surface. Factor VIII when reconstituted with synthetic PEGylated liposomes, composed of 90% (wt/wt) palmitoyloleoyl-phosphatidylcholine (POPC) and 1,2-distearoyl-sn-glycero-3-phosphoethanol-amine-N[poly-(ethyleneglycol)-2000] (DSPE-PEG 2000), 97:3 molar ratio, suspended in 50-mM sodium citrate buffer (9% wt/vol solution) have been used to prolong the circulation time of Factor VIII and decreases bleeding in preclinical models and humans. This formulation is not optimal because in the absence of cholesterol it is eliminated from circulation too quickly.

Stabilized SML liposome formulations that illustrate delivery of proteins are as follows. An SML liposome formulation composed of SML3d synthesized as described in example 3 and DSPE-PEG 2000 (Avanti Polar Lipids) in a 97:3 molar ratio, respectively) is prepared as described in the doxorubicin example, except that 100 μmoles of the lipid mixture is resuspended in 50 mM sodium citrate pH 7.0. One mL of the unilamellar SML3d-DSPE-PEG2000 liposome is mixed with 100 IU units of recombinant Factor VIII, Kogenate FS (Bayer HealthCare Pharmaceuticals, Berkeley). The SML3d-DSPE-PEG2000 liposome formulation provides a more stable liposome for formulating Factor VIII than does a formulation lacking cholesterol such as palmitoyloleoyl-phosphatidylcholine (POPC) and 1,2-distearoyl-sn-glycero-3-phosphoethanol-amine-N[poly-(ethyleneglycol)-2000] (DSPE-PEG 2000), 97:3 molar ratio. The SML3d-DSPE-PEG2000 liposome can be used to improve the in vivo activity of Factor VIII.

Proteins may form stable particles with SML liposomes without loosing activity. Proteins with polyhistine tag are anchored on SML liposomes through lipid-tri-nitrilotriacetic acid such as DOD-tri-NTA described in Bioconj. Chem. 2006, 17, 1592-1600. A typical formulation includes 5% DOD-tri-NTA, 50% diacyl phosphocholine, and 45% m-SML. The protein is incorporated on the liposome through the NTA-Ni-Histidine interaction. The protein loaded liposome is purified by passing size exclusion column. The formulation stability and the protein activity is then evaluated with appropriate methods.

### Example 25

### The Use of SMLs for Hypercholesterolemia

Phytosterols such as β-sitosterol and β-stigmasterol are known to inhibit cholesterol absorption and to reduce the plasma cholesterol level in humans. SMLs containing β-sitosterolare useful for the treatment of hypercholesterolemia.

SMLs containing β-sitosterol are formulated as food additives, or as an injection to help lower the blood cholesterol level. For example, the SML disterol lipid SML6d is dissolved in tert-butanol at a concentration of 30 mg/mL then sterilized by filtration through a 0.1 micron glass filter. The sterile lipid solution is frozen at −70° C. then lyophilized for 24 h to complete dryness in a lyophilizer. The dry lipid powder (150 mg) is mixed with 30 mg pectin, 42 mg calcium (as di-calcium phosphate), 26 mg phosphorous (as di-calcium phosphate) and microcrystalline cellulose. The dry powder is filled into a gelatin capsule to provide a dose of 150 mg of the disitosterolphosphatidylcholine. When taken orally just prior to a meal this sitosterol derivative can be used to inhibit cholesterol absorption.

### Example 26

### Preparation of Micro-Bubbles Using SMLs

Microbubbles are gas filled bubbles that are stabilized by a monolayer of lipid. In the past microbubbles have been prepared with synthetic phospholipids mixtures lacking cholesterol. This is because cholesterol rapidly leaves the monolayer when the microbubbles are placed in contact with biological membranes and lipoproteins: this results in destabilization of the microbubble.

SMLs can be used in the formulation of microbubbles. Liposomes prepared from the SML have an increased retention of contents in the presence of serum (Example 18). Microbubbles are prepared from decafluorobutane gas and stabilized with a monolayer composed of a mixture of SML4a (Example 4) and PEG-DSPE-2000 (Avanti Polar Lipids, Alabaster, Ala.) in a molar ratio of 90:10. An appropriate amount of SML4a, PEG-DSPE-2000, (90:10, molar ratio) in chloroform is added to a glass test tube. Chloroform is removed under N₂ followed by evaporation under a vacuum for at least 2 h. A buffer diluent consisting of 100 mM Tris (pH 7.4): glycerol:propyleneglycol (80:10:10, volume ratio) is added to the dried lipids to create a lipid concentration of 1 mM (1 mg/mL). The lipid suspension is mixed well above the phase transition temperature of the lipids (60° C.) to form a milky solution of multilamellar vesicles. The suspension is sonicated to clarity using a bath sonicator (20 kHz, 100 W, 10 min). The liposome solution at a final concentration of 1 mg/mL is aliquoted in 1 mL lots to a 2-mL vial. Then, 10 cm³ of decafluorobutane gas (Flura, Newport, Tenn., USA) is slowly injected into the vial through the rubber cap and air is exchanged using a needle (20G1, short Bevel, Becton-Dickinson) as a vent. The vial is immediately capped using an aluminum seal on the rubber cap. The sealed vial containing the liposome solution with the decafluorobutane headspace can be stored at 4° C. until use. Microbubbles are formed via mechanical agitation of the vials of liposome solution using a Biobead shaker. Upon shaking the vial for 45 s, the solution becomes milky, and can be drawn into a 3-mL syringe and diluted to a final volume of 3 mL using 10 mM phosphate buffered saline (PBS, pH 7.4). The liposomes (unincorporated into microbubbles) and submicron-sized bubbles can be removed from the solution by flotation at 300×g for 3 min.

Microbubbles formed from a composition containing compound SML4a can then be injected for imaging or for the delivery of molecules using ultrasonic disruption at defined sites in an animal. The availability of SMLs with a range of physical chemical properties permit the formulation of microbubbles with precisely controlled properties.

### Example 27

### Diagnostic Assay Platform Using Polydiacetylene Vesicles or Monolayers Prepared from Compound SML9a

Liposome based colorimetric assays have in which a polydiacetylene is formed in a bilayer vesicle have been proposed for a point of use diagnostic assay because of a large shift in the absorption spectrum of the polydiacetylene material upon the binding of an analyte to a receptor incorporated into the surface of the polymerized vesicle. The sensitivity of this assay depends upon the length of the polydiacetylenic polymer and its orientation. Compositions that are usually used for this application consist of synthetic phospholipid such as dimyristoylphosphatidylcholine (DMPC) mixed with 10,12 tricosadiynoic acid (TRCDA). Stock solutions of TRCDA and DMPC are prepared in methylene chloride at a 6/4 molar ratio. Liposome prepared from this composition are subsequently polymerized under a hand-held UV lamp at 254 nm. These compositions are not suitable for use in many biological fluids because proteins from the fluid interact with the liposome surface and bring about a non-specific change in color. Attempts to include free cholesterol into these mixtures to reduce protein absorption have not been satisfactory because upon polymerization of the TRCDA, the cholesterol phase separates and the ability of the liposome to respond to the analyte is compromised. The DMPC in the above composition can be replaced with SML2c to provide a more stable TRCDA composition.

Another approach is to use SML compound SML9a described in example 9 to enhance stability. SML9a contains the polymerizable tricosadiynoic acid attached to a cholesterol containing phosphatidylcholine head group. In the liposome bilayer, the TRCDA is favorable oriented by the adjacent cholesterol to undergo facile polymerization. When polymerized the cholesterol is unable to phase separate because it is attached to the phospholipids. The SML9a lipid is dried onto the sides of a glass tube along with a lipid-linked receptor such as ganglioside GM1 from a chloroform solution in a 97/3 mole ratio and the solvent evaporated. Phosphate buffered saline is added to the dried lipid mixture (final concentration, 1 mM total lipid) and bath sonication is performed. The sample is heated to 45° C. during sonication in order to ensure that the lipids are above the main phase-transition temperature. Because of the presence of covalently attached cholesterol in the SML, production of the liposomes can be done at a lower temperature; this is important for the stability of many biological targets (e.g., receptors) that may be included in the assay.

The SML liposome preparation is filtered warm through a 0.8 μM polycarbonate membrane, and stored at 4° C. overnight. The sample is brought to room temperature and polymerized using 254 nm light hand held UV lamp to yield a dark blue/gray solution of the polymerized vesicles. This polymerized monosterol SML liposome composition can be used to detect the presence of E. coli entertoxin in biological fluid because the enterotoxin binds to the GM1. The covalently attached cholesterol moiety in the SML stabilizes the polymerized liposome from insertion of proteins found in the biophase and does not transfer from the liposome into biomembranes in the biophase as free cholesterol will. Thus the SML facilitates a more sensitive and more stable point of use diagnostic system.

### Example 28

### Use of Monosterol SMLs for a Rejuvenating Hair Conditioner

Phospholipids and cholesterol are essential components of the human body and typical ingredients for personal care products that nourish, moisturize, clean and condition skin and hair. The mono and disterol SML glycerolipids and SML sphingolipids combine these two important components in a single molecule. They also provide them in either biodegradable or biostable versions.

In this example, a hair conditioning composition is made from the following ingredients all added at a weight percent: water 86.6%, hydroxyethyl cellulose 0.7%, glycerol distearate 0.7%, cetyl alcohol 2.0%, the SML compound SML4d (example 4) 10%. To prepare this mixture, the hydroxyethylcellulose is added to the water under high speed constant stirring conditions and heated to 60° C. The remaining ingredients are added with continued stirring and the temperature is increased to 70° C. Coloring agents, fragrances and antimicrobial agents can be added at this point. The mixture is agitated until the components form a smooth fluid with a pleasing consistency. The composition is cooled to room temperature. This hair conditioner is applied to the hair to give it a healthy and pleasing appearance. The ether linked monosterol SML used in this application provides a long shelf life and excellent mixing for the various components in the formulation.

### Example 29

### Nanoemulsions Formed Using Monosterol and Disterol SMLs

Nanoemulsions or sub-micron emulsions are oil-in-water emulsions with mean droplet diameters ranging from 50 to 1000 nm. Usually, the average droplet size is between 100 and 500 nm. Usually, nanoemusions contain 10 to 20 percent oil stabilized with 0.5 to 2 percent egg or soybean lecithin. The SMLs described in this example are ideal emulsifiers that will not permit the sterol component to phase separate from the amphipathic head group as free cholesterol can do.

The preparation of SML nanoemulsions in this example employs high-pressure homogenization and SML compound SML5d. The particles which are formed exhibit a liquid, lipophilic core separated from the surrounding aqueous phase by a monomolecular layer of phospholipids. The structure of such lecithin stabilized oil droplets can be compared to chylomicrons. Nanoemulsions therefore differ from the liposomes, where a phospholipid bilayer separates an aqueous core from a hydrophilic external phase. Alternatively, nanoemulsions prepared with an excess of phospholipids may concurrently form liposomes.

A nanoemulsion for skin care purposes can be formulated with biodegradable SMLs. For example, SML5d generates cholesterol and oleic acid as the SML is hydrolyzed over tithe, and thus can be used in skin care products. Table 4 below illustrates an SML nanoemulsion skin care composition.

TABLE 4
nanoemulsion

| Ingredient | Function | Weight (%) |
|---|---|---|
| Evening Primrose Oil | oil | 25.0 |
| Tocopherol | Antioxidant | 5.0 |
| SML5d (example 5) | Emulsifying Agent | 4.0 |
| Water | Diluent | 67.0 |

A variation of the above skin care formulation is one in which 1 weight percent of the SML5d lipid (Example 5) in the above formulation (Table 4) is replaced by 1% of SML7a (Example 7). The SML7a lipid provides a skin care composition having a sustained release form of trans-retinoic acid. This later skin care formulation can be used to rejuvenate the skin and remove wrinkles.

### Example 30

### Encapsulation and Delivery of Two or More Drugs from the Same Liposome

The use of drug combinations is a widely adopted strategy in clinical cancer therapy. Although drug interaction at different drug ratios can be systematically studied in vitro, these ratios cannot be easily translated in vivo due to differential pharmacokinetic characteristics of different drugs. Coencapsulation of two drugs into liposomes can “synchronize” the distribution of the drugs if the drugs can be stably entrapped inside. This theoretically would allow for a more direct translation of in vitro results to in vivo. However, given the poor stability and leakage of material from standard liposomes, the encapsulated drugs can be released at different rates, making it difficult to predict effective free drug concentrations.

In this example, stabilized SML liposomes are exploited for controlled, synchronized release of various drug combinations. As demonstrated above, there is no difference in osmotic induced release of a model compound between a DMPC/free cholesterol composition and a liposome prepared from the SML-based phospholipid SML1c (ChcMaPC) under in vitro conditions. However, under conditions that mimic the in vitro condition, there is a substantial difference in release of calcein in the presence of 30% fetal bovine serum from the DMPC/free cholesterol composition ˜80% leakage by 7 days, as compared to less than 1% leakage in 28 days from the SML-based SML1c liposome (FIG. 4). Thus, co-encapsulation of two drugs in liposomes composed of SMLs may greatly reduce the difference between in vitro release and in vivo release, including a better prediction of in vivo release from in vitro data.

Stable coencapsulation of the following two drug combinations synchronizes their delivery in vivo from liposomes prepared from the SMLs: irinotecan/fluoxuridine, daunorubicin/cytarabine, cisplatin/daunorubicin, cisplatin/doxorubicin, vinorelbine/cisplatin, protein kinase inhibitors/doxorubicin, mithramycin/nitrogen mustard, paclitaxel/topotecan, 7-hydroxystaurosporine/camptothecins, leucovorin/5-fluorouracil, leucovorin/fluoroorotic acid, mercaptopurine/cytosine arabinoside, vinorelbine/paclitaxel, vinorelbine/doxorubicin, cytosine arabinoside/cisplatin, reversatrol/cytosine arabinoside, carboplatin/gemcitobine, topotecan/cisplatin or from combination of drug with siRNA or other oligonucleotides. The drugs are co-encapsulated in accordance with their known effective dosages in vivo, and their release profiles determined in vitro, by separating the encapsulated drug from the released drug on a size exclusion column.

### Example 31

### Liposomes Composed of SMLs for Pulmonary Drug Delivery

Pulmonary drug delivery systems have been used for decades to deliver drugs for the treatment of respiratory disorders such as asthma, emphysema, gram negative bacterial infections, and fungal infections. The delivery of aminoglycosides such as tobramycin, to patients suffering from cystic fibrosis has become a mainstay of antibacterial treatment in CF patients. Advancing technologies are overcoming the challenges of phagocytosis, particle size optimization, and degradation and are enabling utilizing the huge surface area of the lung to deliver drugs into the blood circulation. Lungs are considered the best alternative for drugs such as proteins like insulin needing to bypass the gastrointestinal tract.

In spite of this great need, there are no approved liposome encapsulated drugs for delivery into the lung. The reason for the absence of the use of liposome for pulmonary drug delivery is that the lung is filled with surfactant which can disrupt liposomes made from currently used synthetic lipids. Free cholesterol cannot be used to stabilize current liposomes because free cholesterol rapidly exchanges from the liposome into the lung surfactant.

The SML compounds described here avoid the problem of liposome disruption by lung surfactant, while also facilitating the controlled release drugs out of the liposome into the lung.

In this example, SML liposomes containing amphotericin B are prepared from the mixture of a SML and diacylphospholipds described in example 23, and by methods well known in the art and described in, for example, (Liposomes: 2nd edition, Oxford University Press, 2003, V. Torchilin and V. Weissig., Ed.). The amphotericin B loaded SML liposomes are aerosolized into the lung of a test animal or patient as a treatment for fungal infections such as aspergillosis. In another example, amphotericin B loaded SML liposomes prepared according to the formulation described in example 23 are lyophilyzed or freeze dried, and delivered into the lung as a dry powder. This is particularly advantageous because it represents a more convenient dosage form for the patient, and a more stable dosage form.

In another example, the disterol SML lipids described in example 6 are used to prepare antibiotic loaded liposomes that are stable in the presence of lung surfactant. Antibiotics such as tobramycin or ciprofloxacin can be used for this purpose. The liposomes are prepared from the pure SML6b or from a combination of SML6b and various synthetic diacylphospholipids such as distearoylphosphatidylcholine (DSPC), dipalmitoylphosphatidylcholine (DPPC), dimyristoylphosphatidylcholine (DMPC) or distearoylphosphatidylethanolamine-PEG2000 (PEG-DSPE-2000). Liposomes of defined size are prepared by methods well known in the art and described in, for example, (Liposomes: 2nd edition, Oxford University Press, 2003, V. Torchilin and V. Weissig., Ed.). Lipid films are prepared by drying 100 μmoles of lipid mixtures dissolved in chloroform under a reduced pressure in glass tubes using a rotary evaporator at room temperature, followed by an overnight exposure to a high vacuum. Specific compositions are 95 μmoles SML6b and 5 μmoles DSPE-PEG or 40 μmoles SML6b, 55 μmoles DSPC and 5 μmoles PEG-DSPE-2000 or SML2a and 5 moles PEG-DSPE-2000. Liposomes are prepared by rehydrating the thin lipid film above the transition temperature of the lipid in a sterile 200 mg/mL solution of tobramycin in screw-capped glass tubes, followed by sonication in a bath type sonicator for 10 minutes at 60° C. The liposome preparation is then extruded through 0.1 micron polycarbonate membranes. Non-encapsulated tobramycin is removed by dialysis against 100-fold volume of 50 mM tris/HCL, pH 7.4 changed one time in a 24 hour time period at 4° C. The encapsulation efficiency is usually greater than 10%, with drug:phospholipid ratio of approximately 200 μg/mol total lipid. Mean vesicle diameters as measured by dynamic light scattering will range between 100 nm to 150 nm depending on which formulation is selected. (Malvern Instruments, UK). The liposome encapsulated tobramycin preparation is filtered through sterile 0.2 micron membranes into sterile 15 mL sterile conical centrifuge tubes and stored at 4° C. until aerosolized into test animals. A pharmaceutically acceptable formulation of this composition can be aerosolized into patients.

In yet another example, cationic lipid formulations prepared from the carnitine based SML compounds (Example 11) are used to form complexes with anionic oligonucleotides such as siRNA or polynucleotides such as DNA. Lipid films are prepared by drying 100 μmoles of lipid mixtures dissolved in chloroform under a reduced pressure in glass tubes using a rotary evaporator at room temperature, followed by an overnight exposure to a high vacuum. Specific compositions are 30 μmoles SML6a and 70 μmoles 1,2-Dioleoyl-3-Trimethylammonium-Propane (DOTAP). Liposomes are prepared by rehydrating the thin lipid film above the transition temperature of the lipid in a sterile 10 mM tris/HCl pH 7.0 in screw-capped glass tubes, followed by sonication in a bath type sonicator for 10 minutes at 25° C. The liposome preparation is then extruded through 0.1 micron polycarbonate membranes. The nucleic acid to be delivered is mixed with the liposome preparation at a 3/1 mole ratio of trimethylammoniun groups to nucleic acid phosphate groups. Under these conditions all of the nucleic acid is associated with the lipid particle. Mean particle diameters as measured by dynamic light scattering will range between 100 nm to 200 nm depending on which formulation is selected. (Malvern Instruments, UK). The liposome associated nucleic acid preparation is filtered through sterile 0.4 micron membranes into sterile 15 mL sterile conical centrifuge tubes and stored at 4° C. until aerosolized into animals. This formulation or a pharmaceutically acceptable formulation thereof is suitable for transferring polynucleotides or siRNA into the lungs of test animals or patients.

### Example 32

### Liposomes and Lipid Particles Composed of SMLs and Mixtures of SML and Non-SML Lipids for Antigen Delivery for the Induction of Vaccines

SML compounds have exceptional stability in biological fluid while maintaining a high degree of fluidity. These characteristics make them good candidates to deliver antigens.

In one example, a SML-based formulation for vaccine applications is prepared from SML8a (example 8) and 1,2-Dioleoyl-3-Trimethylammonium-Propane (DOTAP) mixed at a mole ratio of 1/1. In another example, the cationic SML based on carnitine (SML11a) is substituted in place of DOTAP. Lipid films are prepared by drying 100 mmoles of lipid mixtures dissolved in chloroform under a reduced pressure in glass tubes using a rotary evaporator at room temperature, followed by an overnight exposure to a high vacuum. Liposomes are prepared by rehydrating the thin lipid film above the transition temperature of the lipid in a sterile 10 mM tris/HCl pH 7.0 in screw-capped glass tubes, followed by sonication in a bath type sonicator for 10 minutes at 25° C. The liposome preparation is then extruded through 0.1 micron polycarbonate membranes. The protein or peptide epitope, known as the antigen, is mixed with the liposome preparation at a various weight ratios from 30/1 lipid/antigen to 1/1 lipid to antigen. If the antigen has a net negative charge (dependent on the isoelectric point of the antigen), the antigen associates with a lipid particle comprising a cationic SML formulation. Mean particle diameters as measured by dynamic light scattering will range between 100 nm to 300 nm depending on which formulation is selected. (Malvern Instruments, UK). The SML liposome associated antigen preparation is filtered through sterile 0.4 micron membranes into sterile 15 mL sterile conical centrifuge tubes and stored at 4° C. until administered into test animals. The formulation is administered by intradermal, intramuscular, subcutaneous, intranasal, oral, pulmonary or parenteral routes at a dose suitable for the route of administration and species of animal. A pharmaceutically acceptable formulation of this composition can be administered to patients.

In another example, an anionic SML liposome formulation is prepared using the SML compound SML8a mixed with distearoylphosphatidylglycerol at a 9 to 1 mole ratio. In addition, monophosphoryl lipid A (Sigma, St. Louis, Mo.) at a ratio of 25 μg to 1 mg of lipid is prepared as described above. To increase the immune response from this anionic SML liposome formulation, the peptide or protein antigen is attached to the liposome bilayer at a ratio of 25 to 100 microgram antigen per mg of lipid. The attachment can be either covalently or by non-covalent means, such as through charge interactions or using metal chelation interactions between a chelated metal on the liposome surface and a histidine tag (“His-Tag”) on the antigen. The formulation is administered into an animal by intradermal, intramuscular, subcutaneous, intranasal, oral, pulmonary or parenteral routes at a dose suitable for the route of administration and species of animal. A pharmaceutically acceptable formulation of this composition can be administered to patients.

In an additional example, a solid core SML-based lipid emulsion is prepared for antigen delivery. Solid core SML particles are prepared from triglycerides containing stearoyl or palmitoyl chains at 25° C. A suitable SML-based vaccine formulation is prepared from SML8a (example 8) and 1,2-Dioleoyl-3-Trimethylammonium-Propane (DOTAP) mixed at a mole ratio of 1/1 as described above.

### Example 33

### Lipoplexes Containing SMLs for Nucleic Acid Delivery

Cationic lipid systems can be used for delivery of nucleic acids such as RNA, DNA, oligonucleotides, siRNA or other oligo and polynucleotides into cells in culture and cells in animals or patients. There are a very large number of cationic lipids and systems containing cationic lipids that have been described in the literature. All of these have been limited in their in vivo nucleic acid transfer efficiency because the particles are not stable in vivo. For instance, the lack of stability can be due to the fact that cationic systems devoid of cholesterol quickly become covered with proteins when injected into animals or patients. Those that contain free cholesterol for stabilizing the lipid system lose the free cholesterol into cell membranes when administered in vivo. Lipoplex compositions containing SML compounds avoid this problem.

Cationic SML-based liposomes or cationic SML-based micelles prepared as described above are admixed with an anionic nucleic acid to form a complex via charge interactions between the negative charge on the nucleic acid and the positive charge on the cationic SML-based lipid particle. These complexes are known as lipoplexes or SML lipoplexes. When DNA is the polynucleotide complexed with the cationic SML-based liposome or cationic SML-based micelles, the SML formulation can be used to deliver DNA (e.g., for “gene transfer”). When an oligonucletides such as siRNA is complexed with a cationic SML-based liposome or cationic SML-based micelle, the SML formulation can be used to deliver the siRNA or modified siRNA (e.g., for “gene silencing”).

In one example, a lipid mixture consisting of a disterol SML such as compound SML6a is mixed with 1,2-Dioleoyl-3-Trimethylammonium-Propane (DOTAP) at a mole ratio of SML/DOTAP in chloroform solution. Lipid films are prepared by drying 100 μmoles of the lipid mixtures dissolved in chloroform under a reduced pressure in glass tubes using a rotary evaporator at room temperature, followed by an overnight exposure to a high vacuum. Liposomes are prepared by rehydrating the thin lipid film above the transition temperature of the lipid in a sterile 10 mM tris/HCl pH 7.0 in screw-capped glass tubes, followed by sonication in a bath type sonicator for 10 minutes at 25° C. The liposome preparation is then extruded through 0.2 micron polycarbonate membrane. The nucleic acid dissolved in 10 mM tris/HCl pH 8.0 is then added to the cationic SML liposome so that the cationic lipid positive charge to nucleic acid negative is 3 to 1, which is designated as a SML lipoplex. The SML lipoplex is then be added to cells in culture or administered to a test animal by various routes, such as by injection. A pharmaceutically acceptable formulation of this composition can be administered to patients.

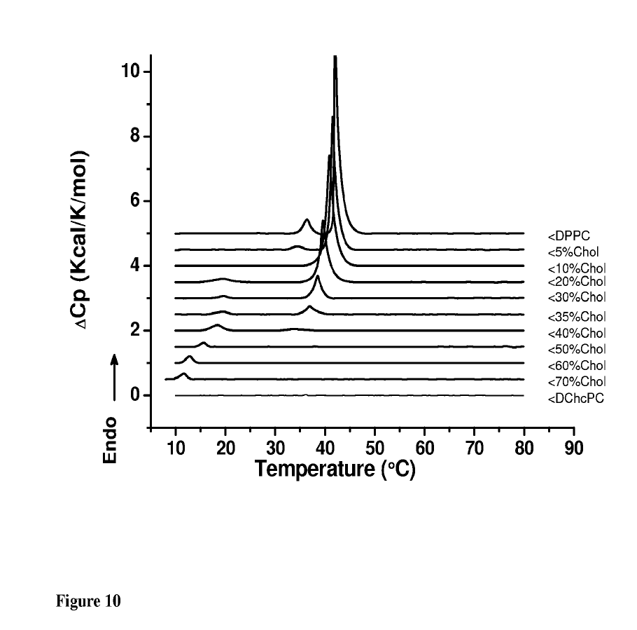

Lipid SML6a provides cholesterol to the lipoplex in a form that does not readily transfer through the aqueous phase into biological membranes. So the lipoplex is much more stable in the biophase than one formed with free cholesterol. Moreover, lipid SML6a can induce the formation of a new phase as the cholesterol to lipid ratio increases above about 30 mole percent cholesterol. This is evident in the DSC trace in FIG. 10, where a new transition is observed around 20° C. when the equivalent free cholesterol mole percent is at 30 mole percent. Lipoplexes prepared from this lipid mixture are good polynucleotide transfer reagents. The cationic lipid in this example is DOTAP, but any cationic lipid monocationic or multicationic can be used in place of DOTAP.

In another example, the carnitine based SML compound (example 11) is used as a single species to form a cationic SML particle and interact with a nucleic acid such as siRNA. The properties of such cationic SML particles are adjusted based on the position of attachment of the sterol moiety and the aliphatic moiety as well as the aliphatic chain length, degree of unsaturation or branching of the aliphatic chain. The complex between the cationic SML-based liposome and the nucleic is formed by adding the nucleic acid in 10 mM tris/HCl pH 8.0 to the preformed cationic particle in 10 mM tris/HCl pH 7.0 at a 3/1 cationic to anionic charge ratio. The SML lipoplexes so formed are efficient nucleic acid delivery vehicles both in cell culture and in vivo. An added advantage of SML-based lipoplexes formed from a single SML compound and a nucleic acid is that they are readily lyophilized and can be quickly rehydrated into nucleic acid transfer-active lipoplexes.

Thus, as illustrated in this example, a wide variety of univalent and multivalent SML compounds can be readily formed as lipoplex compositions for use as nucleic acid delivery vehicles.

### Example 34

### Nanolipoparticle Compositions Containing Reducible SMLs for Nucleic Acid Drug Delivery

To obtain long circulating nanoparticles that are capable of delivering nucleic acids to a defined target in vivo, it is necessary to remove or effectively shield the cationic charges on the nanoparticle surface. This has been attempted in the past by using a titratable cationic group that is not protonated at pH 7.4, by removing the cationic group either by reacting it to form an anionic or neutral species after the nanoparticle is formed or by to exchange the cationic group with an anionic or neutral species in a disulfide exchange reaction. Another approach is to avoid the use of the cationic species and replace the electrostatic interaction with a hydrogen bonding species that is not cationic at pH 7.4. In spite of these efforts, stability of the nanolipid particles is compromised by the lack of stability in vivo, and in particular, from the disruptive interactions with plasma proteins and lipoproteins found in the blood and other body fluids. The SML compounds described herein can be used to increase the stability of nanolipoparticles by providing a non-exchangeable sterol, as well as remove or effectively shield the cationic charges on the nanoparticle surface.

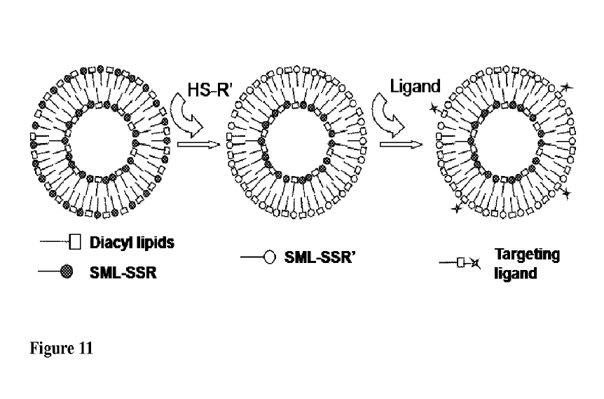

In one example, an SML nanoparticle is prepared that exploits a disulfide bond as cleavable linkage for bioresponsive polynucleotide delivery in vivo. Cleavage of the linker in vivo relies on the high redox potential difference between the oxidizing extracellular space and the reducing intracellular milieu. For instance, cationic SML nanoparticles containing disulfide-linked, cationic functionalized lipids are stable in the extracellular matrix but cleaved from the lipid anchor in the reductive milieu in the cytoplasm. Cleavage of the cationic moiety from the lipid releases the charge-condensed DNA from the nanoparticle, so that the DNA migrates into the nucleus. In this example, a dialysis method with the cationic disulfide containing SML compound SML13i (Example 13) is used to encapsulate plasmid DNA into a PEG-shielded nanolipid particle (FIG. 11). The positive surface charge is then converted into either a neutral or negative charge by the disulfide exchange reaction with cysteine (Cys) and glutathione (GSH), respectively. Thus this method can be used to create a particle with either a neutral, zwitterionic or non-ionic surface. In addition, now the non-cationic SML nanoparticle is stabilized by the SML. The non-cationic nanolipid particle can be further modified by the incorporation of a targeting ligand such as an antibody reactive against a cell surface molecule to bind the nanoparticle to the cell surface.

In a specific example, plasmid DNA is added into a lipid mixture of PEG-lipid/SML5d/cationic disulfide SML13i mole ratio 1/5/5 in 28 mM octylglucoside and the octylglucoside is removed by dialysis against 20 mM Hepes pH 8.5. Total dried lipids 2.5 μmol (molar ratio of PEG-lipid/SML5d/SML13i mole ratio 1/5/5) are hydrated in 1.47 ml of 28 mM n-octyl-β-D-glucopyranoside (OG) in 5 mM trishydroxymethyl aminomethane (Tris) buffer (pH 8.5) for 0.5 h. DNA plasmid (137.3 μg) in the same volume of detergent buffer is added into the lipid solution with gentle vortex for 30 seconds. The solution is then transferred to a Slide-A-Lyzer™ dialysis cassette (MWCO 10 K, Pierce, Rockford, Ill.) and dialyzed against 1 liter of 20 mM HEPES buffer (pH 7.4) at 4° C. for 2 days with four changes of the dialysis buffer.

To modify the surface charge of the particles, GSH or Cys, with a molar ratio 10 times greater than the amount of SML13i used for the particle formation, is added into the SML nanolipid particle solution, and the solution is incubated for 5 min, then placed in a dialysis cassette and immediately dialyzed against 1 liter of 20 mM HEPES buffer (pH 7.4) at 4° C. to remove the reducing reagent.

The particle diameter of the resulting SML nanolipid particle is substantially smaller (ca. 100 nm diameter) compared to the lipoplex formulation (170˜360 nm) at the same charge ratio. The particle surfaces can be further modified by mixing the SML nanolipid particle with excess reducing reagents: GSH and Cys, respectively for a short period. The unreacted reducing reagent is removed from the SML nanolipid mixture by dialysis. The particle diameter of the resulting SML nanolipid particles usually increases by about 40 nm. About 50% of the encapsulated DNA plasmid remains within the particles after surface modification as compared to the 86% encapsulation value in a cationic lipoplex. The surface charge of the cationic SML nanolipid particle treated with GSH or with Cys is converted to an anionic or a neutral charge, respectively. This alteration of surface charge is due to the exchange of the cationic headgroup with either the negatively charged GSH or the zwitterionic Cys. These SML nanolipid particles are also suitable for in vivo polynucleotide or siRNA delivery and can be modified further by the incorporation of a lipid-linked targeting ligand into the lipid mixture used to prepare the targeted SML nanolipid particle.

### Example 35

### Preparation of Lipids SML15a-j

A particular synthetic scheme for the synthesis of lipids SML15a, SML15b, SML15c, SML15d, SML15e, SML15f, SML15g, SML15h, SML15i and SML15j (referred to collectively as SML15a-j) is outlined in Scheme 15. This scheme is exemplified below by the detailed description of the synthesis of lipids SML15f.

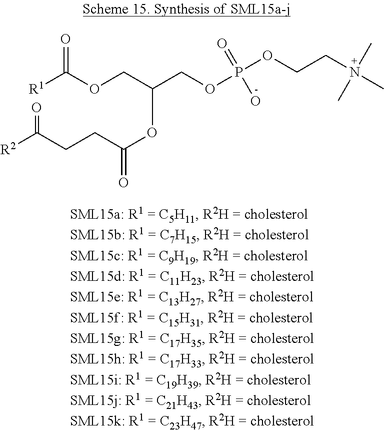

1-Palmitoyl-2-cholesterylhemisuccinoyl-sn-glycero-3-phosphocholine (SML15f, PChemsPC): To a solution of 1-palmitoyl-2-hydroxy-sn-glycero-phosphocholine (0.95 g, 1.91 mmol) and cholesterylhemisuccinate (1.86 g, 3.82 mmol) in ethanol-free dry chloroform (50 mL) at room temperature, were added DMAP (0.24 g, 1.91 mmol) and DCC (0.79 g, 3.82 mmol). The reaction mixture was stirred at r.t. for 24 h. The mixture was filtered and the filtrate was concentrated by rotary evaporation. The residue was applied to HPFC for purification (CHCl₃ to CHCl₃-MeOH—H₂O 65/25/4). TLC: R_{f}=0.54 (eluent C). ¹H NMR (CDCl₃), δ0.68 (s, 3H); 0.85-1.65 (m, 62H); 1.84-2.05 (m, 5H); 2.29-2.31 (m, 4H); 2.55-2.62 (m, 4H); 3.31 (s, 9H); 3.78 (m, 2H); 4.04 (m, 2H); 4.24 (m, 1H); 4.41 (m, 3H); 4.55 (m, 1H); 5.21 (m, 1H); 5.40 (d, J=4.4, 1H). MALDI-MS calcd for C₅₅H₉₉NO₁₀P⁺ [M+H]⁺ 964.71. Found 964.68.

### Example 36

### Preparation of Lipids SML16a-m

The synthesis of SML16 was shown in Scheme 16. Representative procedures were described below.

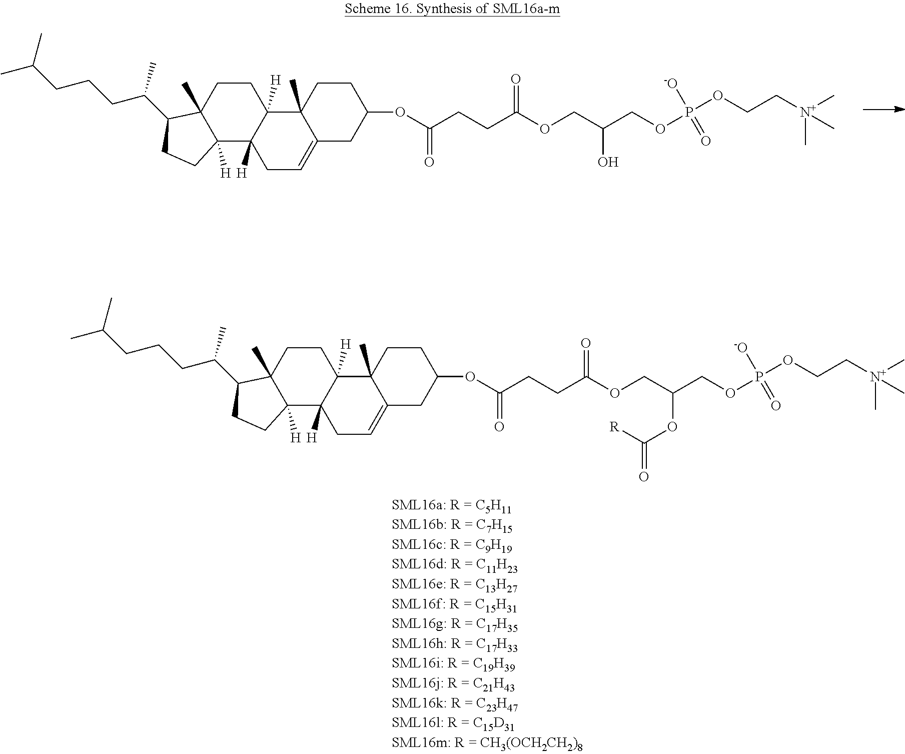

1-cholesterylhemisuccinoyl-2-hydroxyl-3-glycero-phosphocholine was a side product in the synthesis of SML6B (See example 6) and was used as the starting material in the synthesis of SML16a-m.

1-cholesterylhemisuccinoyl-2-palmitoyl-sn-glycero-3-phosphocholine (SML16f, ChemsPPC): To a To a solution of 1-cholesterylhemisuccinoyl-2-hydroxyl-3-glycero-phosphocholine (206 mg, 0.28 mmol) and palmitic acid (87.3 m g, 0.34 mmol) in ethanol-free dry chloroform (6 mL) at room temperature, were added DMAP (35 mg, 0.28 mmol) and DCC (70.2 mg, 0.34 mmol). The reaction mixture was stirred at room temperature (r.t.) for 48 h. The mixture was filtered and the filtrate was concentrated by rotary evaporation. The residue was applied to HPFC for purification (CHCl₃ to CHCl₃-MeOH—H₂O 65/25/4). TLC: R_{f}=0.34 (eluent C). ¹H NMR (CDCl₃), δ0.67 (s, 3H); 0.85-1.65 (m, 62H); 1.84-2.05 (m, 5H); 2.20 (t, J=9, 4H); 2.50 (m, 4H); 3.31 (s, 9H); 3.78 (m, 2H); 3.88 (m, 2H); 4.09 (m, 1H); 4.28 (m, 3H); 4.50 (m, 1H); 5.10 (m, 1H); 5.27 (d, J=4.2, 1H). MALDI-MS calcd for C₅₅H₉₉NO₁₀P⁺ [M+H]⁺ 964.71. Found 964.73.

1-cholesterylhemisuccinoyl-2-(2,5,8,11,14,17,20,23-octaoxahexacosan-26-oyl)-sn-glycero-3-phosphocholine (SML16m, ChemsPEO8PC): To a To a solution of 1-cholesterylhemisuccinoyl-2-hydroxyl-3-glycero-phosphocholine (140 mg, 0.193 mmol) and 2,5,8,11,14,17,20,23-octaoxahexacosan-26-oic acid (100 m g, 0.242 mmol) in ethanol-free dry chloroform (6 mL) at room temperature, were added DMAP (20 mg) and DCC (60 mg, 0.29 mmol). The reaction mixture was stirred at r.t. for 48 h. The mixture was filtered and the filtrate was concentrated by rotary evaporation. The residue was applied to HPFC for purification (CHCl₃ to CHCl₃-MeOH—H₂O 65/25/4). TLC: R_{f}=0.47 (eluent C). ¹H NMR (CDCl₃), δ0.67 (s, 3H); 0.85-1.65 (m, 33H); 1.84-2.05 (m, 5H); 2.29-2.31 (m, 2H); 2.55-2.62 (m, 6H); 3.31 (s, 9H); 3.37 (s, 3H); 3.54-3.69 (m, 30H); 3.81 (m, 2H); 3.98 (m, 2H); 4.16 (m, 1H); 4.35 (m, 3H); 4.58 (m, 1H); 5.21 (m, 1H); 5.35 (d, J=4.4, 1H). MALDI-MS calcd for C₅₇H₁₀₃NO₁₈P⁺ [M+H]⁺ 1120.70. Found 1120.64.

### Example 37

### Applications of SML16m

SML16m was designed to have a large surface area than conventional phospholipids by replacing of the hydrophobic lipid chain with an amphiphilic poly(ethylene glycol) chain. SML16m dissolves in water easily. At a concentration of 10 mM, SML16m doesn't form detectable particles in water. Unlike conventional amphiphilic molecules, SML16m solution doesn't foam when agitated, but forms a thin membrane along the glass wall above the solution surface. SML16m can effectively solubilize amphotericin B at molar ratios of 1:1 or higher to obtain a concentration of 5 mg/mL. SML16m can also be formulated with other lipids to achieve small liposomes. For example, the particle diameter of multilamellar vesicles of DPPC was significantly reduced when 20 mol % SML16m was added.

### Example 38

### Controlled Release of 5-Carboxyfluorescein from SML-Containing Liposomes in 30% Fetal Bovine Serum

Drug release rate from liposome is controlled by the rigidity and permeability of the lipid bilayer. The alkyl chain length and the degree of unsaturation play an important role in liposome formulation. The release of 5-carboxyfluorescein (CF) from SML liposomes was evaluated in HEPES buffer saline (10 mM HEPES, 140 mM NaCl, pH 7.4) (HBS) and 30% fetal bovine serum (FBS).

CF was encapsulated into the liposome by the method similar to that described in Example 16. Liposomes were extruded through 200 nm polycarbonate membrane, and the free CF was removed by eluting liposomes loaded on a PD-10 column (GE Healthcare, Piscataway, N.J.) with HBS as the isomotic eluent. An aliquot of liposome sample (10 μL) was diluted in 96-well plate by either HBS or FBS to a total volume of 200 μL that containing 0.02% sodium azide. The plate was then sealed with transparent plastic film and incubated at 37° C. Fluorescence intensities of samples were monitored at different time points and the percentage of CF released from liposome was determined by the following formula.

CF%=(Fₜ−F₀)/(Fₐ−F₀)*100%

wherein
F₀=background fluorescence signal
Fₐ=total fluorescence signal
Fₜ=fluorescence signal at the time of measurement

The SMLs tested included:

1-Hexanoyl-2-cholesterylhemisuccinoyl-sn-glycero-3-phosphocholine (SML15a)

1-Decanoyl-2-cholesterylhemisuccinoyl-sn-glycero-3-phosphocholine (SML15c)

1-Dodecanoyl-2-cholesterylhemisuccinoyl-sn-glycero-3-phosphocholine (SML15d)

1-Tetradecanoyl-2-cholesterylhemisuccinoyl-sn-glycero-3-phosphocholine (SML15e)

1-Hexadecanoyl-2-cholesterylhemisuccinoyl-sn-glycero-3-phosphocholine (SML15f)

1-Octadecanoyl-2-cholesterylhemisuccinoyl-sn-glycero-3-phosphocholine (SML15g)

1-Oleoyl-2-cholesterylhemisuccinoyl-sn-glycero-3-phosphocholine (SML15h)

1-Icosanoyl-2-cholesterylhemisuccinoyl-sn-glycero-3-phosphocholine (SML15i)

1-Docosanoyl-2-cholesterylhemisuccinoyl-sn-glycero-3-phosphocholine (SML15j)

1,2-Dicholesterylhemisuccinoyl-sn-glycero-3-phosphocholine (SML6b)

Four types of liposome formulations were used in the CF release test:

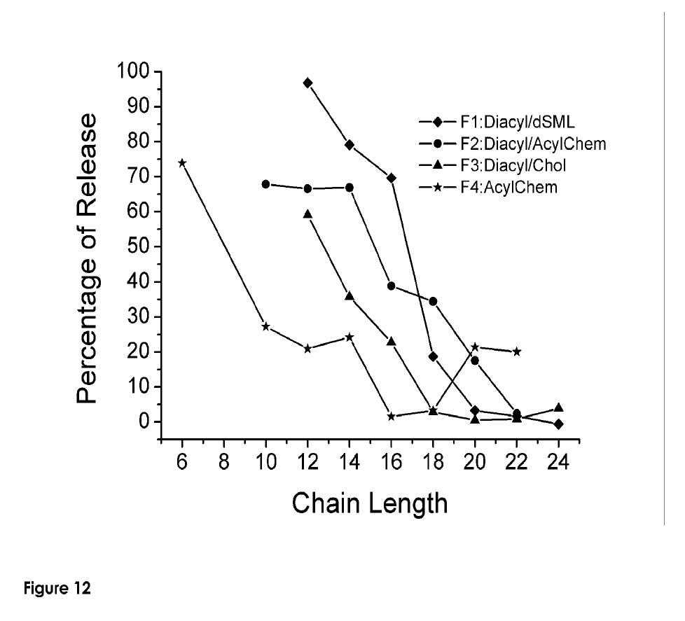

(1) liposomes of SML6b and diacyl phospholipids containing 45 mol % equivalent cholesterol, denoted as F1 in FIG. 12;

(2) liposomes of 1-acyl-2-chems-phosphocholine and diacyl phospholipids of the same chain length, denoted as F2 in FIG. 12;

(3) liposomes of cholesterol and diacyl phospholipid containing 45 mol % cholesterol, denoted as F3 in FIG. 12; and

(4) liposomes of pure 1-acyl-2-chems-phosphocholine (SML15 series), denoted as F4 in FIG. 12.

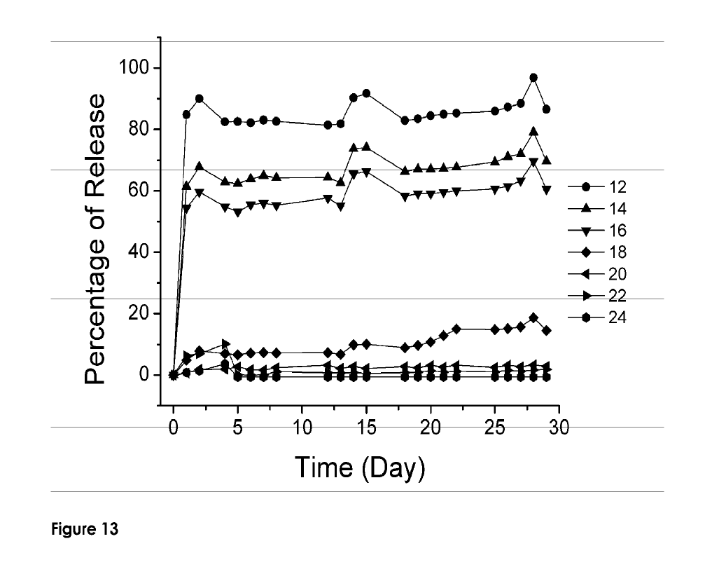

As shown in the results presented in FIG. 12, all formulations containing long chain lipid demonstrated good stability and resistance to leakage. Liposomes of pure 1-acyl-2-chem-PC showed stability in a broad range of chain length started from as low as C10 with less than 30% CF release. However, mixing with diacyl lipid dramatically shifted the curve toward the longer alkyl chain. The diacyl/SML6b liposomes require slightly longer alkyl chains than the conventional diacyl/cholesterol liposomes to reduce the release of CF. It was not possible to prepare a stable liposome from a diacyl/cholesterol mixture where the diacyl chain length is less than 12. This is an additional advantage of the SML formulation F4. The full release profiles of diacyl/SML6b are illustrated in FIG. 13. Together three SML formulations provide a wide range of choices for controlled release of drug at desired rate.

That which is claimed is:

1. A compound comprising a sterol-modified amphiphilic lipid (SML) having a hydrophilic head group and two or more hydrophobic tail groups, wherein at least one of the hydrophobic tail groups comprises a sterol.

2. The compound of claim 1, wherein the head group comprises a phosphate.

3. The compound of claim 2, wherein the hydrophilic head group is selected from the group consisting of phosphate, phosphocholine, phosphoglycerol, phosphoethanolamine, phosphoserine, phosphoinositol, and ethylphosphosphorylcholine.

4. The compound of claim 3, wherein the phosphoethanolamine head group is selected from the group consisting of phosphoethanolamine-N-[monomethoxypolyethyleneglycol] 2000, and phosphoethanolamine-N-succinyl-N-tri-nitriloacetic acid.

5. The compound of claim 2, wherein the sterol is selected from the group consisting of zoosterols and phytosterols.

6. The compound of claim 5, wherein the sterol is selected from the group consisting of cholesterol, steroid hormones, campesterol, sitosterol, ergosterol, and stigmasterol.

7. The compound of claim 2, wherein at least one of the hydrophobic tail groups comprises a non-sterol.

8. The compound of claim 7, wherein the non-sterol hydrophobic tail comprises an aliphatic hydrocarbon that is saturated or unsaturated, linear or branched, substituted or unsubstituted.

9. The compound of claim 2, wherein the SML is selected from the group consisting of a monosterol-modified amphiphilic lipid, and a disterol-modified amphiphilic lipid.

10. The compound of claim 1, wherein the SML is selected from the group consisting of sterol-modified glycerophospholipids, sterol-modified sphingophospholipids, sterol-modified carnitine lipids, and sterol-modified amino acid lipids.

11. The compound of claim 1, wherein the hydrophilic head group is selected from the group consisting of amino acid, activated functional group, melamine, glucosamine, polyamine, carboxylate (COO⁻), sulfate (SO₄⁻), sulfonate (SO₃⁻), branched polyethylene glycol, polyglycerol, tri-nitriloacetic acid, and carbohydrate.

12. The compound of claim 1, wherein one of the hydrophobic tail groups is a prodrug.

13. A sterol-modified amphiphilic lipid selected from the group consisting of: SML1a, SML1b, SML1c, SML2a, SML2b, SML2c, SML2d, SML3a, SML3b, SML3c, SML3d, SML4a, SML4b, SML4c, SML4d, SML5a, SML5b, SML5c, SML5d, SML6a, SML6b, SML6c, SML6d, SML7a, SML7b, SML8a, SML8b, SML8c, SML8d, SML8e, SML8f, SML9a, SML9b, SML9c, SML10a, SML10b, SML10c, SML10d, SML10e, SML10f, SML11a, SML11b, SML11c, SML11d, SML11e, SML11f, SML12a, SML12b, SML12c, SML12d, SML12e, SML12f, SML13a, SML13b, SML13c, SML13d, SML13e, SML13f, SML13g, SML13h, SML13i, SML13j, SML13k, SML14a, SML14b, SML14c, SML14d, SML14e, SML14f, SML15a, SML15b, SML15c, SML15d, SML15e, SML15f, SML15g, SML15h, SML15i, SML15j, SML15k, SML16a, SML16b, SML16c, SML16d, SML16e, SML16f, SML16g, SML16h, SML16i, SML16j, SML16k, SML161, and SML16m, and derivatives thereof.

14. A composition comprising a sterol-modified amphiphilic lipid (SML) according to claim 1.

15. The composition of claim 14, wherein the SML is a sterol-modified amphiphilic phospholipid (SPL).

16. The composition of claim 15, wherein the composition further comprises at least one of a therapeutic agent, a cosmetic agent, and a detectable label.

17. The composition of claim 15, wherein the composition further comprises a non-sterol amphiphilic lipid, wherein the non-sterol amphiphilic lipid comprises an aliphatic hydrocarbon that is saturated or unsaturated, linear or branched, substituted or unsubstituted.

18. The composition of claim 17, wherein the SPL and the non-sterol amphiphilic lipid comprise hydrophobic tail groups that are approximately the same lengths.

19. The composition of claim 18, wherein the SPL is a monosterol-modified amphiphilic phospholipid and the non-sterol amphiphilic lipid is a diacyl phospholipid, each having non-sterol hydrophobic tails with a chain length of about 6 to 24 carbons.

20. A method for synthesis of a sterol-modified amphiphilic lipid, the method comprising:
coupling at least one sterol tail group through a branching core to a hydrophilic head group so as to generate a sterol-modified amphiphilic lipid having a hydrophilic head group linked to two or more hydrophobic tail groups, wherein at least one of the hydrophobic tail groups comprises the sterol tail group.

21. A method for the production of a composition comprising a sterol-modified amphiphilic lipid, the method comprising:
admixing a sterol-modified amphiphilic lipid with at least one of a non-sterol amphiphilic lipid, a therapeutic agent, a cosmetic agent, a detectable label, a buffer, a solvent, and an excipient.

22. A method administering a composition comprising a sterol-modified amphiphilic lipid to an animal or a cell, the method comprising contacting the animal or cell with a composition according to claim 14.

23. A method of detecting the presence or absence of an analyte in fluid, the method comprising:
contacting the fluid with a composition of according to claim 14, and
detecting at least one change in a detectable property of the lipid composition or the fluid.
